(19) United States
(12) Patent Application Publication
(10) Pub. No.: US 2024/0209456 A1
(43) Pub. Date: Jun. 27, 2024

(54) METHODS AND COMPOSITIONS RELATED TO CELL-CYCLE RNA's

(71) Applicants: BETH ISRAEL DEACONESS MEDICAL CENTER, Boston, MA (US); UNIVERSITY OF PORTSMOUTH, Portsmouth (GB)

(72) Inventors: Daniel Geoffrey TENEN, Boston, MA (US); Annalisa DI RUSCIO, Boston, MA (US); Alexander K. EBRALIDZE, Boston, MA (US); Colyn CRANE-ROBINSON, Portmouth (GB); Simone UMMARINO, Boston, MA (US)

(21) Appl. No.: 18/557,277

(22) PCT Filed: Apr. 25, 2022
(86) PCT No.: PCT/US2022/026118

§ 371 Date: Oct. 25, 2023

Related U.S. Application Data
(60) Provisional application No. 63/180,756, filed on Apr. 28, 2021.

(51) Int. Cl. C12Q 1/6886; C12Q 1/6804; C12Q 1/6851; C12Q 1/6869

(52) U.S. Cl. CPC C12Q 1/6886; C12Q 1/6804; C12Q 1/6869; C12Q 1/6851; C12Q 2600/106; C12Q 2600/136; C12Q 2600/158

(57) ABSTRACT

The present disclosure relates to compositions and methods of treating cancer in a subject in need thereof, relating to a specific non-coding RNA (ncRNA)-S Phase Early RNAs (SPEARs) or inhibitors thereof.

### CROSS-REFERENCE TO RELATED APPLICATIONS

This application claims priority to U.S. Provisional Application No. 63/180,756, filed on Apr. 28, 2021, the entire contents of which are incorporated herein.

### GOVERNMENT INTEREST

This invention was made with government support under grant CA197697 and CA211304 awarded by the National Institutes of Health. The government has certain rights in the invention.

### FIELD

The present technology relates to methods and compositions relating to specific non-coding RNA (ncRNA)-S Phase Early RNAs (SPEARs).

### DESCRIPTION OF THE TEXT FILE SUBMITTED ELECTRONICALLY

The instant application contains a Sequence Listing which has been submitted in ASCII format via EFS-Web and is hereby incorporated by reference in its entirety. Said ASCII copy, created about Apr. 18, 2022, is named “BID-011PC_110304-5011_Sequence_Listing_ST25.txt” and is about 19,732 bytes in size.

### BACKGROUND

Functional noncoding RNAs (ncRNAs) are essential components of the chromatin architecture that is shaped by epigenetic marks such as DNA methylation, histone modifications, nucleosome positioning and the incorporation of histone variants into nucleosomes. Despite this importance, many questions about the biology of ncRNAs remain.

Aberrant epigenetic signatures have been linked to deleterious conditions such as cancers and other genetic diseases. However, little is known about active versus inactive epigenetic marks and how they impact deleterious conditions.

What is needed is a more complete understanding of ncRNAs and epigenetic modulation to allow for effective therapies.

### SUMMARY

Therefore, the present disclosure provides, in aspects, a method for treating cancer in a subject in need thereof, comprising administering: (i) an effective amount of an inhibitor of one or more S Phase Early RNAs (SPEARs) to the subject or (ii) an effective amount of a cell derived from the subject, the cell having been contacted with an effective amount of an inhibitor of one or more SPEARs, wherein the cancer is characterized by epigenetic dysregulation.

In aspects, the present disclosure provides a method for preventing an onset or progression of cancer in a subject in need thereof, comprising administering: (i) an effective amount of an inhibitor of one or more SPEARs to the subject or (ii) an effective amount of a cell derived from the subject, the cell having been contacted with an effective amount of an inhibitor of one or more SPEARs, wherein the subject is characterized by a pre-cancerous state comprising epigenetic dysregulation.

In aspects, the present disclosure provides a method for treating a genetic disease or disorder associated with epigenetic dysregulation in a subject in need thereof, comprising administering: (i) an effective amount of an inhibitor of one or more SPEARs to the subject or (ii) an effective amount of a cell derived from the subject, the cell having been contacted with an effective amount of an inhibitor of one or more SPEARs.

In aspects, the present disclosure provides a method for preventing an onset or progression of a genetic disease or disorder associated with epigenetic dysregulation in a subject in need thereof, comprising administering: (i) an effective amount of an inhibitor of one or more SPEARs to the subject or (ii) an effective amount of a cell derived from the subject, the cell having been contacted with an effective amount of an inhibitor of one or more SPEARs.

In aspects, the present disclosure provides a method for resetting the formation of an active histone mark in a cancerous or pre-cancerous cell, comprising administering: (i) an effective amount of one or more SPEARs or an inhibitor of one or more SPEARs to a subject or (ii) contacting a cell derived from the subject with an effective amount of one or more SPEARs or an inhibitor of one or more SPEARs.

In aspects, the present disclosure provides a method for restoring a replication origin complex associated with an undiseased state in a cell characterized by a genetic disease or disorder, comprising administering: (i) an effective amount of one or more SPEARs or an inhibitor of one or more SPEARs to a subject or (ii) contacting a cell derived from the subject with an effective amount of one or more SPEARs or an inhibitor of one or more SPEARs.

In any of the above aspects, the inhibitor causes modulation of levels of expression of one or more genes controlled by the SPEAR. In some embodiments, the modulation of levels of expression of one or more genes controlled by the SPEAR is upregulation of the genes. In some embodiments, the modulation of levels of expression of one or more genes controlled by the SPEAR is downregulation of the genes. In some embodiments, the modulation of levels of expression of one or more genes controlled by the SPEAR is a restoration of levels of the one or more genes as compared to an untreated state. In some embodiments, the one or more genes is an oncogene or proto-oncogene. In some embodiments, the gene is a myc gene. In some embodiments, the myc gene is selected from c-myc (MYC), 1-myc (MYCL), and n-myc (MYCN). In some embodiments, the gene is a tumor suppressor gene.

In some embodiments, one or more SPEARs is overexpressed to generate an artificial replication origin complex. In some embodiments, one or more SPEARs is overexpressed to regulate the direction of replication. In some embodiments, one or more SPEARs is overexpressed and one or more SPEARs' inhibitors (i) slow the progression or prevent the deleterious direction of replication and activates the opposite direction of replication, and/or (ii) modulates the site of a trinucleotide repeat, optionally reducing the size of or reversing the expression of the trinucleotide repeat. In some embodiments, one or more SPEARs is overexpressed and one or more SPEARs' inhibitors treat or prevent a trinucleotide repeat disorder (“TRD”). In some embodiments, the TRD is fragile X syndrome, fragile X-E syndrome, Huntington's disease (HD), spinocerebellar ataxias, a movement disorder, Dentatorubropallidoluysian atrophy, or autism. In some embodiments, the TRD is a polyglutamine (PolyQ) disease and/or a non-polyglutamine disease. In some embodiments, the polyglutamine disease is DRPLA (Dentatorubro-pallidoluysian atrophy), HD (Huntington's disease), SBMA (Spinobulbar muscular atrophy or Kennedy disease), SCA1 (Spinocerebellar ataxia Type 1), SCA2 (Spinocerebellar ataxia Type 2), SCA3 (Spinocerebellar ataxia Type 3 or Machado-Joseph disease), SCA6 (Spinocerebellar ataxia Type 6), SCA7 (Spinocerebellar ataxia Type 7), or SCA17 (Spinocerebellar ataxia Type 17). In some embodiments, the non-polyglutamine disease is FXS (Fragile X syndrome), FXTAS (Fragile X-associated tremor ataxia syndrome), FRAXE (Fragile XE mental retardation), FRDA (Friedreich's ataxia), DM (Myotonic dystrophy), SCA8 (Spinocerebellar ataxia Type 8), SCA12 (Spinocerebellar ataxia Type 12) and premature ovarian failure (POF).

In some embodiments, the one or more SPEARs is overexpressed and one or more SPEARs' inhibitors treat or prevent a TRD by reversing the expansion of a trinucleotide repeat.

In some embodiments, the trinucleotide repeat is selected from CAG, CTG, CGG, and GAA.

In some embodiments, the inhibitor reduces or substantially eliminates epigenetic mark activity associated with the SPEARs.

In some embodiments, the inhibitor reduces or substantially eliminates formation and/or recycling of epigenetic marks.

In some embodiments, the inhibitor reduces or substantially eliminates activation of genes.

In some embodiments, the inhibitor causes the activation of genes.

In some embodiments, the inhibitor reduces or substantially eliminates one or more of DNA methylation, histone modifications, and nucleosome remodeling. In some embodiments, the histone modification is selected from one or more of histone acetylation, phosphorylation, methylation, ubiquitination, and proteolysis, and alterations in chromatin remodeling. In some embodiments, the histone modification is histone acetylation. In some embodiments, the inhibitor causes modulation of disease-causing nucleotide expansions controlled by the SPEAR.

In some embodiments, the inhibitor reduces or substantially eliminates interaction between the SPEAR and one or more histones or histone-associated proteins. In some embodiments, the histone or histone-associated protein is one or more of H1, H2A, H2B, H3, and H4 protein, or a variant thereof. In some embodiments, the histone or histone-associated protein is one or more of H2A.Z, or a variant thereof and H3.3, or a variant thereof. In some embodiments, the histone or histone-associated protein is a histone acetyltransferase. In some embodiments, the histone acetyltransferase is TIP60, or a variant thereof.

In some embodiments, the inhibitor reduces or substantially eliminates interaction between the SPEAR and one or more components of ORC. In some embodiments, the one or more components of ORC is selected from one or more of ORC1, ORC2, ORC3, ORC4, and ORC5, or a variant thereof.

In some embodiments, the epigenetic dysregulation is dysregulation of one or more epigenetic marks.

In some embodiments, the epigenetic dysregulation of one or more epigenetic marks comprises the activation of additional epigenetic marks as compared to undiseased state and/or deactivation of epigenetic marks as compared to undiseased state. In some embodiments, the epigenetic dysregulation is altered replication origin. In some embodiments, the altered replication origin comprises the activation of additional replication origins as compared to undiseased state and/or deactivation of replication origins as compared to undiseased state.

In some embodiments, the subject is afflicted with a cancer associated with epigenetic dysregulation.

In some embodiments, the cancer is a solid tumor. In some embodiments, the cancer is a blood cancer.

In some embodiments, the cancer is one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema, and Meigs' syndrome.

In some embodiments, the SPEAR is a non-coding RNA. In some embodiments, the SPEAR is a long noncoding RNA (lncRNA). In some embodiments, the SPEAR is about 200 nucleotides or longer. In some embodiments, the SPEAR is encoded in a region adjacent to a promoter of an active gene. In some embodiments, the SPEAR is induced in early S phase of the cell cycle. In some embodiments, the SPEAR comprises one or more motifs selected from 3, 5, and 9. In some embodiments, the SPEAR comprises one or more RM9A motifs. In some embodiments, the SPEAR comprises one or more stem-loop-like structures.

In some embodiments, the inhibitor is a small molecule. In some embodiments, the small molecule directly or indirectly modulates interaction of the SPEAR with a histone or histone-associated protein or ORC.

In some embodiments, the inhibitor is a nucleic acid. In some embodiments, the nucleic acid is an RNA or DNA. In some embodiments, the nucleic acid directly or indirectly modulates interaction of the SPEAR with a histone or histone-associated protein or ORC. In some embodiments, the nucleic acid comprising a sequence that is at least partially complementary to a portion of the SPEAR.

In some embodiments, one or more nucleotides of the inhibitor are chemically modified. In some embodiments, the chemical modification is selected from a locked nucleic acid (LNA), phosphorothioate, 2′-O-Methyl, 2′-O-Methoxyethyl, a 2′-O-alkyl-RNA unit, a 2′-OMe-RNA unit, a 2′-amino-DNA unit, a 2′-fluoro-DNA unit, a peptide nucleic acid (PNA) unit, a hexitol nucleic acids (HNA) unit, an INA unit, and a 2′-O-(2-Methoxyethyl)-RNA (2′ MOE RNA) unit.

In some embodiments, the nucleic acid is an antisense oligonucleotide, or a small interfering RNA (siRNA).

In some embodiments, the inhibitor modulates the expression and/or activity of the SPEAR.

In some embodiments, the cell derived from the subject is derived from a biological sample. In some embodiments, the biological sample comprises a biopsy, tissue or bodily fluid. In some embodiments, the biological sample comprises one or more of tumor cells, cultured cells, stem cells, and differentiated cells.

In some embodiments, the methods disclosed herein further comprise administering or contacting the cell with one or more epigenetic drugs. In some embodiments, the epigenetic drug is a DNA methyltransferase inhibitor, optionally selected from azacytidine, ecitabine, zebularine, panobinostat, belinostat, dacinostat, quisinostat, tefinostat, acedinaline, entinostat, mocetinostat, chidamide, butyric acid, pivanex, phenylbutyric acid, and valproic acid. In some embodiments, the epigenetic drug is a histone deacetylase inhibitor, optionally selected from vorinostat, romidepsin, trichostatin A and trapoxin A.

In aspects, the present disclosure provides a method of making an epigenetic modulating agent, comprising: (a) identifying an epigenetic modulating agent by: (i) determining whether the agent binds to or interacts with one or more SPEARs; (ii) classifying the agent as epigenetic modulating based on an ability to bind to or interact with one or more SPEARs; and (b) formulating the agent for use in therapy, the therapy being selected from treatment or prevention of a cancer associated with epigenetic dysregulation or a genetic disease or disorder associated with epigenetic dysregulation.

In some embodiments, the agent reduces or substantially eliminates epigenetic mark activity associated with the SPEARs. In some embodiments, the agent reduces or substantially eliminates formation and/or recycling of epigenetic marks. In some embodiments, the agent reduces or substantially eliminates activation of genes. In some embodiments, the agent causes the activation of genes. In some embodiments, the agent reduces or substantially eliminates one or more of DNA methylation, histone modifications, and nucleosome remodeling. In some embodiments, the histone modification is selected from one or more of histone acetylation, phosphorylation, methylation, ubiquitination, and proteolysis, and alterations in chromatin remodeling. In some embodiments, the histone modification is histone acetylation. In some embodiments, the agent causes modulation of disease-causing nucleotide expansions controlled by the SPEAR. In some embodiments, the agent reduces or substantially eliminates interaction between the SPEAR and one or more histones or histone-associated proteins. In some embodiments, the histone or histone-associated protein is one or more of H1, H2A, H2B, H3, and H4 protein, or a variant thereof. In some embodiments, the histone or histone-associated protein is one or more of H2A.Z, or a variant thereof and H3.3, or a variant thereof. In some embodiments, the histone or histone-associated protein is a histone acetyltransferase. In some embodiments, the histone acetyltransferase is TIP60, or a variant thereof. In some embodiments, the epigenetic dysregulation is dysregulation of one or more epigenetic marks. In some embodiments, the epigenetic dysregulation of one or more epigenetic marks comprises the activation of additional epigenetic marks as compared to undiseased state and/or deactivation of epigenetic marks as compared to undiseased state.

In some embodiments, the epigenetic dysregulation is altered replication origin.

In some embodiments, the SPEAR is a non-coding RNA. In some embodiments, the SPEAR is a long noncoding RNA (lncRNA). In some embodiments, the SPEAR is about 200 nucleotides or longer. In some embodiments, the SPEAR is encoded in a region adjacent to a promoter of an active gene. In some embodiments, the SPEAR is induced in early S phase of the cell cycle. In some embodiments, the SPEAR comprises one or more motifs selected from 3, 5, and 9. In some embodiments, the SPEAR comprises one or more RM9A motifs. In some embodiments, the SPEAR comprises one or more stem-loop-like structures.

In some embodiments, the agent comprises a small molecule. In some embodiments, the small molecule directly or indirectly modulates interaction of the SPEAR with a histone or histone-associated protein or ORC.

In some embodiments, the agent comprises a nucleic acid. In some embodiments, the nucleic acid is an RNA or DNA. In some embodiments, the nucleic acid directly or indirectly modulates interaction of the SPEAR with a histone or histone-associated protein or ORC. In some embodiments, the nucleic acid comprising a sequence that is at least partially complementary to a portion of the SPEAR In some embodiments, one or more nucleotides of the agent is chemically modified.

In some embodiments, the chemical modification is selected from a locked nucleic acid (LNA), phosphorothioate, 2′-O-Methyl, 2′-O-Methoxyethyl, a 2′-O-alkyl-RNA unit, a 2′-OMe-RNA unit, a 2′-amino-DNA unit, a 2′-fluoro-DNA unit, a peptide nucleic acid (PNA) unit, a hexitol nucleic acids (HNA) unit, an INA unit, and a 2′-O-(2-Methoxyethyl)-RNA (2′ MOE RNA) unit.

In some embodiments, the nucleic acid is an antisense oligonucleotide, or a small interfering RNA (siRNA). In some embodiments, the agent is capable of modulating the expression and/or activity of the SPEAR.

In aspects, the present disclosure provides a method for evaluating a subject's response to an epigenetic modulating therapy, comprising evaluating a level of one or more of SPEARs in a biological sample from the subject, wherein: (i) a reduced level of one or more of SPEARs compared to a pretreatment state is indicative of a response to therapy, and/or (ii) an increased or substantially unchanged level of one or more of SPEARs compared to a pretreatment state is indicative of a lack of or poor response to therapy.

In aspects, the present disclosure provides a method for predicting a subject's likelihood of response to an epigenetic modulating therapy, comprising evaluating a level of one or more of SPEARs in a biological sample from the subject, wherein: (i) a high level of one or more of SPEARs is indicative of a high likelihood of response to the therapy, and/or (ii) a low level of one or more of SPEARs is indicative of a low likelihood of response to the therapy.

In some embodiments, the SPEAR is a non-coding RNA. In some embodiments, the SPEAR is a long noncoding RNA (lncRNA). In some embodiments, the SPEAR is about 200 nucleotides or longer. In some embodiments, the SPEAR is encoded in a region adjacent to a promoter of an active gene. In some embodiments, the SPEAR is induced in early S phase of the cell cycle. In some embodiments, the SPEAR comprises one or more motifs selected from 3, 5, and 9. In some embodiments, the SPEAR comprises one or more RM9A motifs. In some embodiments, the SPEAR comprises one or more stem-loop-like structures.

In some embodiments, the biological sample comprises a biopsy, tissue or bodily fluid. In some embodiments, the biological sample comprises one or more of tumor cells, cultured cells, stem cells, and differentiated cells.

The details of one or more examples of the disclosure are set forth in the description below. Other features or advantages of the present disclosure will be apparent from the following drawings, detailed description of several examples, and also from the appended claims. The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### DESCRIPTION OF THE DRAWINGS

FIGS. 1A-1E shows graphs and images characterizing CEBPA and c-MYC SPEARs. FIG. 1A (upper panel) shows a diagram of the CEBPA locus. Lower panel: levels of coding (mRNA), ecCEBPAs (DNMT1-interacting) and UpTr (SPEARs) transcripts immediately after release from double thymidine block. Induction of the CEBPA SPEARs (UpTr) preceded and exceeded expression of ecCEBPA and CEBPA mRNA (qRT-PCR; Bars indicate mean±s.d.). FIG. 1B is a graph showing a genome-wide alignment of the nascent RNAs (nasRNAs). Two-way Venn diagram showing SPEARs specific peaks overlapping with transcribed elements identified in HL-60 total RNA-Seq library. FIG. 1C is a graph showing the correlation between the level of SPEARs and the transcription status of their respective coding genes. Violin plots indicate the distributions of SPEARs expression (HL-60 nasRNA-Seq library) ranked according to global gene expression level (HL-60 total RNA-Seq library) in four distinct groups (Very High: log 2RPKM>8; High: 4<log 2RPKM<8; Medium: 2<log 2RPKM<4, Low: log 2RPKM<2). The statistical differences among the different SPEARs expression groups were computed by the Mann-Whitney-Wilcoxon test (**P≤0.05; **P≤0.01; *** P≤0.001). FIG. 1D is an image showing a snapshot of the c-MYC locus. As an example of another gene locus, see—PU1 in FIG. 8D. FIG. 1E is an image and graph showing the levels of c-MYC SPEARs 10 immediately after release from double thymidine block. Induction of c-MYC SPEARs preceded and surpassed expression of c-MYC mRNA (qRT-PCR; Bars indicate mean±s.d.).

FIGS. 2A-2D shows graphs and images of SPEARs interactions with H2A.Z, acH2A.Z and TIP60. FIG. 2A is a graph showing c-MYC SPEARs immunoprecipitated with anti-H2A.Z, -acH2A.Z and -TIP60 antibodies (qRT-PCR, bars indicate mean+/−s.d.). FIG. 2B is a graph showing RIP-Seq peak intensity histograms for the enrichment of acH2A.Z, H2A.Z and TIP60 on the boundaries of coding regions throughout the genome. The density of aligned RIP-Seq tags is normalized per base pair and is averaged over all the genes in the genome. FIG. 2C is a graph showing SPEARs-producing loci overlap with TIP60/H2A.Z (20%) and TIP60/acH2A.Z (32%) datasets. Venn diagrams indicate overlap of total SPEARs with peaks of their corresponding gene loci significantly enriched by H2A.Z (Upper panel), acH2A.Z (Lower panel) and TIP60. The genes from all four SPEARs expression groups (light yellow circles) intersect with the H2A.Z and acH2A.Z (green circles), TIP60 (blue circles), datasets. Venn diagrams showing overlap of the individual SPEARs expression groups with H2A.Z, acH2A.Z and TIP60 peaks are presented in FIG. 9B and FIG. 9C. FIG. 2D shows scatter plots of the correlation between global gene expression (HL-60, total RNA-Seq library), SPEARs expression (HL-60, nasRNA-Seq library) and acH2A.Z peak intensity at the boundaries of the TSSs of active genes. The intensity and number of acH2A.Z peaks at the boundaries of the TSSs of genes is plotted against the expression of genes within each SPEARs expression group (Very High: log 2RPKM>8; High: 4<log 2RPKM<8; Medium: 2<log 2RPKM<4, Low: log 2RPKM<2).

FIGS. 3A-3F shows graphs and images of the identification of the SPEARs common binding motifs. FIG. 3A shows a schematic flow chart of the SPEARs motif discovery pipeline. FIG. 3B shows the sequences of the common binding motif “9” (RNA oligonucleotide RM9A) the unrelated RNA oligonucleotides UR1, UR2 and UR3 within the c-MYC SPEARs and the corresponding DNA oligonucleotides are indicated. FIG. 3C distribution of the RM9A motif in acH2A.Z and TIP60 RIP-Seq overlapping peak regions. Sequence Logos, significance and percent enrichment are indicated. FIG. 3D, in the left panel, shows c-MYC SPEARs carrying the identified RM9A binding motif form complexes with histone H2A.Z. FIG. 3D, in the right panel, the same but using acetylated peptides from the N terminal sequence of the H2A.Z. FIG. 3E shows c-MYC SPEARs carrying the identified RM9A binding motif form complexes with TIP60 protein but the upstream RNA and DNA motifs do not. Both RNA and DNA probes were used at the same molar concentration, which corresponds to double the counts/minute for the double-stranded DNA oligonucleotides relative to the single-stranded RNA oligonucleotides. FIG. 3F is an image showing c-MYC SPEARs carrying the identified binding motif must fold into stem-loop structures in addition to the primary sequence requirements. The RNA secondary structures were predicted by RNAfold.

FIGS. 4A-4F shows graphs and images of how the global downregulation of SPEARs leads to decreased occupancy of acH2A.Z at the TSSs of the linked genes. FIG. 4A shows a schematic diagram showing synchronization of HL-60 cells by double thymidine block followed by treatment with RNA Polymerase Inhibitors. Upon release from the block, cells were treated with 0.05% DMSO (control); ActD, 0.8 μM; and DRB, 200 μM. Cells were also spiked with EU analog for downstream Click-iT conversion. After 2 hr cells were treated with Ficoll and crosslinked chromatin and RNA collected. FIG. 4B Scatter distribution graph highlighting the correlation between gene expression and acH2A.Z ChIP-Seq intensity obtained from cells treated with the transcription inhibitors DRB (light blue) and ActD (light red), and the DMSO treated control (green). In the upper subplot, the density of the ChIP-Seq tag counts is depicted irrespective of gene expression. In the rightmost subplot, the density of the gene expression profiles is depicted irrespective of the ChIP-Seq tag count. These data imply a causal relationship between acH2A.Z and gene expression, as treatment with transcriptional inhibitors triggers a significant reduction in the acH2A.Z signal and a shift of the density profile towards lower values. FIG. 4C shows a comparison of the enrichment of H2A.Z and acH2A.Z ChIP-Seq signals surrounding gene TSS loci (±2 kb) following treatment with DRB and ActD. The enrichment was calculated as the area under the curve surrounding the TSS for all genes and transformed using a hyperbolic arcsine function. This enabled comparison between the ChIP-Seq occupancy of control (DMSO) samples against the DRB and ActD samples, shown as a scatterplot with a color gradient. In these scatterplots, each dot represents an individual TSS. Regions with a high concentration of TSSs come up in red, while blue represents an absence of TSSs. The differing scales on the plots are indicative of differing antibody characteristics inherent to the H2A.Z and acH2A.Z antibodies. The changes in enrichments are indicated. In FIG. 4D, the upper and middle panels are snapshots of acH2A.Z ChIP-Seq of the c-MYC and PU.1 loci demonstrating strongly diminished acH2A.Z peaks, compared to total H2A.Z, following addition of transcription inhibitors. Full snapshots are shown in FIG. 10D and FIG. 10E. The bottom panel of FIG. 4D shows the MYB locus, which does not show a reduction of the acH2A.Z peaks following addition of transcription inhibitors due to the escape of MYB SPEARs from the action of ActD and DRB. Full snapshots are presented in FIG. 10D and FIG. 10E. FIG. 4E shows the ChIP-qPCR validation of the ChIP-Seq results for the c-MYC locus. Double-headed arrows indicate the position of the qRT-PCR amplicons. (qRT-PCR, bars indicate mean±s.d.). FIG. 4F shows DRB-induced downregulation of the c-MYC and PU.1 SPEARs leads to decreased occupancy of H2A.Z, acH2A.Z and TIP60 at the TSSs of the c-MYC and PU.1 genes. HL-60 cells were released into S Phase and treated with DRB for 2 hrs (as described above). The medium was supplemented with the EdU DNA analog to enable collection of nascent DNAs. Chromatin was collected to perform ChIP assays with antibodies to H2A.Z, acH2A.Z, TIP60 and IgG. Nascent DNAs were isolated from the immunoprecipitated chromatin (see “Methods” below for details and FIG. 8A). Isolated nascent DNAs were analyzed by qPCR (qPCR, bars indicate mean±s.d.).

FIGS. 5A-5C shows graphs of how RNAi-mediated downregulation of the c-MYC SPEARs leads to decreased occupancy of acH2A.Z at the TSSs of the c-MYC gene. FIG. 5A is a graph showing how siRNA-induced downregulation (˜75%) of c-MYCSPEARs lead to ˜70% downregulation of c-MYC mRNA (qRT-PCR, bars indicate mean±s.d.). In FIG. 5B, left panel, snapshots of acH2A.Z ChIP-Seq results at the c-MYC locus are shown, demonstrating diminished acH2A.Z peaks. In FIG. 5B, right panel, the PU.1 locus did not show significant changes in the occupancy of the acH2A.Z (negative control). Full snapshots are shown in FIGS. 10F and 10G. FIG. 5C shows ChIP-qPCR validation of the ChIP-Seq results of RNA interference for the c-MYC locus. Double-headed arrows indicate the position of the qRT-PCR amplicons. qRT-PCR, bars indicate mean+/−s.d.

FIGS. 6A-6D are graphs and images showing how SPEARs regulate the expression of their linked mRNA via a TIP60/acH2AZ pathway. FIG. 6A is a graph showing the response of total c-MYC mRNA and SPEARs to TIP60/HAT inhibitors. c-MYC mRNAs are downregulated by MG149 and TH1834 but c-MYC SPEARs are not affected. HL-60 cells were released from double thymidine block and treated for 2 hrs with MG149 (200 μM) and TH1834 (500 μM); control (mock) treatments were supplemented with DMSO (0.05%) or water, respectively. qRT-PCR and strand-specific qRT-PCR were used to quantitate SPEARs and mRNA. Bars indicate mean±s.d. FIG. 6B is a schematic showing an outline of the experimental design. HL-60 cells were released into S Phase and treated with DMSO/DRB for two hours (as described in FIG. 4A). After 2 hrs, DRB-treated cells were washed and incubated for another 2 hrs with and without TIP60 inhibitors. The medium was supplemented with the EU RNA and EdU DNA analogs to enable collection of nascent RNAs/DNAs. Chromatin was collected to perform ChIP assays with antibodies to H2A.Z, acH2A.Z, TIP60 and IgG. Nascent DNAs and RNAs were isolated from the immunoprecipitated chromatin and total RNA, respectively (see “Methods” below for details, and FIG. 8A). Isolated nascent DNAs and RNAs were analyzed by qPCR and qRT-PCR/strand-specific qRT-PCR, respectively. FIG. 6C shows graphs of different responses of nascent c-MYC SPEARs and mRNA to TIP60/HAT inhibitors: c-MYC mRNAs are downregulated but c-MYC SPEARs are not affected. Quantitation was performed by qRT-PCR (for mRNA) and strand-specific qRT-PCR (for SPEARs). Bars indicate mean±s.d. FIG. 6D shows graphs of nascent ChIP-qPCRs for the c-MYC locus using antibodies to: H2A.Z, acH2A.Z and TIP60 with IgG control. These experiments demonstrate: (i) DRB treatment (which leads to downregulation of c-MYC SPEARs) results in the loss of acH2A.Z (middle panel) and TIP60 (RH panel) and a partial loss of H2A.Z (LH panel); (ii) Restoration of c-MYCSPEARs (after DRB reversal) leads to reappearance of H2A.Z, acH2A.Z and TIP60; and (iii) Inhibition of TIP60 activity by MG149 and TH1834 prevents the restoration of acH2A.Z, while only slightly affecting the restoration of TIP60 and H2A.Z distribution. Quantitation by qPCR. Bars indicate mean±s.d. (n=2).

FIG. 7 is an image showing a non-limiting model for establishment of the activating epigenetic acetylation mark on histone H2A.Z (without wishing to be bound by theory). During the early S phase, SPEARs interact with both histone H2A.Z and the acetyltransferase TIP60. H2A.Z and TIP60 achieve physical proximity, leading to a high local effective protein concentration that favors H2A.Z acetylation and exchange with the canonical H2A form within the nucleosome. In this active chromatin conformation, the RNAPII complex engages the site and gene expression is initiated.

FIGS. 8A-8H shows images and graphs of the identification of the S phase Early RNAs (SPEARs). FIG. 8A is a schematic diagram showing synchronization of HL-60 cells by double thymidine block. Upon release from double thymidine block, cells were spiked with analog EU for downstream Click-iT conversion. After 1 hour, total and nuclei RNA were collected. RNAs were processed according to the manufacturer's recommendation and RNA-seq libraries were generated, sequenced and analyzed. FIG. 8B show an enrichment plot of SPEARs expression in the loci of the genes belonging to four different expression groups (Very High: log 2RPKM>8; High: 4<log 2RPKM<8; Medium: 2<log 2RPKM<4, Low: log 2RPKM<2). Genes were classified into four different expression groups according to HL-60 total RNA-seq library, and the SPEARs expression measured according to HL-60 nasRNA-seq library was used to calculate the read count enrichment in the regions covering the upstream regulatory region, gene body and non-coding downstream region. FIG. 8C shows an enrichment heatmap of SPEARs expression in genes belonging to different expression groups (Very High: log 2RPKM>8; High: 4<log 2RPKM<8; Medium: 2<log 2RPKM<4, Low: log 2RPKM<2). The SPEARs expression is plotted in the upstream regulatory region, gene body, and downstream non-coding region for individual genes throughout the genome, showing the relative positional enrichment of different genomic regions within each gene locus. FIG. 8D shows a snapshot of the PU1 gene locus presenting respective SPEARs. FIG. 8E is a diagram of c-MYC locus transcripts. Vertical arrows indicate TSS and TES, dashed arrow indicate position of primer in Primer extension experiment. FIG. 8F is a graph showing c-MYC SPEARs copy number (Experimental details are available in “Methods” below). FIG. 8G shows primer extension experiments. Shown is the radio autograph of the primer extension reactions for the c-MYC SPEARs; radio autographs for SPEARs corresponding to gene loci CEBPA, CTCF and PU.1 are not shown. Black arrows indicate the longest extension product. Dashed arrows indicate “strong-stops” of the extension reactions (Experimental details are available in “Methods” below). FIG. 8H shows the 5′3′ Rapid Amplification of cDNA Ends (“RACE”). The “longest” isoform of the c-MYC SPEARs: TSS at −2160 nt to c-MYC mRNA TSS; and TES at −38 nt to c-MYC mRNA TSS (nucleotide positions marked by the dark arrows). TSSs and TESs for the “shorter” isoforms are indicated by the dashed arrows (for details, see “Methods” below for details).

FIGS. 9A-9C are images and graphs showing SPEARs-H2A.Z-acH2A.Z-TIP 60 interactions. FIG. 9A is a diagram representing generation of biotinylated SPEARs probes and protocol used to pull-down SPEARs-containing RNA-protein complexes (SPEARs-RNPs). Collected soluble SPEARs-RNPs were separated on the 5% PAGE and submitted for Mass spectrometry analyses (Experimental details are available in “Methods” below). FIG. 9B and FIG. 9C are graphs showing the analyses of H2A.Z/acH2A.Z and TIP60 RIP-Seq. Venn diagrams of the overlapping of the set of genes containing significant H2A.Z/acH2A.Z and TIP60 peaks in the promoter regions with the individual SPEARs expression groups (upper circle in each graph). The stratified datasets of genes in four expression groups (Group A—Very High: log 2RPKM>8; Group B—High: 4<log 2RPKM<8; Group C—Medium: 2<log 2RPKM<4: Group D—Low: log 2RPKM<2) were intersected with the sets of genes with H2A.Z/acH2A.Z marks (lower right circle), TIP60 (lower left circle), or both TIP60 and H2A.Z/acH2A.Z marks. (Experimental details are available in “Methods” below).

FIGS. 10A-10G are images and graphs showing how the downregulation of SPEARs leads to decreased occupancy of the acH2A.Z at the TSSs of the respective genes. FIG. 10A is a schematic diagram of the pilot experiment showing synchronization of HL-60 cells by double thymidine block followed by treatment with RNA Polymerase Inhibitors. Upon release from double thymidine block, cells were treated with DMSO; Actinomycin D (ActD; RNA Polymerase I, II and III Inhibitor), 0.8 μM; and 5,6-Dichlorobenzimidazole 1-β-D-ribofuranoside inhibitor (DRB; RNAPII Inhibitor), 200 μM. Cells were also spiked with analogs EU and/or EdU for downstream Click-iT conversion. Cells were collected at different time points and total proteins were subjected to Western blot analyses. Shown are the blot with proteins isolated after 2 hours treatment. These experiments revealed that overall global content of H2A.Z and TIP60 were not affected by drug treatment. FIG. 10B shows a genome wide transcription inhibition upon DRB and ActD treatment. The figure shows the violin plots for the distributions of gene expression in the control (DMSO), and the cells treated with transcription inhibitors DRB and ActD. The comparison of the gene expression distributions show significant differences for the DRB treatment versus the control, and highly significant differences for the ActD treatment versus the control in a Mann-Whitney-Wilcoxon test (p<0.05). The gene expression reduction ratio across distributions is DRB/DMSO=0.45, and ActD/DMSO=0.21. For the DRB and ActD treatments the average gene expression reductions were 55% and 79%, respectively. FIG. 10C shows individual SPEARs inhibition upon DRB and ActD treatment qRT-PCR quantitations of the effects of the DRB and ActD on c-MYC, PU.1 and MYB SPEARs. FIG. 10D and FIG. 10E show how the global downregulation of SPEARs leads to the decreased occupancy of the acH2A.Z at the TSSs of the respective genes. FIG. 10D shows full snapshots of H2A.Z ChIP-Seq of unmodified H2A.Z peaks upon DRB and ActD treatments. FIG. 10E shows full snapshots of acH2A.Z ChIP-Seq of the gene loci demonstrating diminishing of the acH2A.Z peaks (genes: c-MYC and PU.1) and gene locus with unchanged acH2A.Z peaks (gene: MYB) upon DRB and ActD treatments. FIG. 10F and FIG. 10G show RNAi-mediated downregulation of c-MYC SPEARs lead to the decreased occupancy of the acH2A.Z at the c-MYC gene TSS and not H2A.Z. FIG. 10F shows full snapshots of H2A.Z ChIP-Seq of the targeted (c-MYC) and non-targeted (PU.1) gene loci demonstrating no changes of H2A.Z peaks after siRNA-induced downregulation of the c-MYC SPEARs. FIG. 10G shows full snapshots of acH2A.Z ChIP-Seq of the targeted (c-MYC) gene locus showing diminishing of the acH2A.Z peaks after siRNA-induced downregulation of the c-MYC SPEARs, while no effect is observed in non-targeted gene locus (PU.1).

FIGS. 11A-11E are images and graphs showing how SPEARs regulate the expression of the respective mRNA via TIP60/acH2AZ recruitment/deposition. FIG. 11A is a graph of a blot with proteins isolated after 2 hours treatment. Upon release from double thymidine block, cells were treated with DMSO; MG149 either 100 or 200 μM; and TH1834, 250 or 500 μM. Cells were collected at different time points and total proteins were subjected to Western blot analyses. Reduction of acH2A.Z was observed with 200 μM of MG149 and 500 μM of TH1834. Quantification of the bands was obtained using ImageJ; levels of acH2A.Z were normalized by the levels of H2A.Z. FIG. 11B shows how the downregulation of c-MYC and c-MYC SPEARs (after DRB treatment) leads to the loss of acH2A.Z and TIP60 enrichment, “DRB” bars (middle and right panels) and restoration of c-MYC and c-MYC SPEARs (after DRB reversal). qRT-PCR and strand-specific qRT-PCR; bars indicate mean+/−s.d. FIG. 11C are graphs showing nascent ChIP-qPCRs for a GENE DESERT locus showing no changes in levels of enrichment for H2A.Z, acH2A.Z and TIP60. qPCR, bars indicate mean+/−s.d. (n=2). FIG. 11D are graphs showing different responses of total PU1 SPEARs and mRNA to TIP60/HAT inhibitors: PU1 mRNAs are downregulated while PU1 SPEARs are not affected. qRT-PCR and strand-specific qRT-PCR; bars indicate mean+/−s.d. FIG. 11E shows nascent ChIP-qPCRs for the PU1 locus demonstrating: (i) Downregulation of PU1 SPEARs (after DRB treatment) leads to the loss of acH2A.Z and TIP60 enrichment, “DRB” bars (middle and right panels); (ii) Restoration of PU1 SPEARs (after DRB reversal) leads to re-occurrence of the acH2A.Z and TIP60 enrichment, “DRB REV” bars (middle and right panels); (iii) Inhibition of TIP60/HAT activity (after MG149 and TH1834 treatments) is preventing the restoration of the acH2A.Z enrichment without affecting the restoration of TIP60 enrichment, TH1834 and MG149 bars (middle and right panels); qPCR, bars indicate mean+/−s.d. (n=2).

FIG. 12A, FIG. 12B, FIG. 12C, and FIG. 12D are images showing how the induction of SPEARs-like transcription affects the size of trinucleotide repeats. In FIG. 12A (without wishing to be bound by theory), and as a non-limiting example, sample #1 refers to a model of MyoD-generated “myocytes” from fibroblasts isolated from Myotonic dystrophy type 1 (DM1) subjects, which, without wishing to be bound by theory, leads to induction of bi-directional transcription within the DMPK gene locus. Sample #2 refers to non-treated fibroblasts isolated from DM1 subjects. FIG. 12B and FIG. 12C show how genomic DNAs were extracted from Samples #1 and #2 and underwent PCR. A primer pair flanks the CTGn repeat area. FIG. 12C shows an interpretation of the PCR bands. Sample #2 shows how the wild-type DMPK allele generates a band of 150 nucleotides (nt) (black dot/black arrow), and a mutant DMPK allele generates a band of 450 nt (red dot/red arrow). For, sample #1, similar bands are present as seen for sample #2, however an additional band at 450 nt is present, as the mutant DMPK allele generates bands of ˜800 nt (expanded mutant allele; blue arrow) and of ˜350 nt (contracted mutant allele; purple arrow). FIG. 12D shows sanger-sequencing of the CTGn-carrying PCR bands shown in FIG. 12C.

### DETAILED DESCRIPTION

Prior to the present disclosure, mechanisms by which epigenetic modifications are established in gene regulatory regions of active genes has remained elusive. The present disclosure demonstrates, inter alia, that the establishment of a major epigenetic mark, the acetylated form of the replacement histone H2A.Z, is regulated by cell cycle-specific long noncoding RNAs encoded in regions adjacent to the promoters of active genes. These transcripts, termed SPEARs (S Phase EArly RNAs), are induced in early S phase: their expression precedes that of the downstream genes on which they exert their regulatory action. SPEARs set the stage for the modification and deposition of the acetylated form of histone H2A.Z by bringing together the replacement histone and the histone acetyl transferase TIP60. As disclosed herein, this widespread bimodal interaction constitutes a novel RNA-mediated mechanism for the establishment of epigenetic marks and cell-specific epigenetic profiles, providing a unifying mechanistic explanation for the accuracy and persistence of epigenetic marks on chromatin.

The present disclosure is based, in part, on the discovery of compositions and methods related to treating cancer in a subject in need thereof, comprising administering: (i) an effective amount of an inhibitor of one or more S Phase Early RNAs (SPEARs) to the subject or (ii) an effective amount of a cell derived from the subject, the cell having been contacted with an effective amount of an inhibitor of one or more SPEARs. In some embodiments, the cancer is characterized by epigenetic dysregulation.

In various embodiments, disclosed herein is a method for preventing an onset or progression of cancer in a subject in need thereof, comprising administering: (i) an effective amount of an inhibitor of one or more SPEARs to the subject or (ii) an effective amount of a cell derived from the subject, the cell having been contacted with an effective amount of an inhibitor of one or more SPEARs, wherein the subject is characterized by a pre-cancerous state comprising epigenetic dysregulation.

In various embodiments, disclosed herein is a method for treating a genetic disease or disorder associated with epigenetic dysregulation in a subject in need thereof, comprising administering: (i) an effective amount of an inhibitor of one or more SPEARs to the subject or (ii) an effective amount of a cell derived from the subject, the cell having been contacted with an effective amount of an inhibitor of one or more SPEARs.

In various embodiments, disclosed herein is a method for preventing an onset or progression of a genetic disease or disorder associated with epigenetic dysregulation in a subject in need thereof, comprising administering: (i) an effective amount of an inhibitor of one or more SPEARs to the subject or (ii) an effective amount of a cell derived from the subject, the cell having been contacted with an effective amount of an inhibitor of one or more SPEARs.

In various embodiments, disclosed herein is a method for resetting the formation of an active histone mark in a cancerous or pre-cancerous cell, comprising administering: (i) an effective amount of one or more SPEARs or an inhibitor of one or more SPEARs to a subject or (ii) contacting a cell derived from the subject with an effective amount of one or more SPEARs or an inhibitor of one or more SPEARs.

In various embodiments, disclosed herein is a method for restoring a replication origin complex associated with an undiseased state in a cell characterized by a genetic disease or disorder, comprising administering: (i) an effective amount of one or more SPEARs or an inhibitor of one or more SPEARs to a subject or (ii) contacting a cell derived from the subject with an effective amount of one or more SPEARs or an inhibitor of one or more SPEARs. For example, administering an effective amount of one or more SPEARs, or an inhibitor of one or more SPEARs, to a subject restores a replication origin complex associated with an undiseased state in a cell characterized by a genetic disease or disorder, by restoring the expression levels of one or more SPEARs, restoring the replication competence of the replication origin complex, and/or by the reappearance of a histone or a histone-associated protein (e.g., a histone acetyltransferase, H2A.Z, H3.3, or variants thereof). In some embodiments, one or more SPEARs is overexpressed to restore a replication origin complex. In some embodiments, the method comprises contacting a cell derived from the subject with an effective amount of one or more SPEARs or an inhibitor of one or more SPEARs and the replication origin complex is restored by administering an effective amount of one or more SPEARs, and/or by the reappearance of a histone or a histone-associated protein (e.g., a histone acetyltransferase, H2A.Z, H3.3, or variants thereof). In some embodiments, one or more SPEARs is overexpressed to restore a replication origin complex, or to generate an artificial replication origin complex. In some embodiments, one or more SPEARs is overexpressed to regulate the direction of replication. In some embodiments, one or more SPEARs is overexpressed and one or more SPEARs' inhibitors (i) slow the progression or prevent the deleterious direction of replication and activates the opposite direction of replication, and/or (ii) modulates the site of a trinucleotide repeat, optionally reducing the size of or reversing the expression of the trinucleotide repeat. In some embodiments, one or more SPEARs is overexpressed and one or more SPEARs' inhibitors treat or prevent a Trinucleotide repeat disorders (“TRDs”; e.g., Huntington's disease (HD), spinocerebellar ataxias, a movement disorder, autism) by reversing the expansion of trinucleotide repeats (“TNRs”, including CAG, CTG, CGG, and GAA) that occurs during replication and repair. In some embodiments, ChIP assays with antibodies to a histone or a histone-associated protein, as well as PCR and qRT-PCR, detect the restoration of the replication origin complex associated with an undiseased state in a cell characterized by a genetic disease or disorder (e.g., by measuring and quantitating the expression levels of one or more SPEARs, and/or by the reappearance of a histone or a histone-associated protein). In some embodiments, qRT-PCR and strand-specific qRT-PCR assays detect the restoration of the replication origin complex associated with an undiseased state in a cell characterized by a genetic disease or disorder (e.g., by measuring and quantitating the expression levels of one or more SPEARs, and/or by the reappearance of a histone or a histone-associated protein).

In some embodiments, during the early S phase, formation of a major “active” epigenetic mark is driven by the action cell cycle-specific non-coding RNAs (“ncRNAs”) termed “SPEARs”, which are encoded adjacent to the promoters of actively transcribed genes. In some embodiments, locally induced SPEARs bind to the replacement histone H2A.Z and to a nuclear factor, the histone acetyl transferase TIP60, leading to deposition/acetylation of the replacement histone H2A.Z. In this active chromatin conformation, the RNAPII complex engages the site and gene expression is initiated.

In various embodiments, sequencings assays identify SPEARs encoded adjacent to the promoters of actively transcribed genes. In some embodiments, to identify SPEARs, nascent RNA are captured and sequenced in a high-throughput sequencing assay (e.g., nasRNA-seq) to identify transcripts by e.g., mapping RNA-seq reads onto a genome, or assembling reads de novo into contigs, followed by mapping the contigs onto a transcriptome. In nasRNA-seq, cells are first synchronized and labeled for one hour upon release into S phase. Collected RNAs are then biotinylated by click chemistry, followed by isolation on streptavidin beads, and deep-sequencing to produce a nasRNAs library. SPEARs are identified by correlating gene expression levels with transcripts close to transcription start sites (TSS) of coding genes.

In some embodiments, epigenetic dysregulation is a change, or an alteration, in the epigenetic regulation of gene expression. In some embodiments, the epigenetic dysregulation is a change or alteration in an epigenetic mark (e.g., histone modifications, such as histone acetylation, histone methylation) to the DNA of a cell. In some embodiments, epigenetic dysregulation results in the expression or silencing of genes. In some embodiments, an epigenetic mark is a histone modification selected from one or more of histone acetylation, phosphorylation, methylation, ubiquitination, and proteolysis, and alterations in chromatin remodeling. In some embodiments, the histone modification is histone acetylation.

Two elements of gene expression typically include DNA methylation and chromatin modifications. While DNA methylation is a reversible process that down-regulates gene activity by the addition of a methyl group to the five-carbon of a cytosine base, chromatin modifications are carried out by several mechanisms leading to either the upregulation or down-regulation of the associated gene. In some embodiments, to assess changes in DNA methylation, bisulfite modification or bisulfite sequencing (e.g. by DNA sequencing, single nucleotide primer extension, and/or use of methylation-sensitive primers (MSPs)) is disclosed herein to assay changes in DNA methylation. In some embodiments, to assay for epigenetic dysregulation in a cancer, a chromatin immunoprecipitation (ChIP) assay is used to assess for epigenetic changes, as well as the effects of epigenetic modifications of histones (e.g., native ChIP (nChIP), real-time PCR (Q-ChIP), DNA methylation ChIP (ChIP-MSP). In some embodiments, the ChIP assay is ChIP-sequencing (ChIP-seq), which combines ChIP with DNA sequencing to identify epigenetic marks, which include DNA-binding proteins, histone modifications or nucleosomes. In some embodiments, the ChIP assay is combined with nasChIP, and/or RNAi, to identify epigenetic marks and gene expression levels. In some embodiments, epigenetic dysregulation is assessed using DNaseI hypersensitivity methods. DNaseI hypersensitivity sites are typically located in or around promoter regions, thereby allowing for mapping of transcriptionally active versus inactive chromatin. In various embodiments, DNA methylation and chromatin modifications are assessed using an assay as described in, for example, DeAngelis, J. et al., Mol Biotechnol. 2008 February; 38(2): 179-183, or Gul, S. Clin Epigenet 9, 41 (2017), the entire contents of which are hereby incorporated by reference.

As disclosed herein, histones are altered in cancer. For example, cancer cells commonly show loss of lysine 16 acetylation and lysine 20 methylation. In some embodiments, epigenetic dysregulation is the local changes at a locus, or the global changes of a genome, in histone acetylation and methylation from ChIP assays, in cancerous or pre-cancerous cells compared to normal or healthy cells. In some embodiments, the epigenetic dysregulation characterizes the cancer. In some embodiments, changes in histone acetylation and methylation from ChIP assays (e.g., ChIP-seq) are used to predict the outcome for treating a cancer in a subject, or to predict a subject's likelihood of response to an epigenetic modulating therapy.

In some embodiments, ChIP assays are disclosed herein to assess epigenetic dysregulation in a genomic sequence from a cancer. The ChIP assay typically refers to the process comprising the (1) isolation of chromatin to be analyzed from cells; (2) immunoprecipitation of the chromatin using an antibody; and (3) DNA analysis. In a ChIP assay, fragments of the DNA-protein complex that package the DNA in living cells (i.e., the chromatin), is prepared to retain the specific DNA-protein interactions that characterize each living cell. These chromatin (i.e., the protein-DNA complex) fragments can then be immunoprecipitated using an antibody against the protein in question. The isolated chromatin fraction can then be treated to separate the DNA and protein components, and the identity of the DNA fragments isolated in connection with a particular protein (i.e., the protein against which the antibody used for immunoprecipitation was directed), can then be determined by Polymerase Chain Reaction (PCR) or other technologies used for identification of DNA fragments of defined sequence.

In some embodiments, the ChIP assay disclosed herein is used to assay epigenetic modifications of any sort, on any gene, or region of the genome of any cell type of interest. Examples of epigenetic marks, which may be caused by modification of DNA in the sample include histone protein modification, non-histone protein modification, and DNA methylation. In some embodiments, an epigenetic mark is a histone modification selected from one or more of histone acetylation, phosphorylation, methylation, ubiquitination, and proteolysis, and alterations in chromatin remodeling. In some embodiments, the histone modification is histone acetylation.

Accordingly, for example, the antibody used in the immunoprecipitation step may be immunospecific for non-histone proteins such as transcription factors, or other DNA-binding proteins. For example, the antibody may be immunospecific for any of the histones H1, H2A, H2B, H3 and H4 and their various post-translationally modified isoforms and variants (e.g., H2A.Z). In some embodiments, the histone or histone-associated protein is one or more of H1, H2A, H2B, H3, and H4 protein, or a variant thereof. In some embodiments, the histone or histone-associated protein is one or more of H2A.Z, or a variant thereof and H3.3, or a variant thereof. In some embodiments, the antibody may be immunospecific for enzymes involved in modification of chromatin, such as histone acetylases or deacetylases, or DNA methyltransferases. In some embodiments, the histone or histone-associated protein is a histone acetyltransferase. In some embodiments, the histone acetyltransferase is TIP60, or a variant thereof. Furthermore, histones may be post-translationally modified in vivo, by defined enzymes, for example, by acetylation, methylation, phosphorylation, ADP-ribosylation, sumoylation and ubiquitination. Accordingly, the antibody may be immunospecific for any of these post-translational modifications.

Following the immunoprecipitation step, the method generally comprises a step of purifying DNA from the isolated protein/DNA fraction. This may be achieved, for example, by the standard technique of phenol-chloroform extraction or by any other purification method known to one of skill in the art.

Following the purification step, the DNA fragments isolated in connection with the protein is analyzed by PCR. For example, the analysis step may comprise use of suitable primers, which during PCR, will result in the amplification of a length of nucleic acid. The skilled artisan will appreciate that the method according to the invention may be applied to analyze epigenetic modifications on any gene or any region of the genome for which specific PCR primers are prepared. For example, ChIP assays use formaldehyde to crosslink DNA and protein, followed by immunoprecipitation of DNA-protein complexes. Once the crosslinks are reversed, the recovered DNA can then be analyzed to measure the amount of DNA bound to a specific protein, e.g., by PCR, or real-time PCR. In some embodiments, the ChIP assay uses micrococcal nuclease digestion to prepare the chromatin for analysis instead of formaldehyde.

In some embodiments, to assess changes in DNA methylation, bisulfite modification or bisulfite sequencing is disclosed herein to assay changes in DNA methylation.

In some embodiments, epigenetic dysregulation is assessed using DNaseI hypersensitivity methods. DNaseI hypersensitivity sites are typically located in or around promoter regions, thereby allowing for mapping of transcriptionally active versus inactive chromatin.

In some embodiments, epigenetic dysregulation in DNA methylation is assessed by bisulfite modification of DNA. Bisulfite modification converts nonmethylated cytosines to uracils, which are then converted to thymines during DNA amplification by PCR, whereas methylated cytosines are protected from bisulfite modification. In some embodiments, bisulfite sequencing is used to analyze bisulfite-treated DNA, and a comprehensive ‘methylome’ (e.g., a pattern of methylated DNA in the genome) map is generated. As disclosed herein, sequencing analysis of bisulfite-modified DNA reveals the methylation status of specific cytosines.

In some embodiments, the combination of bisulfite modification and a ChIP assay disclosed herein allows for the assessment of methylation status and chromatin structure from one biological sample.

In some embodiments, the methods disclosed herein treat a cancer characterized by epigenetic dysregulation in a subject, and/or treat a genetic disease or disorder associated with epigenetic dysregulation in a subject, by administering (i) an effective amount of an inhibitor of one or more SPEARs to the subject or (ii) an effective amount of a cell derived from the subject, the cell having been contacted with an effective amount of an inhibitor of one or more SPEARs.

In some embodiments, the methods disclosed herein prevent an onset or progression of cancer by administering (i) an effective amount of an inhibitor of one or more SPEARs to the subject or (ii) an effective amount of a cell derived from the subject, the cell having been contacted with an effective amount of an inhibitor of one or more SPEARs, wherein the subject is characterized by a pre-cancerous state comprising epigenetic dysregulation.

As a non-limiting example, the prevention of an onset, the presence, and/or the evaluation of the progression of a cancer in a subject can be assessed according to the Tumor/Nodes/Metastases (TNM) system of classification (International Union Against Cancer, 6th edition, 2002), or the Whitmore-Jewett staging system (American Urological Association). Typically, cancers are staged using a combination of physical examination, blood tests, and medical imaging. If tumor tissue is obtained via biopsy or surgery, examination of the tissue under a microscope can also provide pathologic staging. In some embodiments, the stage or grade of a cancer assists a practitioner in determining the prognosis for the cancer and in selecting the appropriate epigenetic modulating therapy.

In some embodiments, the prevention of an onset, or progression, of cancer is assessed using the overall stage grouping as a non-limiting example: Stage I cancers are localized to one part of the body, typically in a small area; Stage II cancers are locally advanced and have grown into nearby tissues or lymph nodes, as are Stage III cancers. Whether a cancer is designated as Stage II or Stage III can depend on the specific type of cancer. The specific criteria for Stages II and III can differ according to diagnosis. Stage IV cancers have often metastasized, or spread to other organs or throughout the body. The onset or progression of cancer can be assessed using conventional methods available to one of skill in the art, such as a physical exam, blood tests, and imaging scans (e.g., X-rays, MRI, CT scans, ultrasound etc.).

As disclosed herein, administering, or administering a treatment/therapy, refers to a treatment/therapy from which a subject receives a beneficial effect, such as the reduction, decrease, attenuation, diminishment, stabilization, remission, suppression, inhibition or arrest of the development or progression of cancer and/or a genetic disease or disorder, or a symptom thereof.

In some embodiments, the methods disclosed herein prevent an onset or progression of cancer characterized by epigenetic dysregulation in a subject in need thereof, and/or an onset or progression of a genetic disease or disorder associated with epigenetic dysregulation.

In some embodiments, the treatment/therapy that a subject receives, or the prevention in the onset of cancer and/or a genetic disease or disorder results in at least one or more of the following effects: (1) the reduction or amelioration of the severity of cancer and/or a genetic disease or disorder, and/or a symptom associated therewith; (2) the reduction in the duration of a symptom associated with cancer and/or a genetic disease or disorder; (3) the prevention in the recurrence of a symptom associated with cancer and/or a genetic disease or disorder; (4) the regression of cancer and/or a genetic disease or disorder, and/or a symptom associated therewith; (5) the reduction in hospitalization of a subject; (6) the reduction in hospitalization length; (7) the increase in the survival of a subject; (8) the inhibition of the progression of cancer and/or a genetic disease or disorder and/or a symptom associated therewith; (9) the enhancement or improvement the therapeutic effect of another therapy; (10) a reduction or elimination in the cancer cell population, and/or a cell population associated with a genetic disease or disorder; (11) a reduction in the growth of a tumor or neoplasm; (12) a decrease in tumor size; (13) a reduction in the formation of a tumor; (14) eradication, removal, or control of primary, regional and/or metastatic cancer; (15) a decrease in the number or size of metastases; (16) a reduction in mortality; (17) an increase in cancer-free survival rate of a subject; (18) an increase in relapse-free survival; (19) an increase in the number of subjects in remission; (20) a decrease in hospitalization rate; (21) the size of the tumor is maintained and does not increase in size or increases the size of the tumor by less 5% or 10% after administration of a therapy as measured by conventional methods available to one of skill in the art, e.g., X-rays, MRI, CAT scan, ultrasound etc; (22) the prevention of the development or onset of cancer and/or a genetic disease or disorder, and/or a symptom associated therewith; (23) an increase in the length of remission for a subject; (24) the reduction in the number of symptoms associated with cancer and/or a genetic disease or disorder; (25) an increase in symptom-free survival of a cancer subject and/or a subject associated with a genetic disease or disorder; and/or (26) limitation of or reduction in metastasis. In some embodiments, the treatment/therapy that a subject receives does not cure cancer, but prevents the progression or worsening of the disease. In certain embodiments, the treatment/therapy that a subject receives does not prevent the onset/development of cancer, but may prevent the onset of cancer symptoms.

In some embodiments, “preventing” an onset or progression of cancer in a subject in need thereof, or “preventing” an onset or progression of a genetic disease or disorder associated with epigenetic dysregulation in a subject in need thereof, is inhibiting or blocking the cancer or genetic disease or disorder. In some embodiments, the methods disclosed herein prevent, or inhibit, the cancer or genetic disease or disorder at any amount or level. In some embodiments, the methods disclosed herein prevent or inhibit the cancer or genetic disease or disorder by at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition, or at least or about a 100% inhibition).

In some embodiments, the subject is characterized by a pre-cancerous state comprising epigenetic dysregulation. In some embodiments, the pre-cancerous state is associated with epigenetic dysregulation of a locus, or with a mutation or a number of mutations. In some embodiments, a pre-cancerous state involves abnormal cells that are at an increased risk of developing into cancer. The pre-cancerous state can be assessed in various ways. For example, screening, such as a physical exam, blood tests, and imaging scans (e.g., X-rays, MRI, CT scans, ultrasound etc.) can check if cancer is present in a subject who is not known previously to have cancer. In some embodiments, characterizing a subject in a pre-cancerous state comprises checking if someone, with suggestive features of cancer (e.g., symptoms or other positive tests), or a “level of pathology” has cancer. A “level of pathology” can refer to level of pathology associated with a pathogen, where the level can be as described above for cancer. When the cancer is associated with a pathogen, a level of cancer can be a type of a level of pathology.

In some embodiments, any gene that is indicative of the development of cancer is used to determine if a subject is characterized by a pre-cancerous state. In embodiments, any of the following genes are used to determine if a subject is characterized by a pre-cancerous state:

| Cancer | Genes |
|---|---|
| Breast cancer (e.g. in | ATM, BARD1, BRCA1, BRCA2, BRIP1 |
| women) | CHEK2, CDH1, NF1, NBN, PALB2, PTEN, |
| | RAD51C, RAD51D, STK11, TP53 |
| Breast cancer (e.g. in | BRCA1, BRCA2, CHEK2, PALB2 |
| men) | |
| Colorectal cancer | EPCAM, MLH1, MSH2, MSH6, PMS2, |
| | CHEK2, PTEN, STK11, TP53, MUTYH |
| Endometrial cancer | EPCAM, MLH1, MSH2, MSH6, PMS2, |
| | PTEN, STK11 |
| Fallopian tube, ovarian, | ATM, BRCA1, BRCA2, BRIP1, EPCAM, |
| primary peritoneal | MLH1, MSH2, MSH6, NBN, PALB2, |
| cancer | RAD51C, RAD51D, STK11 |
| Gastric cancer | CDH1, STK11, EPCAM, MLH1, |
| | MSH2, MSH6, PMS2 |
| Melanoma | BAP1, BRCA2 CDK4, CDKN2A, PTEN, |
| | TP53 |
| Pancreatic cancer | ATM, BRCA1, BRCA2, CDKN2A, EPCAM, |
| | MLH1, MSH2, MSH6, PALB2, STK11, TP53 |
| Prostate cancer | ATM, BRCA1, BRCA2, CHEK2, EPCAM, |
| | MLH1, MSH2, MSH6, PALB2, PMS2 |

In some embodiments, the methods disclosed herein prevent an onset or progression of a genetic disease or disorder associated with epigenetic dysregulation in a subject in need thereof, comprising administering (i) an effective amount of an inhibitor of one or more SPEARs to the subject or (ii) an effective amount of a cell derived from the subject, the cell having been contacted with an effective amount of an inhibitor of one or more SPEARs. While most diseases or disorders have a genetic component, the identification of a genetic disease or disorder typically requires a clinical examination including: 1) a physical examination; 2) an evaluation of medical family history; and/or 3) clinical and laboratory testing. For example, the occurrence of the same condition in more than one family member (e.g., first-degree relatives), multiple miscarriages, stillbirths, and childhood deaths are suggestive of the presence, or onset, or progression of a genetic disease or disorder. In some embodiments, family history of common adult conditions (e.g., heart disease, cancer, dementia) that occurs in two or more relatives at relatively young ages may also suggest a genetic predisposition. In some embodiments, other clinical symptoms that are suggestive of the presence, or onset, or progression of a genetic disease or disorder, which may include developmental delay/mental retardation and congenital abnormalities. Dysmorphologies often involving the heart and facies, as well as growth problems, are suggestive of a genetic disorder caused by an inherited mutation, spontaneous mutation, teratogen exposure, or unknown factors. Some genetic conditions appear during childhood, and others appear in adolescents or adults. Often a genetic disease or disorder can remain undetected for several years until an event such as puberty or pregnancy triggers the onset of symptoms or the accumulation of toxic metabolites that manifests in the disease or disorder.

In some embodiments, the genetic testing comprises cytogenetic, and/or biochemical/molecular testing to detect abnormalities in chromosome structure, protein function, or DNA sequence. Cytogenetics generally involves the examination and staining of whole chromosomes for abnormalities and can reveal distinct bands of each chromosome to show chromosome structure. Biochemical/molecular testing includes detecting whether: (1) a protein is made, (2) too much or too little protein is made, (3) a misfolded protein, (4) an altered active site or other critical region, (5) an incorrectly modified protein, (6) an incorrectly localized protein (buildup of protein), (7) and/or an incorrectly assembled protein.

In some embodiments, the methods disclosed herein reset the formation of an active histone mark in a cancerous or pre-cancerous cell, comprising administering: (i) an effective amount of one or more SPEARs or an inhibitor of one or more SPEARs to a subject or (ii) contacting a cell derived from the subject with an effective amount of one or more SPEARs or an inhibitor of one or more SPEARs. In some embodiments, the reset of formation of an active histone mark is by, for example, histone exchange, e.g., replacement of existing nucleosomes with newly synthesized histones. In some embodiments, histone exchange results in the removal or dilution of preexisting histone modification marks. In some embodiments, a genome-wide ChIP-chip assay is used to identify the reset of formation of an active histone mark in a cancerous or pre-cancerous cell. In some embodiments, and as a non-limiting example, histone exchange delivers histone acetylation epigenetic marks rapidly at a genome-wide scale. In some embodiments, the resetting of the formation of an active histone mark in a cancerous or pre-cancerous cell, takes place by administering: (i) an effective amount of one or more SPEARs or an inhibitor of one or more SPEARs to a subject or (ii) contacting a cell derived from the subject with an effective amount of one or more SPEARs or an inhibitor of one or more SPEARs, which allows for histone exchange and replacement with an active histone mark. For example, administering an effective amount of one or more SPEARs, or an inhibitor of one or more SPEARs, to a subject resets the formation of an active histone mark in a cancerous or pre-cancerous cell by restoring the expression levels of one or more SPEARs, restoring the replication competence of the replication origin complex, and/or by the reappearance of a histone or a histone-associated protein (e.g., a histone acetyltransferase, H2A.Z, H3.3, or variants thereof). In some embodiments, one or more SPEARs is overexpressed to resets the formation of an active histone mark in a cancerous or pre-cancerous cell. In some embodiments, the method comprises contacting a cell derived from the subject with an effective amount of one or more SPEARs or an inhibitor of one or more SPEARs and the formation of an active histone mark in a cancerous or pre-cancerous cell is reset by administering an effective amount of one or more SPEARs, and/or by the reappearance of a histone or a histone-associated protein (e.g., a histone acetyltransferase, H2A.Z, H3.3, or variants thereof). In some embodiments, one or more SPEARs is overexpressed to reset the formation of an active histone mark. In some embodiments, ChIP assays with antibodies to a histone or a histone-associated protein, as well as PCR and qRT-PCR, detect the resetting of the formation of an active histone mark associated in a cancerous or pre-cancerous cell (e.g., by measuring and quantitating the expression levels of one or more SPEARs, and/or by the reappearance of a histone or a histone-associated protein). In some embodiments, qRT-PCR and strand-specific qRT-PCR assays detect the resetting of the formation of an active histone mark associated in a cancerous or pre-cancerous cell (e.g., by measuring and quantitating the expression levels of one or more SPEARs, and/or by the reappearance of a histone or a histone-associated protein).

In some embodiments, one or more SPEARs is overexpressed to restore a replication origin complex, or to generate an artificial replication origin complex. In some embodiments, one or more SPEARs is overexpressed to regulate the direction of replication. In some embodiments, one or more SPEARs is overexpressed and one or more SPEARs' inhibitors (i) slow the progression or prevent the deleterious direction of replication and activates the opposite direction of replication, and/or (ii) modulates the site of a trinucleotide repeat, optionally reducing the size of or reversing the expression of the trinucleotide repeat. In some embodiments, one or more SPEARs is overexpressed and one or more SPEARs' inhibitors treat or prevent a trinucleotide repeat disorders (“TRDs”; e.g., Huntington's disease (HD), spinocerebellar ataxias, a movement disorder, autism) by reversing the expansion of trinucleotide repeats (“TNRs”, including CAG, CTG, CGG, and GAA) that occurs during replication and repair. In some embodiments, the TRD is a polyglutamine (PolyQ) disease and/or a non-polyglutamine disease. In some embodiments, the polyglutamine disease is DRPLA (Dentatorubro-pallidoluysian atrophy), HD (Huntington's disease), SBMA (Spinobulbar muscular atrophy or Kennedy disease), SCA1 (Spinocerebellar ataxia Type 1), SCA2 (Spinocerebellar ataxia Type 2), SCA3 (Spinocerebellar ataxia Type 3 or Machado-Joseph disease), SCA6 (Spinocerebellar ataxia Type 6), SCA7 (Spinocerebellar ataxia Type 7), or SCA17 (Spinocerebellar ataxia Type 17). In some embodiments, the non-polyglutamine disease is FXS (Fragile X syndrome), FXTAS (Fragile X-associated tremor ataxia syndrome), FRAXE (Fragile XE mental retardation), FRDA (Friedreich's ataxia), DM (Myotonic dystrophy), SCA8 (Spinocerebellar ataxia Type 8), SCA12 (Spinocerebellar ataxia Type 12) and premature ovarian failure (POF).

In some embodiments, the methods disclosed herein restore a replication origin complex associated with an undiseased state in a cell characterized by a genetic disease or disorder, comprising administering: (i) an effective amount of one or more SPEARs or an inhibitor of one or more SPEARs to a subject or (ii) contacting a cell derived from the subject with an effective amount of one or more SPEARs or an inhibitor of one or more SPEARs. In some embodiments, the replication origin complex is restored by administering: (i) an effective amount of one or more SPEARs or an inhibitor of one or more SPEARs to a subject or (ii) contacting a cell derived from the subject with an effective amount of one or more SPEARs or an inhibitor of one or more SPEARs.

In some embodiments, the inhibitor causes modulation of levels of expression of one or more genes controlled by the SPEAR. For example, the inhibitor causes modulation of levels of one or more genes selected from RARB2, MSH2, ESR1B, AKR1B1, COL6A2, GPX7, HIST1H3C, HOXB4, RASGRF2, TM6SF1, ARHGEF7, TMEFF2, RASSF1, BRCA1, STRATIFIN, and RASSF1A, which are associated with breast cancer. In some embodiments, the inhibitor causes modulation of levels of one or more genes selected from RUNX3, CDKN2A, and APC, which are associated with gastric, liver, and esophageal cancer, respectively. In some embodiments, the inhibitor causes modulation of levels of one or more genes selected from SEPT9, hMLH1, CDKN2A/p16, HTLF, ALX4, TMEFF2/HPP1, NGFR, SFRP2, NEUROG1, RUNX3, and UBE2Q1, which are associated with colorectal cancer. In some embodiments, the inhibitor causes modulation of levels of one or more genes selected from RARB2, RASSF1A, CHFR, STRATI-FIN, SHOX2, RASSF1A, and APC1, which are associated with lung cancer. In some embodiments, inhibitor causes modulation of levels of one or more genes selected from RARB2, MSH2, ESR1B, AKR1B1, COL6A2, GPX7, HIST1H3C, HOXB4, RASGRF2, TM6SF1, ARHGEF7, TMEFF2, RASSF1, BRCA1, STRATIFIN, RASSF1A, RUNX3, CDKN2A, APC, SEPT9, hMLH1, CDKN2A/p16, HTLF, ALX4, TMEFF2/HPP1, NGFR, SFRP2, NEUROG1, RUNX3, UBE2Q1, RARB2, RASSF1A, CHFR, STRATI-FIN, SHOX2, RASSF1A, and APC1. In some embodiments, the promoter associated with RARB2, MSH2, ESR1B, AKR1B1, COL6A2, GPX7, HIST1H3C, HOXB4, RASGRF2, TM6SF1, ARHGEF7, TMEFF2, RASSF1, BRCA1, STRATIFIN, RASSF1A, RUNX3, CDKN2A, APC, SEPT9, hMLH1, CDKN2A/p16, HTLF, ALX4, TMEFF2/HPP1, NGFR, SFRP2, NEUROG1, RUNX3, UBE2Q1, RARB2, RASSF1A, CHFR, STRATI-FIN, SHOX2, RASSF1A, and APC1 is hypermethylated. In some embodiments, the modulation of levels of expression of one or more genes controlled by the SPEAR is upregulation of the genes. In some embodiments, the upregulation of one or more genes controlled by the SPEAR is selected from RARB2, MSH2, ESR1B, AKR1B1, COL6A2, GPX7, HIST1H3C, HOXB4, RASGRF2, TM6SF1, ARHGEF7, TMEFF2, RASSF1, BRCA1, STRATIFIN, RASSF1A, RUNX3, CDKN2A, APC, SEPT9, hMLH1, CDKN2A/p16, HTLF, ALX4, TMEFF2/HPP1, NGFR, SFRP2, NEUROG1, RUNX3, UBE2Q1, RARB2, RASSF1A, CHFR, STRATI-FIN, SHOX2, RASSF1A, and APC1.

In some embodiments, the modulation of levels of expression of one or more genes controlled by the SPEAR is downregulation of the genes. In some embodiments, the downregulation of expression of one or more genes controlled by the SPEAR is selected from RARB2, MSH2, ESR1B, AKR1B1, COL6A2, GPX7, HIST1H3C, HOXB4, RASGRF2, TM6SF1, ARHGEF7, TMEFF2, RASSF1, BRCA1, STRATIFIN, RASSF1A, RUNX3, CDKN2A, APC, SEPT9, hMLH1, CDKN2A/p16, HTLF, ALX4, TMEFF2/HPP1, NGFR, SFRP2, NEUROG1, RUNX3, UBE2Q1, RARB2, RASSF1A, CHFR, STRATI-FIN, SHOX2, RASSF1A, and APC1.

In some embodiments, the modulation of levels of expression of one or more genes controlled by the SPEAR is a restoration of levels of the one or more genes as compared to an untreated state. In some embodiments, the gene is an oncogene or proto-oncogene. In some embodiments, the oncogene is selected from HER2/neu, RAS, MYC, SRC, BCL2, EGFR, FGFR1, NCOA4, BCL2, FUS, NTRK1, BRCA1, MSH2, WT1, BCL3, GOLGA5, NUP214, BRCA2, NF1, BCL6, GOPC, PAX8, CARS, NF2, BCR, HMGA1, PDGFB, CBFA2T3, NOTCH1, IL2, TNFAIP3, ABL2, EWSR1, MYCL1, ARHGEF12, JAK2, TP53, AKT1, FEV, MYCN, ATM, MAP2K4, and TSC1. In some embodiments, the proto-oncogene is selected from RAS, HER2, MYC, Cyclin D, Cyclin E, BRAF, and BCR-ABL.

In some embodiments, the gene is a myc gene. In some embodiments the myc gene is selected from c-myc (MYC), 1-myc (MYCL), and n-myc (MYCN).

In some embodiments, the gene is a tumor suppressor gene. In some embodiments, the tumor suppressor gene is selected from Rb, p53, VHL, APC, BRCA2, NF1, and/or PTCH.

In some embodiments, the inhibitor reduces or substantially eliminates epigenetic mark activity associated with the SPEARs.

In some embodiments, the inhibitor reduces or substantially eliminates formation and/or recycling of epigenetic marks.

In some embodiments, the inhibitor reduces or substantially eliminates activation of genes.

In some embodiments, the inhibitor causes the activation of genes.

In some embodiments, the inhibitor reduces or substantially eliminates one or more of DNA methylation, histone modifications, and nucleosome remodeling.

In some embodiments, the inhibitor causes modulation of disease-causing nucleotide expansions controlled by the SPEAR.

In some embodiments, the inhibitor reduces or substantially eliminates interaction between the SPEAR and one or more histones or histone-associated proteins.

In some embodiments, the inhibitor reduces or substantially eliminates interaction between the SPEAR and one or more components of ORC. In some embodiments, the one or more components of ORC is selected from one or more of ORC1, ORC2, ORC3, ORC4, and ORC5, or a variant thereof.

In some embodiments, the epigenetic dysregulation is dysregulation of one or more epigenetic marks. In some embodiments, the epigenetic dysregulation of one or more epigenetic marks comprises the activation of additional epigenetic marks as compared to undiseased state and/or deactivation of epigenetic marks as compared to undiseased state. In some embodiments, the epigenetic dysregulation is altered replication origin. In some embodiments, the altered replication origin comprises the activation of additional replication origins as compared to undiseased state and/or deactivation of replication origins as compared to undiseased state.

In some embodiments, the subject is afflicted with a cancer associated with epigenetic dysregulation. In some embodiments, the cancer is a solid tumor. In some embodiments, the cancer is a blood cancer.

In some embodiments, the cancer is one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema, and Meigs' syndrome.

In some embodiments, the SPEAR is a non-coding RNA. In some embodiments, the SPEAR is a long noncoding RNA (lncRNA). In some embodiments, the SPEAR is about 200 nucleotides or longer. In some embodiments, the SPEAR is about 200-10,000 nucleotides in length, or about 200-5000 nucleotides in length, or about 200-1000 nucleotides in length, or about 200-500 nucleotides in length, or about 5000-10000 nucleotides in length, or about 1000-10000 nucleotides in length, or about 500-10000 nucleotides in length. In some embodiments, the SPEAR is about 500 nucleotides in length or longer. In some embodiments, the SPEAR is about 1,000 nucleotides in length or longer. In some embodiments, the SPEAR is about 1,500 nucleotides in length or longer. In some embodiments, the SPEAR is about 2,000 nucleotides in length or longer. In some embodiments, the SPEAR is about 2,500 nucleotides in length or longer. In some embodiments, the SPEAR is about 3,000 nucleotides in length or longer. In some embodiments, the SPEAR is about 3,500 nucleotides in length or longer. In some embodiments, the SPEAR is about 4,000 nucleotides in length or longer. In some embodiments, the SPEAR is about 4,500 nucleotides in length or longer. In some embodiments, the SPEAR is about 5,000 nucleotides in length or longer. In some embodiments, the SPEAR is about 5,500 nucleotides in length or longer. In some embodiments, the SPEAR is about 6,000 nucleotides in length or longer. In some embodiments, the SPEAR is about 6,500 nucleotides in length or longer. In some embodiments, the SPEAR is about 7,000 nucleotides in length or longer. In some embodiments, the SPEAR is about 7,500 nucleotides in length or longer. In some embodiments, the SPEAR is about 8,000 nucleotides in length or longer. In some embodiments, the SPEAR is about 8,500 nucleotides in length or longer. In some embodiments, the SPEAR is about 9,000 nucleotides in length or longer. In some embodiments, the SPEAR is about 9,500 nucleotides in length or longer. In some embodiments, the SPEAR is about 10,000 nucleotides in length.

In some embodiments, the SPEAR is encoded in a region adjacent to a promoter of an active gene. In some embodiments, the SPEAR is induced in the early S phase of the cell cycle. In some embodiments, the SPEAR is induced in the early S phase of the cell cycle and is detected by Flow Cytometry. In various embodiments the detection includes a method described on the world wide web at biotech.illinois.edu/sites/biotech.illinois.edu/files/uploads/cb0804.pdf, the entire contents of which are hereby incorporated by reference.

In some embodiments, during the early S phase, formation of a major “active” epigenetic mark is driven by the action cell cycle-specific non-coding RNAs (“ncRNAs”) disclosed herein as “SPEARs”, which are encoded adjacent to the promoters of actively transcribed genes. In some embodiments, locally induced SPEARs bind to the replacement histone H2A.Z and to a nuclear factor, the histone acetyl transferase TIP60, leading to deposition/acetylation of the replacement histone H2A.Z. In this active chromatin conformation, the RNAPII complex engages the site and gene expression is initiated.

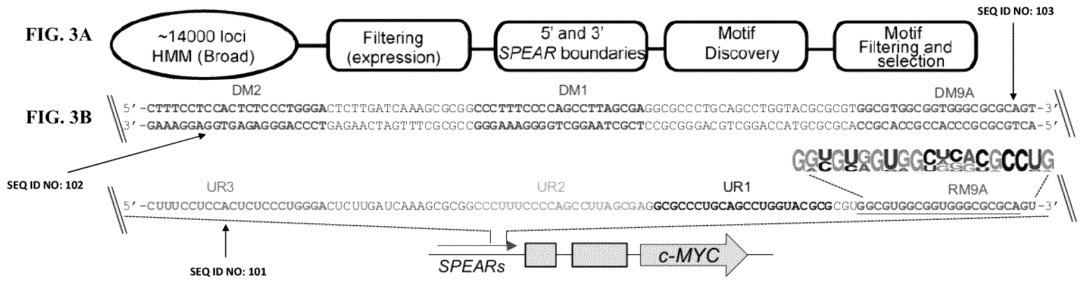

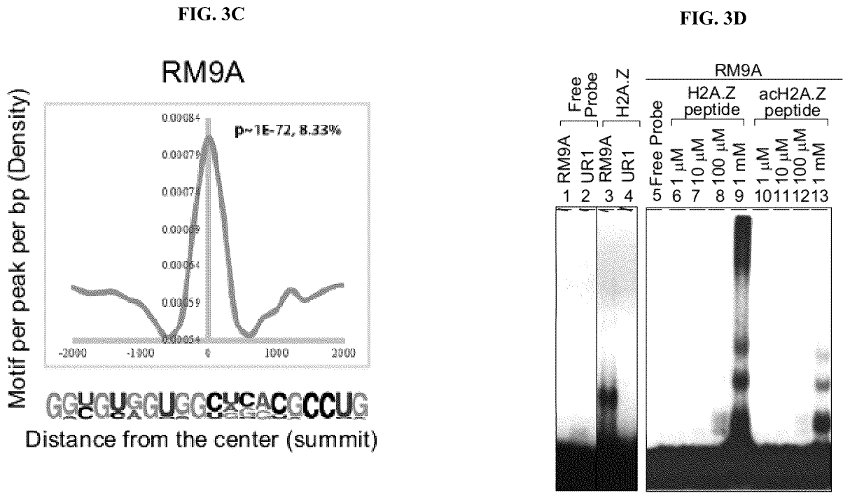

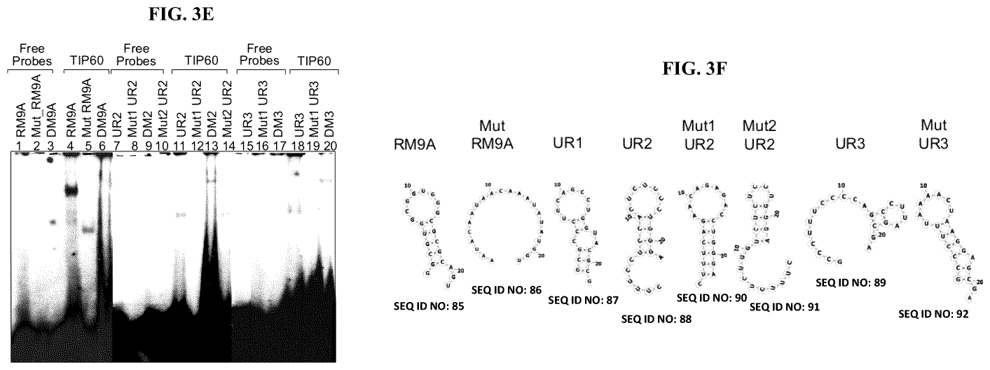

In some embodiments, motif discovery analysis is performed on SPEARs to analyze common binding motifs. In various embodiments, motif discovery analysis is a computational method, as described in Achar, A. et al., Biol Direct 10, 61 (2015), the entire contents of which are hereby incorporated by reference. In some embodiments, promoter loci are subjected to coverage calculation and filtered based on expression level. In some embodiments, the SPEAR comprises one or more motifs selected from 3, 5, and 9. In some embodiments, motif 9 corresponds to RNA oligonucleotide RM9A. In some embodiments, the SPEAR comprises one or more RM9A motifs. In some embodiments, the SPEAR comprises one or more motifs selected from FIG. 3F or a variant thereof.

In some embodiments, SPEAR comprises one or more stem-loop-like structures.

In some embodiments, the inhibitor is a small molecule. In some embodiments, the small molecule directly or indirectly modulates interaction of the SPEAR with a histone or histone-associated protein or ORC. In some embodiments, the inhibitor is a nucleic acid. In some embodiments, the nucleic acid is an RNA or DNA. In some embodiments, the nucleic acid directly or indirectly modulates interaction of the SPEAR with a histone or histone-associated protein or ORC. In some embodiments, the nucleic acid comprising a sequence that is at least partially complementary to a portion of the SPEAR.

In some embodiments, one or more nucleotides of the inhibitor are chemically modified. In some embodiments, the chemical modification is selected from a locked nucleic acid (LNA), phosphorothioate, 2′-O-Methyl, 2′-O-Methoxyethyl, a 2′-O-alkyl-RNA unit, a 2′-OMe-RNA unit, a 2′-amino-DNA unit, a 2′-fluoro-DNA unit, a peptide nucleic acid (PNA) unit, a hexitol nucleic acids (HNA) unit, an INA unit, and a 2′-O-(2-Methoxyethyl)-RNA (2′ MOE RNA) unit.

In some embodiments, the nucleic acid is an antisense oligonucleotide, or a small interfering RNA (siRNA).

In some embodiments, the inhibitor modulates the expression and/or activity of the SPEAR

In some embodiments, the cell derived from the subject is derived from a biological sample.

In some embodiments, the biological sample comprises a biopsy, tissue or bodily fluid.

In some embodiments, the biological sample comprises one or more of tumor cells, cultured cells, stem cells, and differentiated cells.

In some embodiments, the methods further comprise administering or contacting the cell with one or more epigenetic drugs. In some embodiments, the epigenetic drug is a DNA methyltransferase inhibitor, optionally selected from azacytidine, ecitabine, zebularine, panobinostat, belinostat, dacinostat, quisinostat, tefinostat, acedinaline, entinostat, mocetinostat, chidamide, butyric acid, pivanex, phenylbutyric acid, and valproic acid. In some embodiments, the epigenetic drug is a histone deacetylase inhibitor, optionally selected from vorinostat, romidepsin, trichostatin A and trapoxin A.

In some embodiments, disclosed herein is a method of making an epigenetic modulating agent, comprising: (a) identifying an epigenetic modulating agent by: (i) determining whether the agent binds to or interacts with one or more SPEARs; (ii) classifying the agent as epigenetic modulating based on an ability to bind to or interact with one or more SPEARs; and (b) formulating the agent for use in therapy, the therapy being selected from treatment or prevention of a cancer associated with epigenetic dysregulation or a genetic disease or disorder associated with epigenetic dysregulation.

In some embodiments, disclosed herein is a method for evaluating a subject's response to an epigenetic modulating therapy, comprising evaluating a level of one or more of SPEARs in a biological sample from the subject, wherein: (i) a reduced level of one or more of SPEARs compared to a pretreatment state is indicative of a response to therapy, and/or (ii) an increased or substantially unchanged level of one or more of SPEARs compared to a pretreatment state is indicative of a lack of or poor response to therapy. In some embodiments, the epigenetic modulating therapy comprises a drug designed to target an epigenetic mechanism, such as inhibitors of histone deacetylases (HDACs), DNA methyltransferases (DNMTs), enhancer of zeste homologue 2 (EZH2), bromodomain and extra-terminal domain proteins (BETs), protein arginine N-methyltransferases (PRMTs) and isocitrate dehydrogenases (IDHs). In some embodiments, the epigenetic modulating therapy comprises a drug selected from vorinostat, romidepsin, panobinostat belinostat, azacytidine, decitabine, enasidenib, and ivosidenib.

In some embodiments, evaluating a level of one or more of SPEARs in a biological sample from the subject comprises a ChIP assay, sequencing (e.g., ChIP sequencing, RNA sequencing, next generation sequencing (e.g., high-throughput sequencing, deep sequencing), PCR and qRT-PCR In some embodiments, evaluating a level of one or more of SPEARs in a biological sample from the subject comprises capturing nascent RNA, and sequencing the captured RNA in a high-throughput sequencing assay (e.g. nasRNA-seq) to identify the level of transcripts by e.g., mapping RNA-seq reads onto a genome, or assembling reads de novo into contigs, followed by mapping the contigs onto a transcriptome. In nasRNA-seq, cells are first synchronized and labeled for one hour upon release into S phase. Collected RNAs are then biotinylated by click chemistry, followed by isolation on streptavidin beads, and deep-sequencing to produce a nasRNAs library. SPEARs levels are evaluated by correlating gene expression levels with transcripts close to transcription start sites (TSS) of coding genes.

In various embodiments, biological sample refers to a sample obtained or derived from a source of interest (e.g., a cell), as described herein. In certain embodiments, a source of interest comprises an organism, such as an animal or human. In certain embodiments, a biological sample is a biological tissue or fluid. Non-limiting examples of biological samples include bone marrow, blood, blood cells, ascites, (tissue or fine needle) biopsy samples, cell-containing body fluids, free floating nucleic acids, sputum, saliva, urine, cerebrospinal fluid, peritoneal fluid, pleural fluid, feces, lymph, gynecological fluids, swabs (e.g., skin swabs, vaginal swabs, oral swabs, and nasal swabs), washings or lavages such as a ductal lavages or broncheoalveolar lavages, aspirates, scrapings, specimens (e.g., bone marrow specimens, tissue biopsy specimens, and surgical specimens), feces, other body fluids, secretions, and/or excretions, and cells therefrom, etc.

In various embodiments, the “subject” refers to any animal (e.g., a mammal), including, but not limited to, humans, and non-human animals (including, but not limited to, non-human primates, dogs, cats, rodents, horses, cows, pigs, mice, rats, hamsters, rabbits, and the like (e.g., which is to be the recipient of a particular treatment, or from whom cells are harvested)). In some embodiments, the subject is a human.

It will also be understood that, although the terms first, second, etc., may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

This disclosure is further illustrated by the following non-limiting examples.

### EXAMPLES

### Example 1: SPEARs Expression Correlates with that of Neighboring Genes

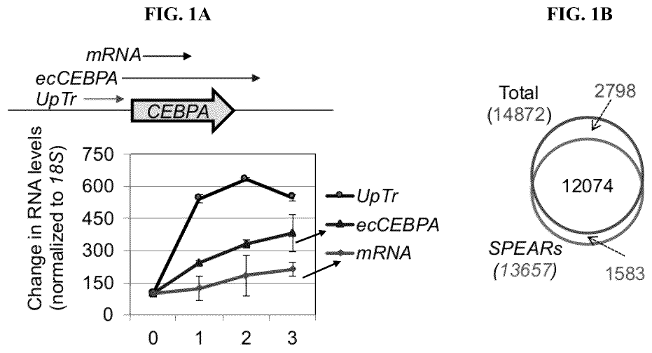

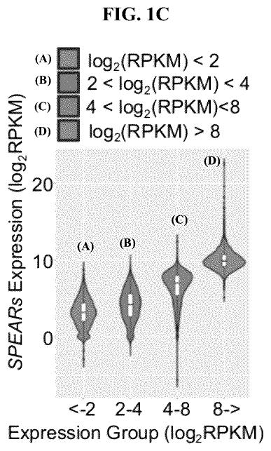

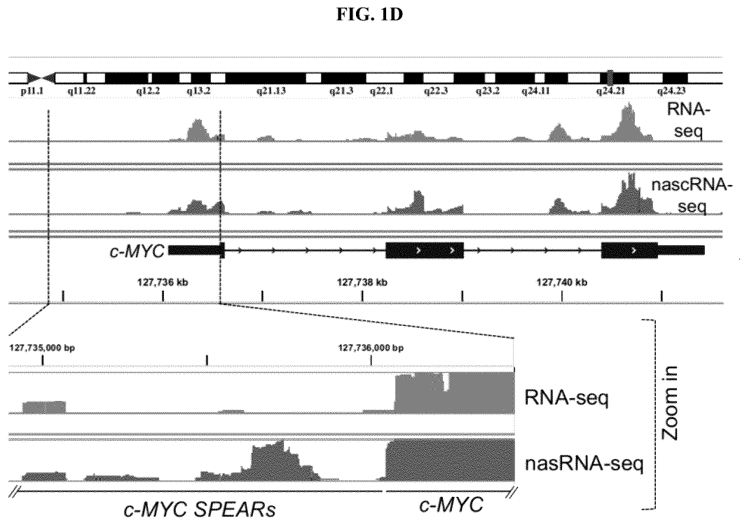

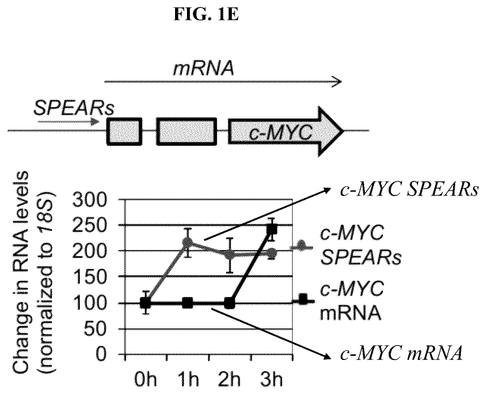

The experiments of this example investigated whether the epigenetic balance between acetylated and unmodified forms of H2A.Z are maintained by locally induced ncRNAs of a type similar to DNMT1-interacting RNAs (“DiRs”), which control cell type-specific DNA methylation patterns. Mapping RNAs arising from the CCAAT/enhancer-binding protein alpha (“CEBPA”) locus led to a definition of a transcript upstream of the CEBPA DiR (ecCEBPA), termed Upper Transcript (UpTr) (FIG. 1A). Expression of UpTr precedes both that of ecCEBPA and the CEBPA mRNA during Early S-Phase (FIG. 1A). Since the majority of active genes undergo replication in early S phase, which is followed by formation of the appropriate active chromatin, the experiments of this example investigated whether UpTr (and similar transcripts from other loci) control expression of the respective mRNAs by playing a role in the reassembly of the chromatin.

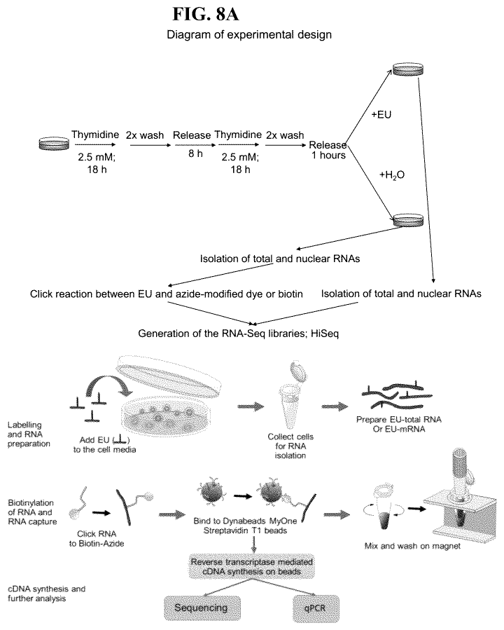

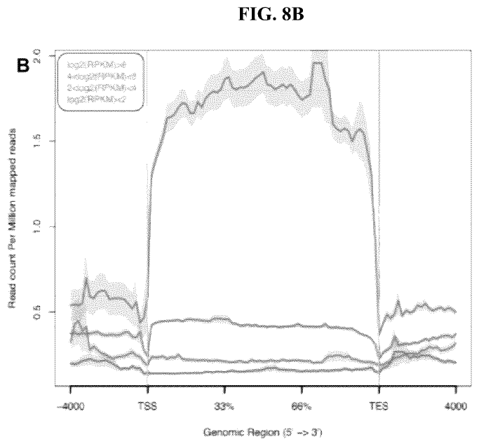

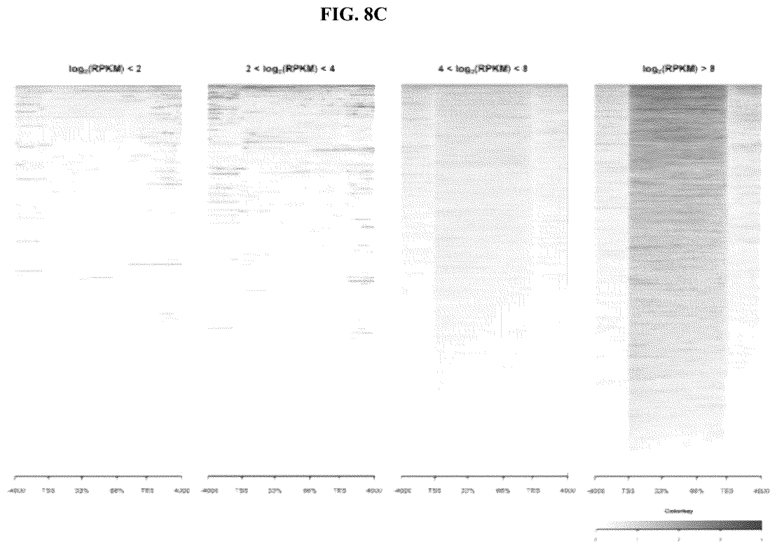

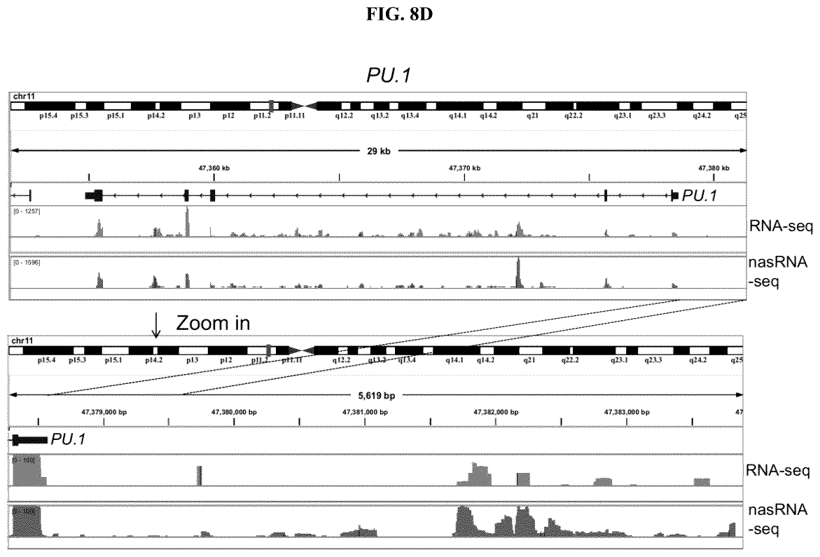

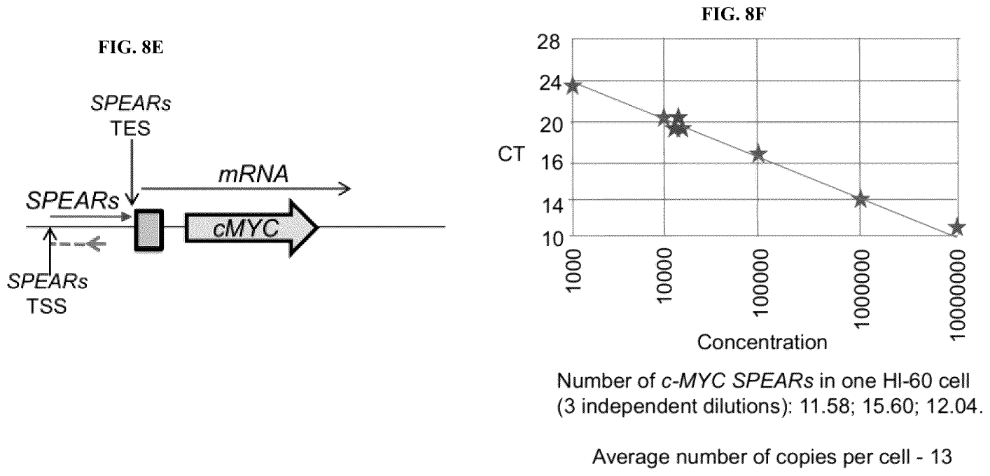

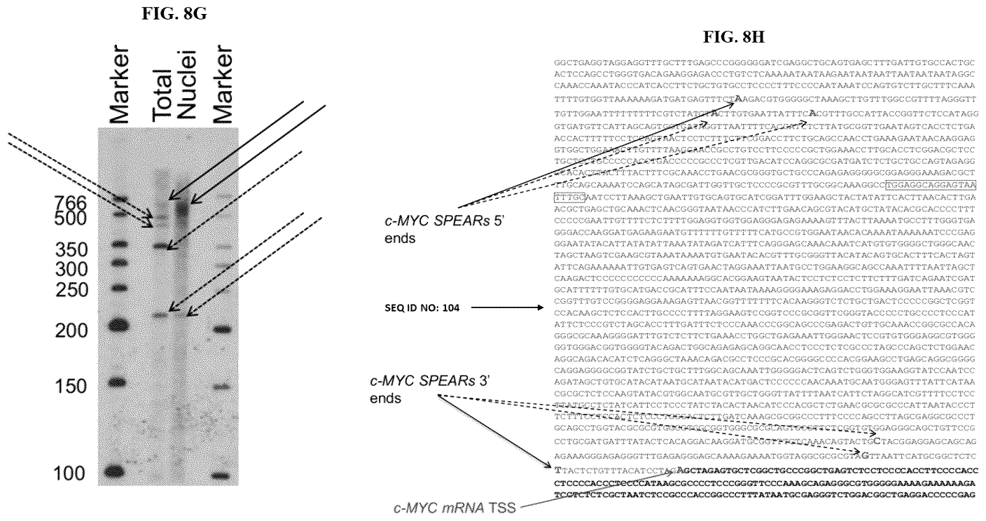

To establish the global occurrence of similar newly minted RNAs, which are disclosed herein as “S-Phase Early RNAs” (“SPEARs”), nascent RNAs were captured and sequenced (nasRNA-seq). Synchronized human HL-60 cells were labeled with the ribonucleotide homolog 5-ethynyl uridine (EU) for one hour upon release into S phase. The collected RNAs were then biotinylated by click chemistry, isolated on streptavidin beads and deep-sequenced to produce a nasRNAs library (FIG. 8A, and FIG. 1B). Four distinct groups of SPEARs were ranked by level of expression (FIG. 8A, FIG. 8B, FIG. 8C). Analysis of transcripts from all four groups revealed that the SPEARs initiate close to the transcription start sites (TSS) of coding genes and correlate with their expression levels (FIG. 1C, FIG. 8B, and FIG. 8C).

Among SPEARs-regulated genes, c-MYC was identified, which is the oncogene most frequently altered in cancer. Examples of SPEARs arising from the c-MYC locus (“c-MYC SPEARs”) and from the PU.1 locus (“PU1 SPEARs”) are shown in FIG. 1D and FIG. 8D, respectively. c-MYC SPEARs demonstrated an expression pattern similar to UpTr (FIG. 1E), and were shown to be represented by about −13 copies in the nucleus of HL-60 cells (FIG. 8E and FIG. 8F). They were mapped by primer extension and 5′, 3′-RACE (FIG. 8E, FIG. 8G, and FIG. 8H).

### Example 2: SPEARs Interact with H2A.Z, acH2A.Z and TIP60

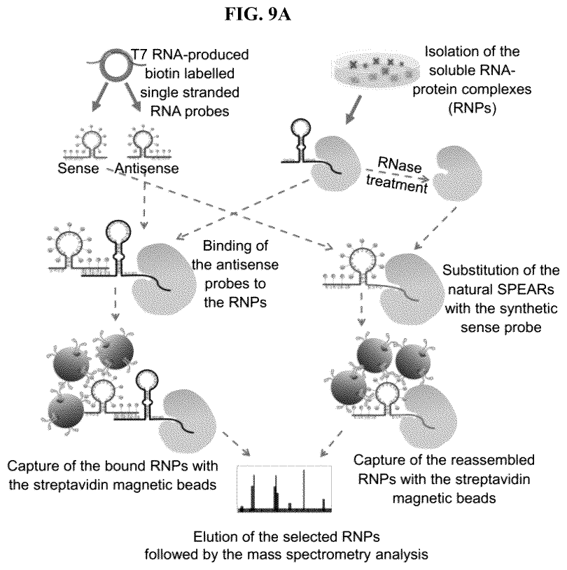

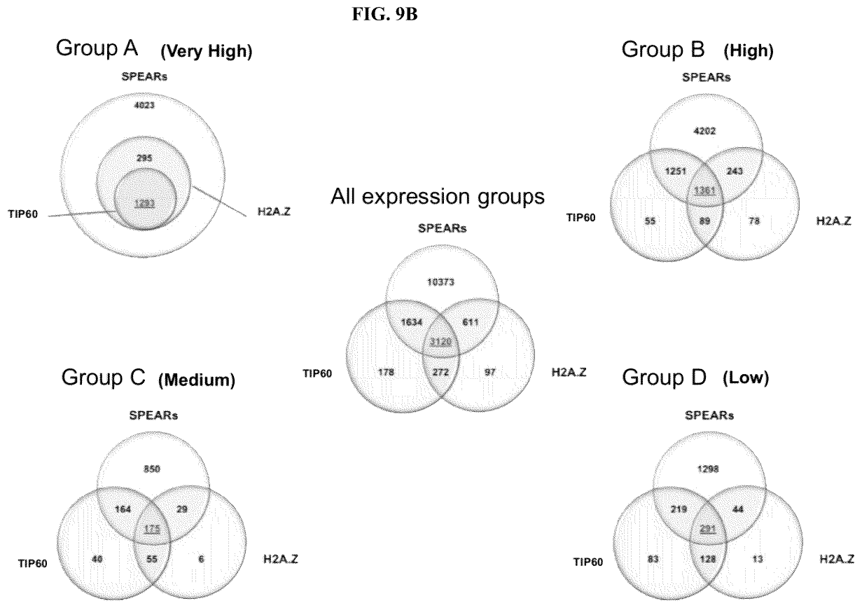

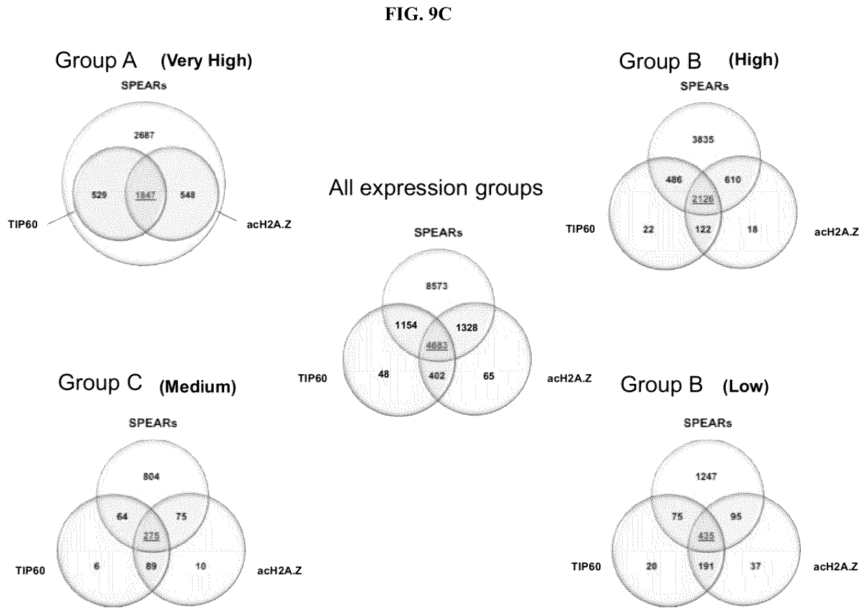

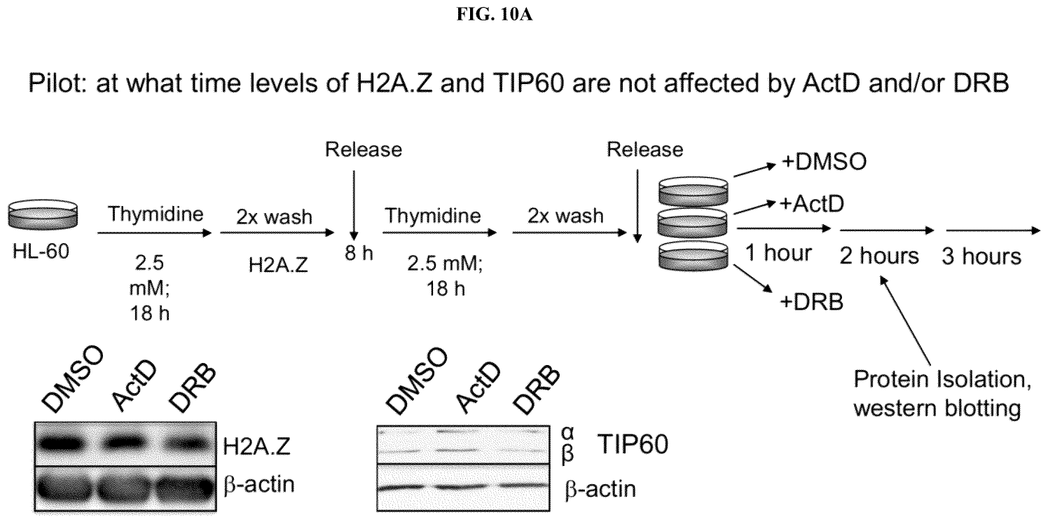

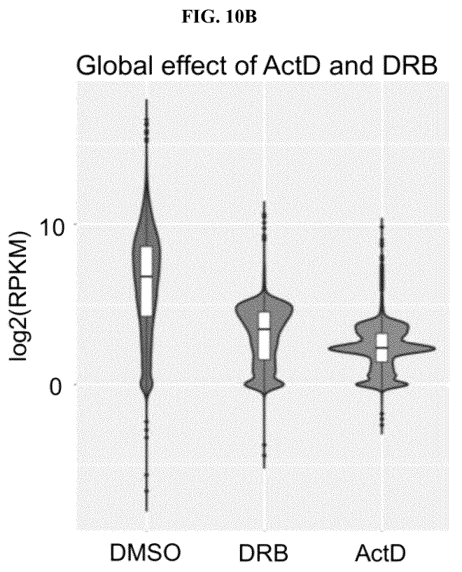

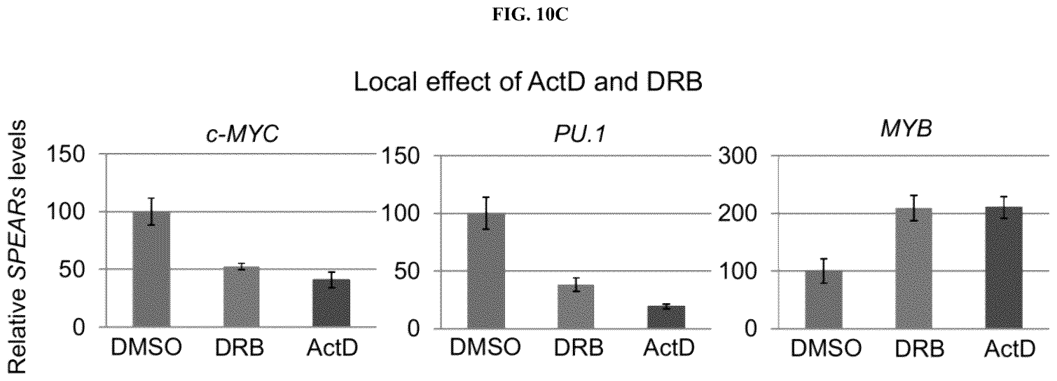

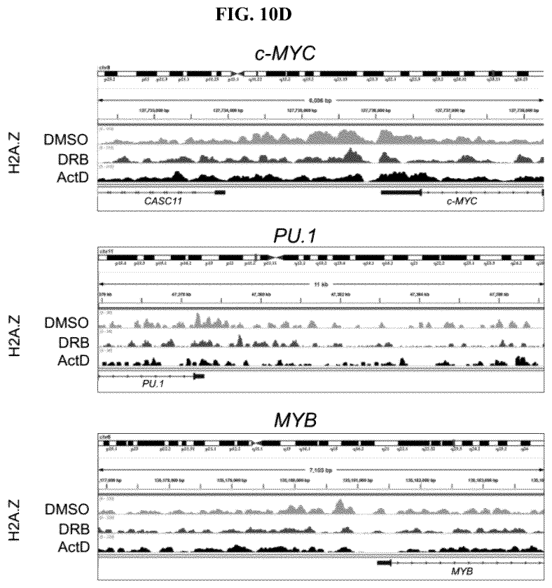

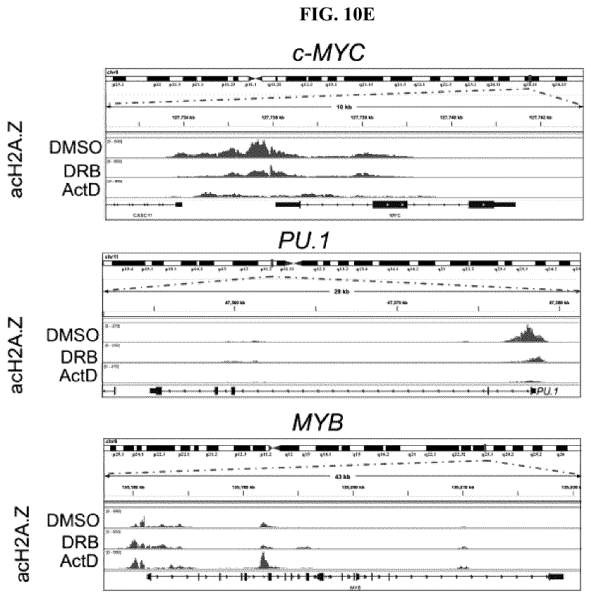

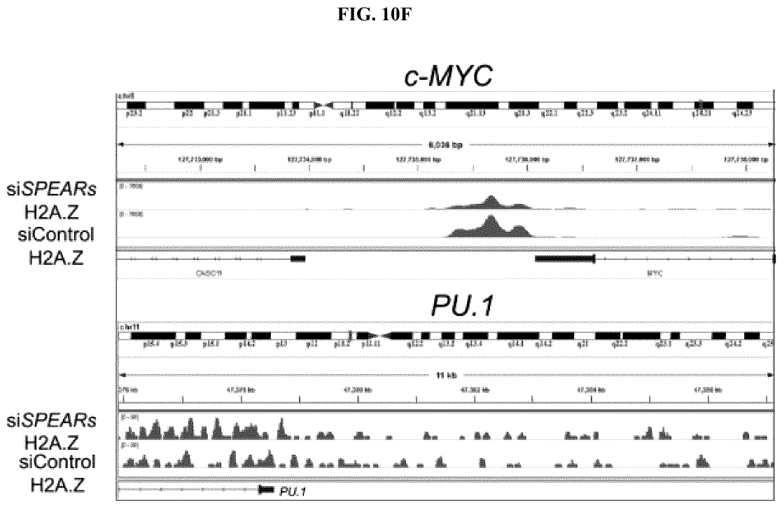

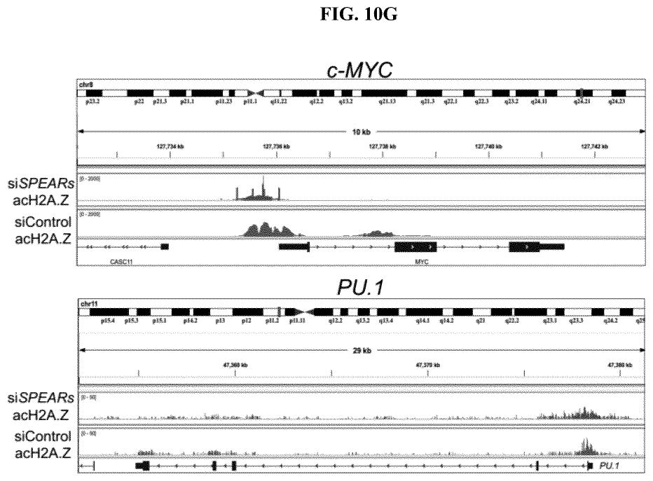

Given the close proximity of SPEARs to TSS, the experiments of this example analyzed the possibilities of a link between the expression of SPEARs and H2A.Z/acH2A.Z, which are a variant histone having the acetylation mark. This link was investigated by: (i) Ribonucleoprotein (RNP) pull-down experiments followed by mass spectrometry; and (ii) RNA immunoprecipitations followed by RNA sequencing (RIP-Seq). The sequences of five SPEARs identified by nasRNA-Seq (FIGS. 8A-8H), and corresponding to gene loci c-MYC, PU.1, MYB, CEBPA, and CTCF, were verified by primer extension and 5′3′ RACE (FIG. 8G and FIG. 8H). The approximately 500 nt long uninterrupted sequence segments (FIG. 9A, FIG. 9B, and FIG. 9C) were cloned under a T7 RNA polymerase promoter to express biotinylated sense and antisense SPEARs probes for RNP pull-down experiments. Probes from both strands can identify SPEARs-containing RNPs: antisense probes through direct base-pairing with the natural SPEARs and sense probes by replacing them (e.g., see FIG. 9A). Recovered RNPs interacting with both the antisense and sense SPEARs probes, and a negative control (D-Biotin), were analyzed by mass spectrometry. Among peptides pulled down by the SPEARs probes were several corresponding to H2A.FZ/H2A.FV (FIG. 10A, FIG. 10B, FIG. 10C, FIG. 10D, FIG. 10E, FIG. 10F, and FIG. 10G). It was not possible to distinguish the very homologous forms H2A.FZ vs. H2A.FV, and no peptides containing acetylation sites were detected, due to the relatively low fragment coverage. Other peptides pulled down by the SPEARs probes corresponded to histones H2A, H2B, H3, H4 and H1. No peptides corresponding to H2A/H2A.Z or other histones were detected by the negative control probe (FIG. 10A, FIG. 10B, FIG. 10C, FIG. 10D, FIG. 10E, FIG. 10F, and FIG. 10G).

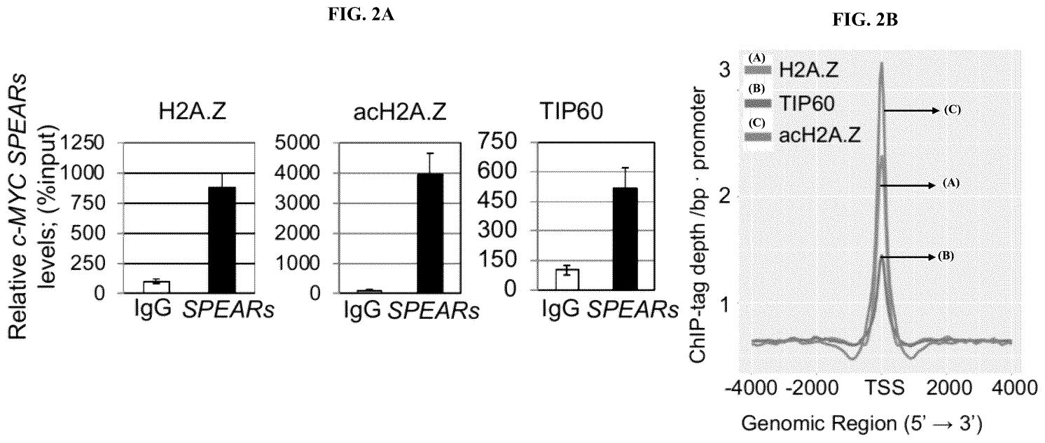

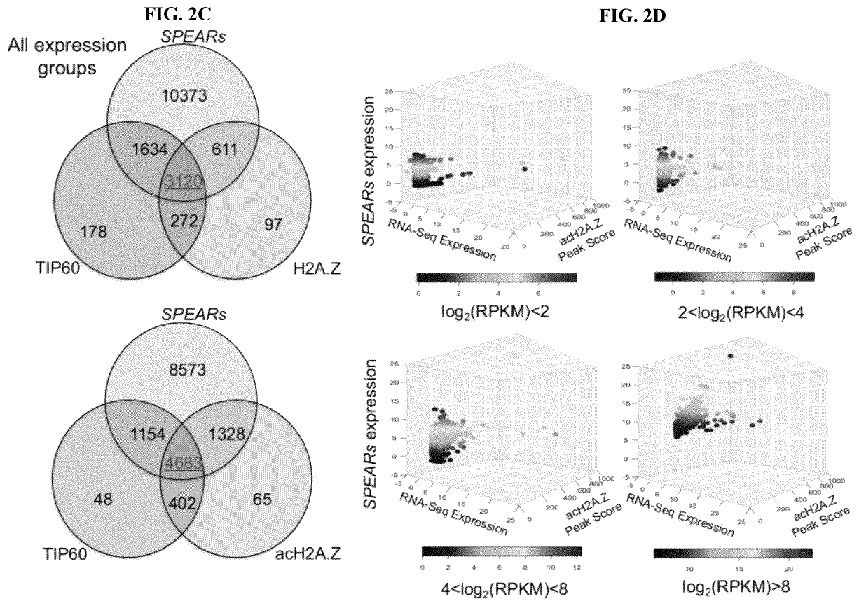

The experiments of this example also investigated whether SPEARs exert their function through a direct interaction with H2A.Z and histone acetyl-transferase (HAT) TIP60, an enzyme that acetylates H2A.Z. To directly test the interaction of SPEARs with H2A.Z, acH2A.Z and TIP60, RIP-Seq was performed using antibodies to H2A.Z (all forms), to acetylated H2A.Z (acH2A.Z) and to TIP60 (FIG. 2A). Importantly, a significant overlap of RIP-Seq peaks was observed in the immunoprecipitations using all three antibodies: considerable co-localization of the three data sets was observed in the regions around TSS (FIG. 2B). To estimate the magnitude of H2A.Z, acH2A.Z and TIP60 interaction with SPEARs, the nasRNA-Seq library was aligned with the TIP60, H2A.Z and acH2A.Z RIP-Seq databases (FIG. 2C, FIG. 2D, FIG. 9A, FIG. 9B). The overlap of 3120 loci for H2AZ and 4683 for acH2A.Z with expressed genes demonstrates a likely global involvement of SPEARs in cooperation with TIP60 and H2A.Z/acH2A.Z in establishing an active expression mode at the corresponding genes. Thus, these alignments demonstrate direct correlation of the SPEARs expression levels with those of the corresponding genes and, importantly, with the occupancy of acH2A.Z within the respective loci (FIG. 2D; axes “RNA-Seq Expression” and “acH2A.Z Peak Score”, respectively). Comparison of the H2A.Z and acH2A.Z data implies that it is the acetylated form of H2A.Z that is largely bound to the SPEARs.

Taken together, the experiments of this example, including the pull-downs and the RIP experiments, demonstrate a dual interaction of SPEARs with H2A.Z, acH2A.Z and with TIP60.

### Example 3: SPEARs Carry Common Binding Motifs

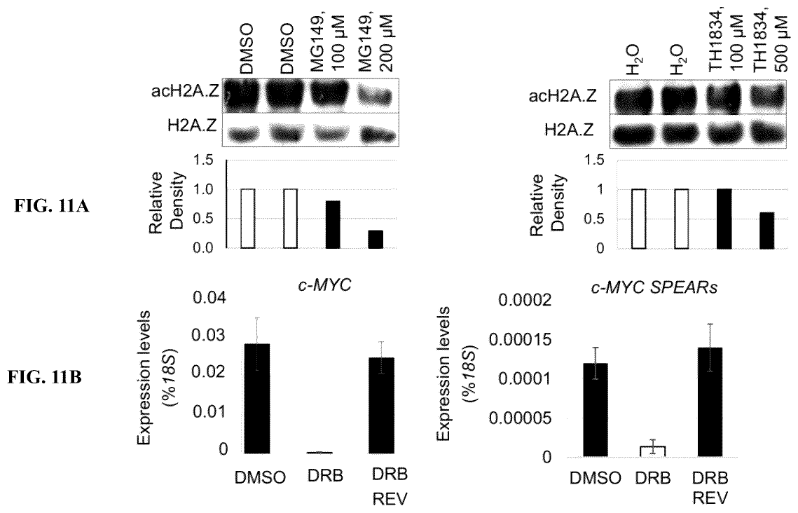

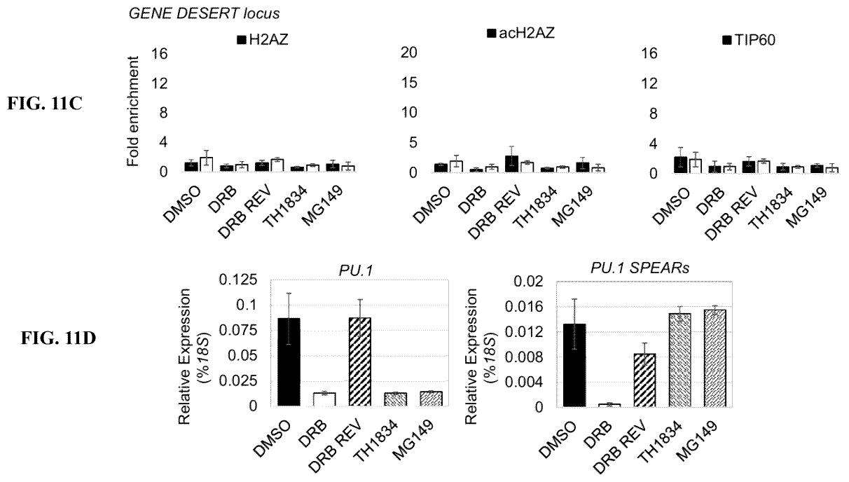

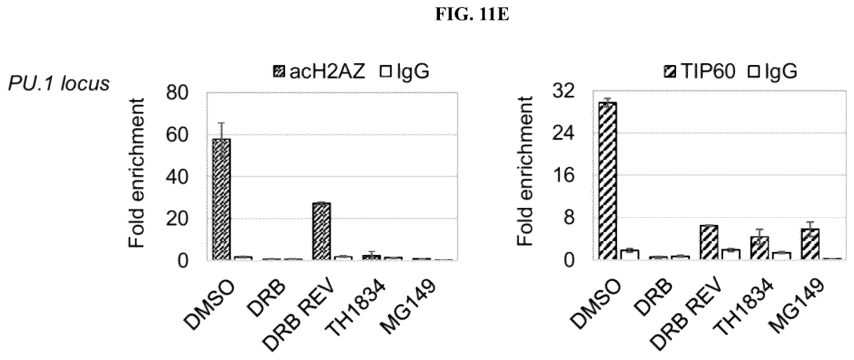

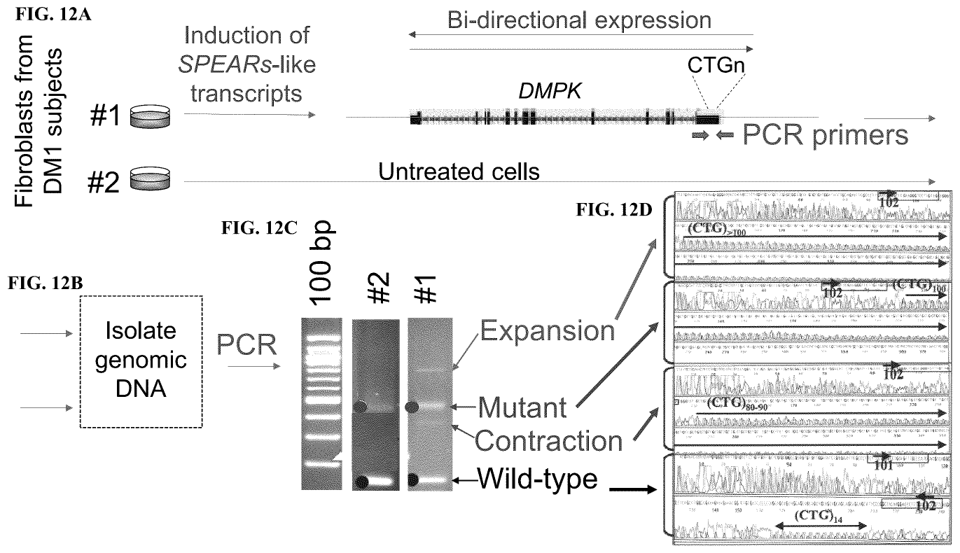

To further test the SPEARs-H2A.ZMTIP60 relationship, motif discovery analysis was performed on the SPEARs to look for common binding motifs. Briefly, about ˜14000 promoter loci (Broad HMM) were subjected to coverage calculation and filtered according to expression level. Prior to Motif Discovery analysis, 5′ and 3′ SPEARs boundaries were inferred from nasRNA-Seq and corresponding sequences retrieved (see “Methods” below for details; FIG. 3A). Twenty-five motif candidates were identified (FIG. 11A, FIG. 11B, FIG. 11C, FIG. 11D, FIG. 11E). Amid the potential candidates, three motifs: 3, 5, and 9 exhibited the most significant enrichment across the SPEARs-regulated gene loci. Importantly, no similar motifs were found within the largest classes of nuclear RNAs, i.e. transcripts arising from short and long interspersed nuclear elements (SINEs and LINEs), nor in ribosomal RNAs (see “Methods” below for details). Among the top three candidates, motif 9 (corresponding to the RNA oligonucleotide RM9A; FIG. 3B) was ranked as the strongest motif enriched in the c-MYC SPEARs sequence. This motif was enriched in both acH2A.Z and TIP60 RIP-Seq overlapping peaks (52% and 13%, respectively), located near the peak centers (FIG. 3C). In line with these results, RNA electrophoresis mobility shift assays (REMSAs) were used to show that RM9A is able to form RNPs with H2A.Z and with TIP60 in vitro, (FIG. 3D, lanes 3, 9 and 13; and FIG. 3E, lane 4). A shift in migration was observed after the incubation of RM9A with synthetic peptides corresponding to the N-terminal sequences of H2A.Z, both unmodified and acetylated at lysine 7 (K7) (FIG. 3D; right panel). Acetylation of the H2A.Z tail peptide did not abrogate the binding, thereby excluding the possibility that simple charge-charge interactions are responsible for SPEARs-H2A.Z or SPEARs-acH2A.Z complexes. Interestingly, the presence of a predicted stem-loop-like structure (RNAfold, FIG. 3F) seems to be required for RM9A binding to H2A.Z and TIP60 (FIG. 3D). Indeed, mutation of RM9A abolishing the predicted stem-loop structure, did change the strength and the mobility of the RNA-TIP60 complexes (Mut RM9A; FIG. 3E, lane 5). Unrelated oligonucleotides (UR1, UR2 and UR3) lacking the common binding motif did not form detectable RNPs complexes with TIP60/H2A.Z (FIG. 3D and FIG. 3E), thus supporting a strong element of both primary and secondary structural recognition between TIP60/H2A.Z and SPEARs.

To evaluate whether RNAs might be important for binding of remodeling complexes to chromatin (e.g., the ability of SPEARs to facilitate binding of TIP60 to the chromatin), TIP60 binding was compared to the RNA (single-stranded RM9A) and DNA (double-stranded DM9A) oligonucleotides of the same primary sequence. The results of the REMSA/EMSA demonstrated a stronger TIP60 binding to the RNAs than to the DNA (FIG. 3E; lane 4 vs. lane 6). Similar weak complex formation was observed between TIP60 and the DNA oligonucleotide duplexes (DM2 and DM3) of the same primary sequence as the RNA oligonucleotides (UR2 and UR3, respectively), presented in FIG. 3E, lanes 13 and 20.

The experiments of this example demonstrate that the binding between the TIP60 and the RNA relies on both primary and secondary structures as compared to TIP60 binding to DNAs.

In conclusion, these experimental results point to an interaction of SPEARs with the histone acetyltransferase TIP60 and histone H2A.Z, in particular its modified form acH2A.Z.

### Example 4: SPEARs are Involved in H2A.Z Acetylation and Exchange

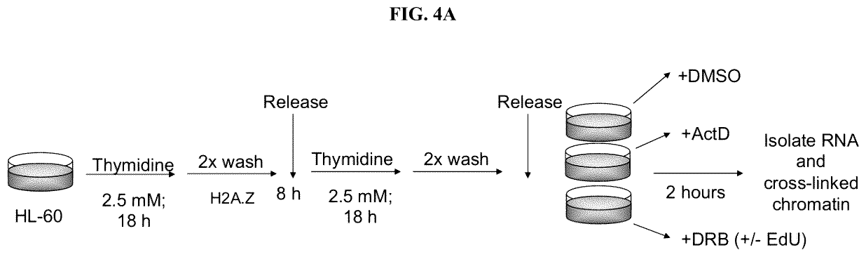

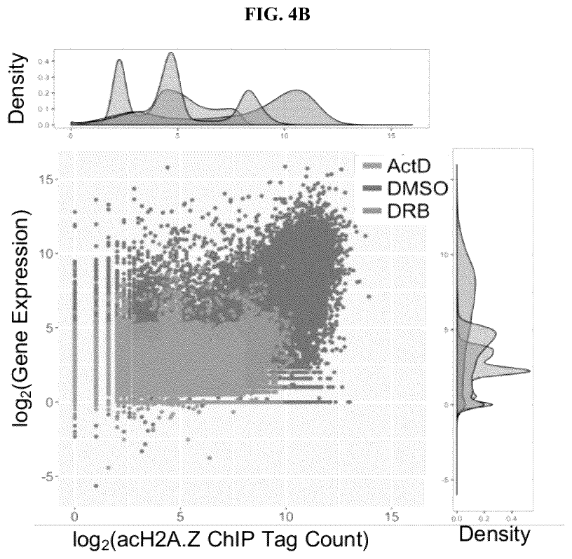

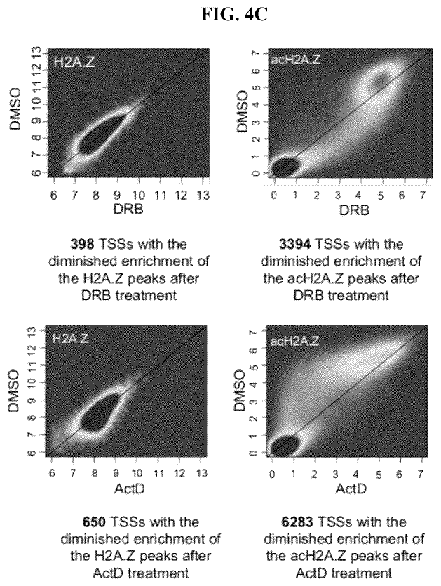

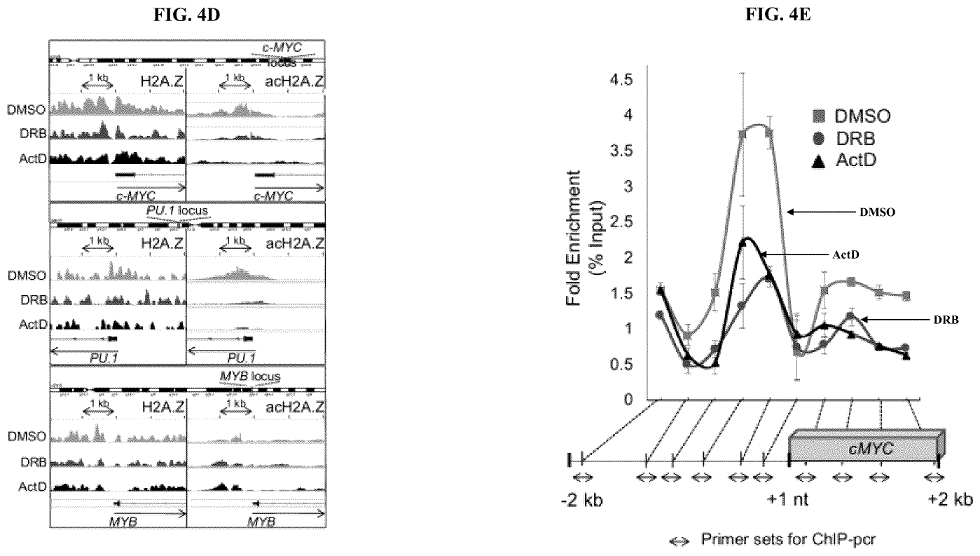

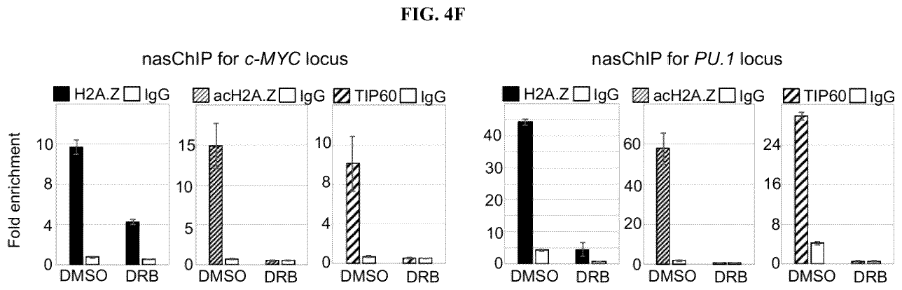

To directly examine whether SPEARs are required for the deposition of acH2A.Z at the TSS of their corresponding gene, transcription was pharmacologically inhibited followed by chromatin immunoprecipitation (ChIP) with the H2A.Z and acH2A.Z antibodies (e.g., see FIG. 4A). Two transcription inhibitors, Actinomycin D (ActD; RNA Polymerase I, II and III inhibitor) and 5,6-Dichlorobenzimidazole 1-β-D-ribofuranoside (DRB; RNAPII Inhibitor), were used at concentrations sufficient to block both RNA Polymerases II and III. Cells were synchronized with a double thymidine block and released into S Phase with ActD/DRB added. The rationale behind the use of ActD and DRB is: (i) to globally assess the effect of SPEARs suppression over a short time frame; (ii) to test effects resulting from the two different pathways triggered by ActD and DRB; and (iii) to take advantage of the reversibility of DRB treatment. Cells were treated with the inhibitors for two hours, an interval during which the overall levels of H2A.Z and TIP60 proteins were not affected (FIG. 10A), while the global expression levels of both the coding genes and the SPEARs were significantly decreased (FIG. 10B and FIG. 10C). Two hours after drug treatment, cells were crosslinked and subjected to ChIP-Seq analyses. FIG. 4B demonstrates that loci with suppressed expression of SPEARs showed diminished enrichment in acH2A.Z, indicating that SPEARs are involved in the precise placement of this epigenetic mark. Relative changes were then investigated in the footprints of unmodified H2A.Z and acH2A.Z in the vicinity of TSSs that result from DRB- and ActD-induced inhibition of SPEARs. The enrichments of ChIP-Seq signals for H2A.Z and acH2A.Z around 38,512 TSS (defined in the hg38 UCSC canonical gene table) following drug treatment were then compared with their occupancy in mock-treated (DMSO) cells. In these experiments, it was a drastic reduction was observed in acH2A.Z enrichment at the TSS of samples treated with either DRB or ActD, in contrast to only modest changes in the occupancy levels of the unmodified H2A.Z (FIG. 4C; see “Methods” below for details). Cells treated with DRB showed a drop of at least a 2-fold in acH2A.Z occupancy for 3,394 TSS when compared to cells without the treatment, while only 398 TSS present a similar trend for total H2A.Z signal. ActD-treated cells followed this pattern with a greater than 2-fold decrease in the levels of acH2A.Z at 6,283 TSS compared to untreated samples. In contrast, 650 TSSs exhibited a comparable decrease in the occupancy of total H2A.Z. The observation of a decrease 10 times greater in acH2A.Z compared to H2A.Z in treated samples, suggested the involvement of SPEARs in the maintenance of normal levels of H2A.Z acetylation at TSS, i.e. to the generation of the active epigenetic mark acH2A.Z. FIG. 4D (upper and middle panels) depicts examples of combined snapshots of two individual loci, c-MYC and PU.1, both giving rise to SPEARs negatively affected by ActD and DRB (shown in FIG. 10C), demonstrating a decrease in the intensity of H2A.Z and acH2A.Z peaks, i.e., the more pronounced being the decrease of acH2A.Z levels (full snapshots are shown in (FIG. 10D and FIG. 10E). For the c-MYC and PU1 loci, respectively, the ratios of control (DMSO) to drug treated H2A.Z peak intensities were: [DMSO/DRB/ActD]^{c-MYC}=1/0.76/0.67 and [DMSO/DRB/ActD]^{PU.1}=1/0.87/0.82; and the ratios of control (DMSO) to drug treated acH2A.Z peak intensities were: [DMSO/DRB/ActD]^{c-MYC}=1/0.62/0.36 and [DMSO/DRB/ActD]^{PU.1}=1.0/0.25/0.13. These results were confirmed by quantitative ChIP-PCR for the c-MYC locus (FIG. 4E). By contrast, loci escaping the down-regulating effects of the drugs on their SPEARs exhibited an acH2A.Z intensity ratio concordant with the change in SPEARs levels. For example, the MYB locus, gives rise to SPEARs positively affected by ActD and DRB (FIG. 10C), and exhibits an increase in the intensity of the acH2A.Z peaks, with no significant changes of the intensity of the unmodified H2A.Z peaks (see FIG. 4D, bottom panel; and FIG. 10D and FIG. 10E). For MYB the ratios of control (DMSO) to drug treated acH2A.Z peak intensities are: [DMSO/DRB/ActD]^{MYB}=0.62/0.89/1.0, which corresponds to the increases in its SPEARs levels (FIG. 10C). Thus, unexpectedly, for a locus where expression of SPEARs is positively affected by ActD/DRB, the experiments in this example illustrate how an increase in SPEARs levels leads to a corresponding rise in the intensity of the acH2A.Z peaks.

To confirm the results of the ChIP-Seq, and to track the deposition of the H2A.Z, acH2A.Z nascent chromatin immunoprecipitation (nasChIP-PCR) was performed with the antibodies to H2A.Z, acH2A.Z and TIP60, as nasChIP-PCR can be used to track histone deposition. Cells were synchronized with a double thymidine block, released into S Phase and treated with DRB for 2 hrs. The medium was supplemented with the EdU DNA analog to enable collection of nascent chromatin DNA (FIG. 4A). During the inhibition of transcription with DRB, the c-MYC locus showed diminished enrichment in H2A.Z, acH2A.Z and TIP60 (FIG. 4F, 3 left panels). The results of these experiments provide an important link between the suppressed c-MYC SPEARs expression and the reduced levels of the replacement histone H2A.Z, as an even greater degree of the loss of the acetylated acH2A.Z forms at the TSS of the c-MYC locus. Similar results were obtained for another gene locus, PU1 (FIG. 4F, right panels).

Taken together, the results of the ChIP-Seq and nasChIP-PCR experiments define the role of the transcription in deposition of the replacement histone H2A.Z. The more robust drop of the acetylation level of the H2A.Z, as compared to the decrease of the unmodified form at the TSSs of the genes, as well as loss of the TIP60 at the same sites, points to the role of transcription in mediating the acetylation of H2A.Z.

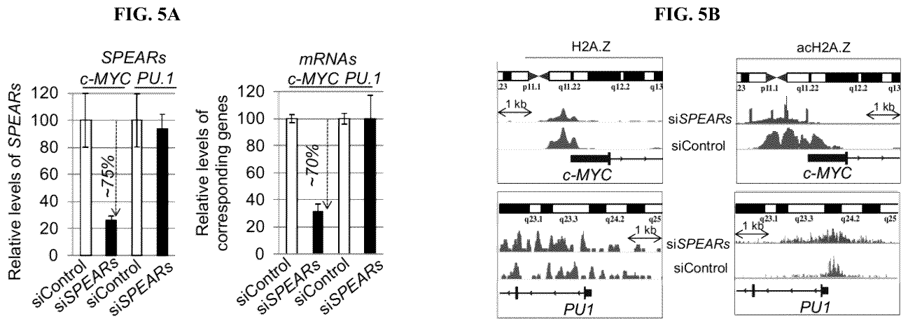

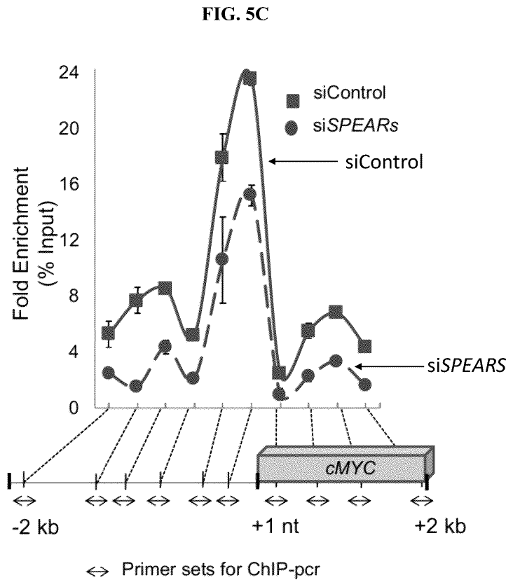

In a reverse experiment, RNAi-mediated downregulation of specific SPEARs was tested followed by ChIP-Seq and ChIP-qPCR analyses, to see whether RNAi-mediated downregulation led to a lowering of H2A.Z and acH2A.Z levels at the TSS of targeted loci and in reduced expression of the corresponding gene. Comparison of the two panels of FIG. 5A demonstrates that reduction of c-MYC SPEARs by ˜75% leads to a decrease of c-MYC mRNA expression of similar magnitude (˜70%). In contrast, the unaffected control PU1 SPEARs is accompanied by essentially no change in PU1 mRNA levels. FIG. 5B presents snapshots of H2A.Z and acH2A.Z levels at the targeted and control loci, c-MYC and PU1, respectively (full snapshots in FIG. 10F and FIG. 10G). Knockdown of the c-MYCSPEARs was associated with a significant decrease of acH2A.Z levels at the TSS of the c-MYC gene as compared to the TSS of the non-targeted control PU1 gene. The results of the RNAi/ChIP experiments demonstrate that expression of the c-MYCSPEARs is linked to the level of H2A.Z acetylation at the c-MYC TSS. These experimental results demonstrate the causality between the expression of SPEARs and the deposition of the activating histone mark, resulting in the transcriptional activation of the adjacent gene. FIG. 5C shows a verification of the acH2A.Z ChIP-Seq analysis using quantitative ChIP-PCR for the c-MYC locus.

Overall, downregulation of SPEARs using independent pharmacological and RNAi-induced methods led to a substantial reduction in the activating epigenetic mark acH2A.Z. Loss of the acetylated form of H2A.Z from chromatin in the vicinity of active TSS implies a reduction of its native activity and, consequently, of expression of the respective gene (FIG. 5A; right panel).

### Example 5: SPEARs Regulate the Expression of the Respective mRNA Via TIP60/acH2AZ Recruitment/Deposition

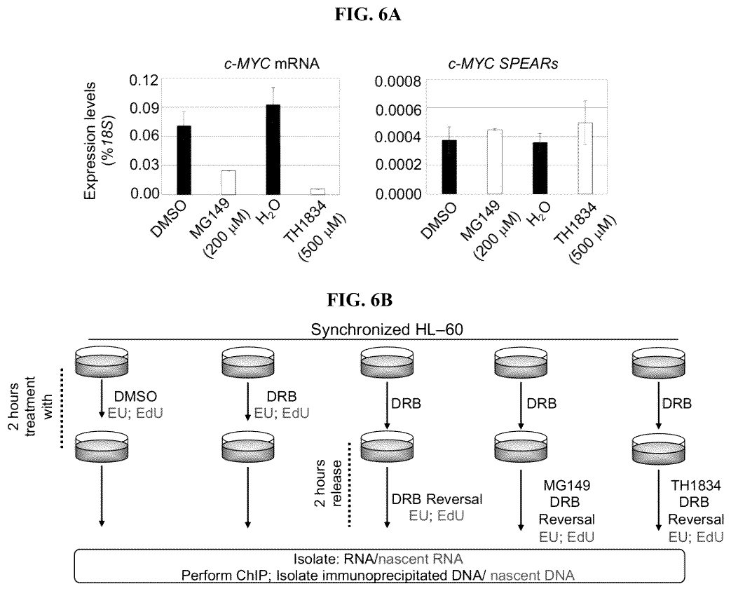

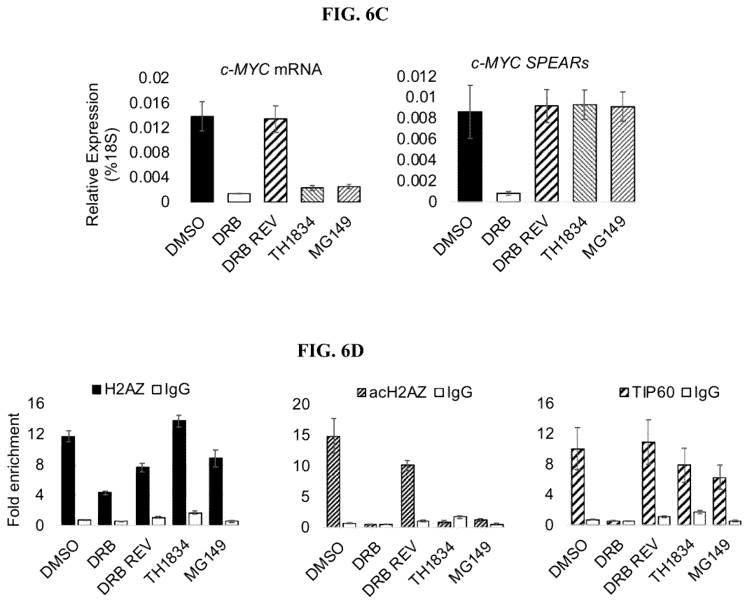

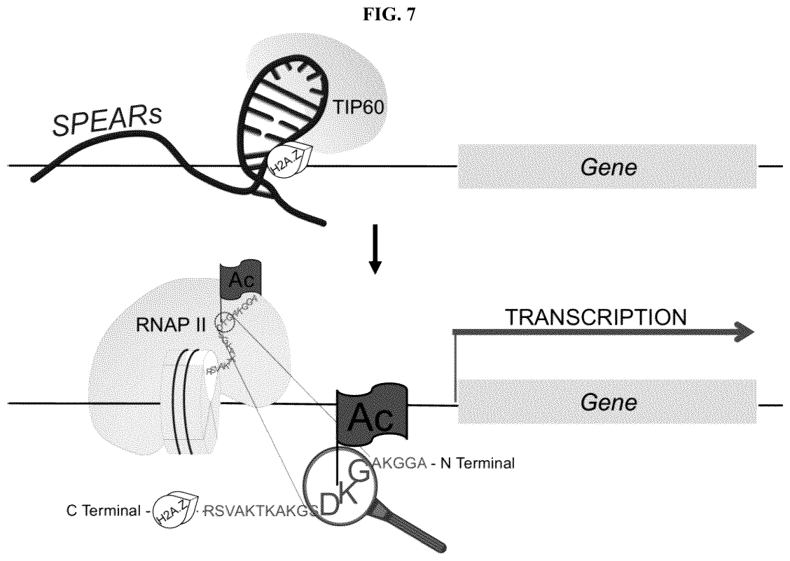

To demonstrate that the SPEARs-mediated regulation of corresponding mRNAs executes through the TIP60/acH2AZ pathway, the effects of TIP60 inhibition on the c-MYC locus were tested in this example. Two TIP60/HAT inhibitors, TH1834 and MG-149, were used in HL60 cells to reduce the total level of H2A.Z acetylation (FIG. 11A). This led to significant downregulation of mRNAs, in contrast to almost unchanged levels of SPEARs transcripts (FIG. 6A). To examine the possibility of a direct regulation of c-MYC expression by TIP60 and acH2A.Z interacting with the SPEARs, the regulating effect of nascent SPEARs on the level of nascent mRNA expression in the presence or absence of the two TIP60 inhibitors was monitored, taking advantage of the reversibility of the DRB transcriptional inhibitor. The rationale behind this approach was to analyze only transcripts re-appearing after the release from the transcriptional block during the HAT inhibition. The short time of the treatment should rule out any secondary effects of HAT inhibition.

Cells were synchronized with a double thymidine block and released into S Phase with DMSO/DRB added. After 2 hours (during which H2A.Z levels remained unaffected, but SPEARs and mRNA levels drop, see FIG. 10A), cells were washed and then incubated for a further 2 hours with or without the TIP60 inhibitors. This interval is sufficient for the levels of both the c-MYC mRNAs and its SPEARs to be restored when the TIP60 inhibitors are absent (results shown in FIG. 11B). The medium was supplemented with the EU RNA and EdU DNA analogs to enable collection of nascent RNAs and newly formed chromatin that had escaped the drug-induced inhibition of transcription or acetylation. After 2 hours with the TIP60 inhibitors present (MG-149 at 200 μM and TH1834 at 500 μM), during which the levels of acH2A.Z dropped (see FIG. 11A), cells were crosslinked and subjected to nasChIP and nascent RNA expression analyses (nas-qRT-PCR) (FIG. 6B). Collected RNAs were biotinylated by click chemistry, isolated on streptavidin beads and analyzed by nas-qRT-PCR (see “Methods” below for details; FIG. 8A; bottom panel). The results in FIG. 6C show that the correlation between the expression of the SPEARs and the mRNA no longer holds when TIP60 activity is inhibited, i.e. the restored levels of the SPEARs are incapable of rescuing the expression of c-MYC mRNA to the level defined by the reversed DRB. These data suggest that the function of SPEARs to regulate c-MYC mRNA expression is impeded by inhibition of the HAT activity of the TIP60.

To further confirm that SPEARs realize their c-MYC regulatory function directly through the TIP60/acH2A.Z pathway, the differences in chromatin occupancy of H2A.Z, acH2A.Z and TIP60 were assessed after inhibition of transcription (“DRB”), reversal of inhibition (“DRB REV”) and reversal of inhibition in the presence of the two TIP60 inhibitors, using nasChIP-qPCR. In particular, it is important to check whether the drop in c-MYC expression after treating the cells with DRB and TIP60 inhibitors is correlated with diminished enrichment in TIP60, acH2A.Z or H2A.Z. Nascent DNA was isolated from the chromatin immuno-precipitated with antibodies to H2A.Z, or acH2A.Z or TIP60, biotinylated by click chemistry, then isolated on streptavidin beads (see “Methods” below for details), and finally analyzed by qPCR at amplicons corresponding to maximum enrichment within the c-MYC locus (FIG. 4E). By analyzing the nascent RNAs and chromatin, only the immediate changes in c-MYC expression and TIP60, acH2A.Z and H2A.Z occupancy were examined within the locus as imposed by the inhibition of transcription and/or acetylation. The middle and right panels of FIG. 6D demonstrate that the c-MYC locus with suppressed expression of its SPEARs shows diminished enrichment in both TIP60 and acH2A.Z and the reversal of the transcription inhibition led to the full restoration of the enrichment of both TIP60 and acH2A.Z. This indicates that the SPEARs are involved in the recruitment of TIP60 and precise placement of the epigenetic acetylation mark. When the two HAT inhibitors are present, the reversal of transcriptional inhibition can only restore the enrichment levels of the unmodified H2A.Z and TIP60 (FIG. 6D; left and right panels), but not the levels of acH2A.Z (middle panel). In contrast, no great changes were observed for the distribution of unmodified H2A.Z (FIG. 6D; left panel). Furthermore, no enrichments of H2A.Z, acH2A.Z or TIP60 was detected within the Gene Desert locus (FIG. 11C). Given the fact that local (in the vicinity of the c-MYC locus TSS; FIG. 6D; left panel), as well as overall levels of H2A.Z were unchanged throughout the experiment (FIG. 11A), these results establish the leading role of the TIP60/acH2A.Z pathway in c-MYC SPEARs-mediated regulation of its corresponding gene, c-MYC. Similar results were obtained for the PU.1 locus (FIG. 11D and FIG. 11E). These findings using nasChIP are in line with the general trend obtained globally by ChIP-Seq. (FIG. 4C).

Collectively, the results shown in these experiments and examples demonstrate that the TIP60/acH2A.Z pathway is a general mechanism in SPEARs-mediated regulation of their corresponding adjacent coding genes.

### Example 6: Controlling the Direction of Replication Using SPEARs

In the experiments of this example, SPEARs are shown to control the direction of replication. As shown in the experiments of this example, by changing the direction of replication the expansion of trinucleotide repeats can be reversed. This is significant for the treatment of trinucleotide repeat disorders (“TRD”), which are caused by trinucleotide repeat expansion. Trinucleotide repeat expansion is a mutation wherein repeats of three nucleotides increase in copy numbers until a level is reached that results in instability of gene expression. In this example, SPEARs are shown to reverse the expansion of trinucleotide repeats, thereby showing that SPEARs are capable of treating trinucleotide repeat disorders. FIG. 12A (without wishing to be bound by theory), FIG. 12B, FIG. 12C, and FIG. 12D, are images showing how the induction of SPEARs-like transcription affects the size of trinucleotide repeats. From these experiments, one or more SPEARs can be overexpressed to regulate the direction of replication, and one or more SPEARs' inhibitors is capable of eradicating the cause of Trinucleotide repeat disorders (“TRDs”; e.g., Huntington's disease (HD), spinocerebellar ataxias, a movement disorder, autism) by reversing the expansion of trinucleotide repeats (“TNRs”, including CAG, CTG, CGG, and GAA) that occurs during replication and repair.

### Methods

### Mammalian Cell Culture

The human leukemia cell line HL-60 was obtained from ATCC and grown in glutamine containing medium in the absence of antibiotics, at 37° C. in a humidified atmosphere with 5% CO2.

Antibodies Used Rabbit polyclonal to human Histone H2A.Z-ChIP Grade (1:2,000; Abcam ab4174), Sheep polyclonal to human H2A.Z (K4+K7+K11) (1:2,000; Abcam ab18262), Rabbit polyclonal to human TIP60 antibody.

### RNA Isolation

Total RNA isolation was carried out and all RNA samples used in this study were treated with DNase I (10 U of DNase I per 3 μg of total RNA; 37° C. for one hour; in the presence of RNase inhibitor). After DNase I treatment, RNA samples were extracted with acidic phenol (pH 4.3) to eliminate any remaining traces of DNA. cDNA syntheses were performed with Random Primers (Invitrogen) or gene-specific primers with Transcriptor Reverse Transcriptase (Roche Applied Science) according to the manufacturer's recommendation. cDNA was purified with a High Pure PCR Product Purification Kit (Roche Applied Science).

qRT-PCR

Sybr green reaction was performed using iQ Sybr Green supermix (Biorad, Hercules, CA) using the following parameters: 95° C. (10 min), 40 cycles of 95° C. (15 s) and 60° C. (1 min) 72° C. (1 min). TaqMan analysis was performed using Hotstart Probe One-step qRT-PCR master mix (USB) at the following conditions: 50° C. (10 min.), 95° C. (2 min.), and then 40 cycles of 95° C. (15 sec.) and 60° C. (60 sec.).

Primers used for TaqMan real time PCR: Human MYC mRNA: ABI Cat. #Hs00153408_m1 Human MYB mRNA: ABI Cat. #Hs00920556_m1, Human IRF2BP2 mRNA: ABI Cat. #Hs00766250_g1, Human RRM2 mRNA: ABI Cat. #Hs00357247_g, Human CTCF mRNA: ABI Cat. #Hs00902008_m1, rRNA: ABI Cat. #4310893E, Human SS rRNA: ABI Cat. #Hs02385257_g1.

Primers used for strand-specific real-time RT PCR (Sybr): Reverse Transcriptase primer for c-MYC SPEARs: 5′-AAC CGC ATC CTT GTC CTG TGA GTA-3′ (SEQ ID NO: 1); PCR primers: Forward: 5′-ACA GGC AGA CAC ATC TCA GGG CTA-3′ (SEQ ID NO: 2); Reverse: 5′-ATA GGG AGG AAT GAT AGA GGC ATA-3′ (SEQ ID NO: 3); and Reverse Transcriptase primer for PU1 SPEARs: 5′-GGC TTT TGC TCT AAC CCA AC-3′ (SEQ ID NO: 4); PCR primers: Forward: 5′-ACT ATG CTG AAG ACC CTA CAC-3′ (SEQ ID NO: 5); Reverse: 5′-GCT CTA ACC CAA CAA ATG CC-3′ (SEQ ID NO: 6).

Nascent RNA/DNA capture was performed using Click-iT Nascent RNA Capture Kit (ThermoFisher) according to the manufacturer's instructions with minor modifications. Briefly, 1. Labeling the cells with EU/EdU. 200 mM EU or 30 mM EdU stock solutions were added to the cells, to a final concentration 0.5 mM or 30 μM, respectively. 2. Incubation. The cells were incubated for 1 or 2 hours. 3. RNA/DNA isolation. The cells were harvested and the RNA/DNA were isolated and dissolved in 14 μL of H₂O. 4. Biotinylation of RNA/DNA by Click reaction. Click-iT reaction cocktail (50 μL per reaction) was prepared accordingly to manufacturer's instructions: a mixture containing 1× Click-iT EU buffer; 2 mM CuSO₄; 1 mM Biotin azide; 13.25 μL of the isolated RNA; 10 mM Click-iT reaction buffer additive 1; 12 mM Click-iT reaction buffer additive 2 was prepared. After adding each component, the reaction cocktail was gently mixed by vortexing. The addition of the Click-iT reaction buffer additive 1 stock initiates the click reaction between the EU-RNA/EdU-DNA and biotin azide. Afterwards the Click-iT® reaction buffer additive 2 is added and incubated for 30 minutes with gentle vortexing. 5. RNA/DNA precipitation. 1 μL of ULTRAPURE™ Glycogen, 55 μL of 7 M ammonium acetate, and 750 μL of chilled 100% ethanol were added to the click reaction, incubated at −70° C. for at least 30 minutes and after centrifugation the pellet was dissolved in 125 μL of H₂O. 6. Binding biotinylated RNA/DNA to Dynabeads® MYONE™ Streptavidin Ti magnetic beads. The RNA/DNA binding reaction mixture included: 125 μL 2×Click-iT RNA binding buffer; 2 μL Ribonuclease Inhibitor or 2 μL of water for DNA; 125 μL of the isolated biotinylated RNA/DNA. The RNA binding reaction mixture was heated at 68-70° C. for 5 minutes and 50 μL of bead suspension added into the heated RNA binding reaction mixture. The tube containing the RNA/DNA binding reaction was incubated at r.t. for 30 min while gently vortexing to prevent the beads from settling. The beads were immobilized using the magnet and washed 5 times with 500 μL of Click-iT® reaction wash buffer 1 and 5 times with 500 μL of Click-iT® reaction wash buffer 2. Finally, the beads were resuspended in 50 μL of Click-iT reaction wash buffer 2 and the captured RNA immediately processed to cDNA synthesis. The captured DNA was released into 50 μL of boiling water and used in qPCR analyses.

Primer extension and 5′/3′ RACE cDNAs from the HL-60 cell line were synthesized as described above and run in urea-PAGE. 5′/3′ RACE was performed using the Exact START™ Eukaryotic mRNA 5′- & 3′-RACE Kit according to the manufacturer's instructions.

Double Thymidine block (early S-phase block) was carried out as described. Briefly, HL-60 cells were grown overnight to 70-80% confluence, washed twice with 1×PBS and cultured in DMEM (10% FCS)+2.5 mM Thymidine for 18 h (first block). Thymidine was washed out with 1×PBS and cells were grown in DMEM (10% FCS). After 8 hrs cells were cultured in the presence of thymidine for 18 h (second block) and then released as described. Synchrony was monitored by flow cytometry analysis of propidium iodide-stained cells using a LSRII flow cytometer (BD Biosciences) at the Harvard Stem Cell Institute/Beth Israel Deaconess Center flow cytometry facility.

DRB and Actinomycin D treatments were carried out as described. Briefly, after release from double thymidine block, HL-60 cells were treated with 100 μM of 5,6-Dichlorobenzimidazole 1-β-D-ribofuranoside (DRB) (Sigma Aldrich) or 0.8 μM of Actinomycin D (Sigma Aldrich) as indicated at each time point.

Down-Regulation of c-MYC SPEARs

27 siRNAs targeting the c-MYC SPEARs were designed and synthesized by siTOOLs Biotech as c-MYCSPEARs siPool (siSPEARs), the sequences are shown in the following Table 1. The siMyc were dissolved in nuclease-free water and used to transfect to HL-60 cells using Amaxa Cell Line Nucleofector Kit V, Program T-019 (Nucleofector II Device) according to the manufacturer's instructions. Briefly, 2×10⁶ cells/reaction were cultured in RPMI medium+10% FBS. The cells were collected by centrifugation. 1 μL siSPEARs and Negative Control siPool (siControl) was mixed with 4 μL nuclease-free water per reaction (siPool solution). Cells were resuspended in 100 μL room-temperature Nucleofector Solution per reaction and combined with the siPool solution. The final concentration of siSPEARs and siControl was 100 nM. The cells/siSPEARs and cells/siControl suspensions were transferred into certified cuvettes and taken through Nucleofector Program T-019 (Nucleofector II Device) in triplicates. 500 μL of the pre-incubated culture medium was immediately added to the cuvette and the samples were gently transferred into the wells of 6-well plate containing 500 μL of the pre-incubated culture medium. The samples were cultured for 24 hours, and then the electroporation repeated. The samples were cultured for another 24 hours. Live cells were harvested with Ficoll-Paque PLUS medium (GE Healthcare, #17144003), and RNA/chromatin extracted.

Short-hairpin RNAs Sequences are indicated in Table 1.

### Ribonucleoprotein (RNP) Fractionation

Equal number of viable cells (˜2 million), counted after Ficoll gradient purification, were used for each isolation. 1-5. Nuclei from 2×10⁶ cells were isolated, and briefly, equal amounts of viable cells were washed with ice-cold PBS supplemented with 5 mM vanadyl complex, 1 mM PMSF and resuspended in ice-cold lysis buffer: 1× Buffer A (10 mM HEPES-NaOH pH 7.6; 25 mM KCl; 0.15 mM spermine; 0.5 mM spermidine; 1 mM EDTA; 2 mM Na butyrate); 1.25 M sucrose; 10% glycerol; 5 mg/mL BSA; 0.5% NP-40; freshly supplemented with protease inhibitors (2 mM leupeptin, add as ×400; 2 mM pepstatin, add as ×400; 100 mM benzamidine, add as ×400; a protease inhibitor cocktail (Roche Applied Science, Cat. No. 1836153), 1 tablet/375 μL H₂O, add as ×100; 100 mM PMSF, add as ×100); 2 mM vanadyl complex (New England Biolabs); and 20 units/mL RNase inhibitor (RNAguard; Amersham Biosciences). Samples were incubated at 0° C. for ˜10 minutes and passed through several strokes in a Dounce homogenizer. The pelleted nuclei were resuspended in 0.5 ml lysis buffer and diluted with 2.25 mL Dilution Buffer (2.13 mL “Cushion” buffer plus 0.12 mL 0.1 g/mL BSA), freshly supplemented with protease inhibitors and overlaid onto 2 mL “cushions” (200 mL “Cushion” buffer consists of 15 mL ddH₂O; 15 mL 20× Buffer A; 30 mL glycerol; 240 mL 2.5 M sucrose; freshly supplemented with protease inhibitors) into one SW 55 Ti tube and centrifuged at 24,400 rpm, for 60 min at 4° C. Washed with PBS/1 mM PMSF. 7. Nuclei were then resuspended in 1.8 ml of cytoskeletal buffer (CSK-50:10 mM Pipes, pH 6.8; 300 mM sucrose; 50 mM NaCl; 3 mM MgCl₂; 1 mM EGTA; 5 mM vanadyl complex; 1 mM PMSF). 8. 20 μl (200 U) DNase I were added and incubation carried at 37° C. for 30 min and chilled on ice. 9. After addition of 200 μl of 2.5 M (NH₄)₂SO₄ (final 250 mM) the nuclei were incubated on a rocking platform 40 min at 4° C. and spun down at 2,000 g for 5 minutes at 4° C. 10. The supernatant fraction was discarded 11. The RNP-containing pellet was washed twice with ice-cold PBS and resuspended in 2 ml RIP buffer #4 (50 mM HEPES-KOH, pH 7.5, 100 mM NaCl, 1 mM EDTA, 0.5 mM EGTA, 0.1% Na-Deoxycholate, 0.5% N-lauroylsarcosine, 1× protease inhibitors). 12. After solubilization by sonication the pellet was chilled on ice and spun down at 5,000 g for 5 minutes at 4° C. 13. The supernatant—the RNP fraction (˜2 ml each) was collected. 14. 1.2 μl of 20% SDS; 0.8 μl 1 M DTT solution were added to 40 μl of RNP fractions and boiled for at least 1 min. 15. Samples were desalted on the pre-equilibrated G25 column (with 100 mM Tris-HCl, pH 7.4; 10 mM DTT; 0.6% SDS) and analyzed SDS-PAGE/Western Blotting.

### Western Blotting Analysis

Whole-cell lysates from approximately 0.2×10⁶ cells per each sample were separated on 12% SDS-PAGE gels and transferred to a nitrocellulose membrane. Immunoblots were stained overnight at 4° C. with the following primary antibodies: H2A.Z (1:2,000; Abcam ab4174), H2A.Z acetyl (K4+K7+K11) (1:2,000; Abcam ab18262) or TIP60 (1:1000; generous gift from Bruno Amati). All secondary horseradish peroxidase (HRP)-conjugated antibodies were diluted 1:5,000 and incubated for 1 hr at room temperature with TBST/5% BSA. Immuno-reactive proteins were detected using the Pierce® ECL system (Thermo Scientific #32106).

### Tandem Mass Spectrometry (LC-MS/MS)

Whole cell extracts were separated on a SDS-PAGE gels which were stained with Coomassie brilliant blue and the protein band of interest excised. Gel sections were reduced with 55 mM DTT, alkylated with 10 mM iodoacetamide (Sigma-Aldrich) and digested overnight with TPCK modified trypsin/LysC mix (Promega) at pH=8.3. Digestion was stopped with 1% TFA and peptides dried out in a SpeedVac to 10 μL. The peptide mixture was analyzed by positive ion mode LC-MS/MS using a high-resolution hybrid QExactive HF Orbitrap Mass Spectrometer (Thermo Fisher Scientific) via HCD with data-dependent analysis (DDA). Peptides were delivered and separated using an EASY-nLC nanoflow HPLC (Thermo Fisher Scientific) at 300 nL/min using self-packed 15 cm length×75 μm i.d. C18 fritted microcapillary columns. Solvent gradient conditions were: 90 min from 3% to 38% buffer B (100% acetonitrile) in buffer A: (0.9% acetonitrile/0.1% formic acid/99.0% water). The raw files were processed with MaxQuant version 1.5.2.8 with preset standard settings at a multiplicity of 1. Carbamidomethylation was set as a fixed modification while methionine oxidation and protein N-acetylation were considered as variable modifications. Search results were filtered with a false discovery rate of 0.01. Reverse hits and only by site identifications as well as potential contaminants were removed. MS data will be deposited to the ProteomeXChange Consortium via PRIDE upon acceptance of the manuscript.

Nuclear Chromatin (ChIP) and RNA Immunoprecipitation (nRIP)

ChIP was performed as follows. Cells were crosslinked with 1% formaldehyde for 10 min at r.t. Pellets of 1×10⁶ cells were used for immunoprecipitation and lysed for 10 minutes on ice and chromatin fragmented using a Branson 250 digital sonicator. Each ChIP was performed with 4 ug of antibody, incubated overnight at 4° C. A 50/50 slurry of protein A and protein G Dynabeads was used to capture enriched chromatin, which was then washed before reverse-crosslinking and proteinase K digestion at 65° C. AMPure XP beads were used to clean up and isolate ChIP DNA for subsequent library construction. The following antibodies were used for ChIP: H2A.Z (Abcam ab4174, lot GR3176820-1), acH2A.Z (Abcam ab18262, lot GR306397-1), TIP60 antibody; and IgG (Abcam ab171870). Fold enrichment was calculated using the formula 2^{(-ΔΔCt(ChIP/non-immune serum))}. Primer sets used for ChIP are listed in Table 2. nRIP was performed and crosslinked nuclei were collected as follows: 1. 60×10⁶ HL-60 cells were crosslinked with 1% formaldehyde (formaldehyde solution, freshly made: 50 mM HEPES-KOH; 100 mM NaCl; 1 mM EDTA; 0.5 mM EGTA; 11% formaldehyde) for 10 min at room temperature. Crosslinking was stopped by adding 1/10 volume of 2.66 M Glycine, kept for 5 min at room temperature and 10 minutes on ice. 3. Cell pellets were washed twice with ice-cold PBS (freshly supplemented with 1 mM PMSF). 4. Cell pellets were resuspended in cell lysis buffer (volume=4 mL): 1× Buffer (10 mM Tris pH 7.4; 10 mM NaCl; 0.5% NP-40, freshly supplemented with protease inhibitors (protease inhibitors cocktail: Roche Applied Science, Cat. No. 1836153, 1 tablet/375 μL H₂O; add as ×100), 0.1 mM PMSF, and 0.2 mM vanadyl complex (NEB). 5. Cells were incubated at 0° C. for 10-15 minutes and homogenized in a Dounce (10 strokes pestle A and 40 strokes pestle B). 6. Nuclei were recovered by centrifugation at 2,000 rpm for 10 minutes at 4° C. 7. Nuclei were resuspended in 3 ml of 1× Resuspension Buffer (50 mM HEPES-NaOH, pH 7.4; 10 mM MgCl₂) supplemented with 0.1 mM PMSF and 0.2 mM vanadyl complex. 8. DNaseI treatment (250 U/ml) was performed for 30 minutes at 37° C., and EDTA (final concentration 20 mM) added to stop the reaction. 9. Resuspended Nuclei were sonicated once for 20 s (1 pulse every 3 seconds) at 30% amplitude (Branson Digital Sonifer, Danbury, CT). Immunoprecipitation was performed as follows: 1. Before preclearing, the sample was adjusted to 1% Triton X-100; 0.1% sodium deoxycholate; 0.01% SDS; 140 mM NaCl; Protease inhibitors; 0.2 mM vanadyl complex; 0.1 mM PMSF. 2. Preclearing step: ˜50 μl magnetic beads (Protein A or G Magnetic Beads; #S1425S or #S1430S NEB) were added to the sample and incubation was carried out for 1 hr on a rocking platform at 4° C. 3. Beads were removed in the magnetic field. 4. The sample was then divided into five aliquots: (i) antibody of interest: (i) H2A.Z antibody (ab4174); (ii) acH2A.Z antibody (ab18262); (iii) TIP60 antibody; (iv) preimmune serum: IgG (ab171870); (v) no antibody, no serum (input). 5. 5 μg antibody or preimmune serum was added to the respective aliquot and incubation performed on a rocking platform overnight at 4° C. Input was stored at −20° C. after addition of SDS to 2% final concentration. Day II. 6. 200 μl of Protein A coated super-paramagnetic beads (enough to bind 8 μg IgG) were added to the samples and incubated on a rocking platform for 1 hr at 4° C. 7. Six washes of beads in the magnetic field were made with immunoprecipitation buffer (150 mM NaCl; 10 mM Tris-HCl, pH 7.4; 1 mM EDTA; 1 mM EGTA pH 8.0; 1% Triton X-100; 0.5% NP-40 freshly supplemented with 0.2 mM vanadyl complex and 0.2 mM PMSF) in a magnetic field. 8. Proteinase K treatment to release DNA/RNA into solution and to reverse the crosslinking was performed in 200 μl of: 100 mM Tris-HCl, pH 7.4; 0.5% SDS for the immunoprecipitated samples and in parallel for the input using 500 μg/ml of Proteinase K at 56° C. overnight. 9. Day III. Beads were removed in the magnetic field. 10. Phenol (pH 4.3) extraction was performed after addition of NaCl (0.2 M final concentration). 11. Ethanol precipitation (in the presence of glycogen); 3 hrs at −20° C. 12. The pellet was dissolved in 180 μl H₂O, heated at 75° C. for 3 minutes, and immediately chilled on ice. 13. Samples were treated with DNase I (250 U/ml) in the presence of RNase inhibitor at 300 U/ml in ×1 buffer #2 (NEB) at 37° C. for 30 minutes. 14. Phenol (pH 4.3) extraction and EtOH precipitation were repeated. 15. The RNA pellet was dissolved in 50 μl H₂O.

### RNA Electrophoretic Gel Mobility Shift Assays (RFMSAs)

RNA oligonucleotides (15 pmol) were end-labeled with [γ-³²P] ATP (Perkin Elmer) and T4 polynucleotide kinase (New England Biolabs). Reactions were incubated at 37° C. for 1 h and then passed through G-25 spin columns (GE Healthcare) according to the manufacturer's instructions to remove unincorporated radioactivity. Labeled samples were gel-purified on 10% polyacrylamide gels. Binding reactions were carried out in 10 μL volumes in the following buffer: 5 mM Tris pH 7.4, 5 mM MgCl₂, 1 mM DTT, 3% v/v glycerol, 100 mM NaCl. 5 μg of full length purified H2A.Z (Abcam) and TIP60 (SignalChem) proteins were incubated with 1.1 nM of ³²P-labeled ss RNAs. All reactions were assembled on ice and then incubated at room temperature for 30 min. Samples were loaded onto 6% native polyacrylamide gels (0.5×TBE) at 4° C. for 3 h at 140 V. Various concentrations (1 μM-10 mM) of H2A.Z and K7 acetylated H2A.Z peptides (AnaSpec) were incubated with a fixed amount of probe in 50 mM Tris-HCl pH 7.5, 100 mM NaCl, 0.4 mM EDTA, and 40 U/ml RNasin, 1 mM DTT, 50% glycerol in a final volume of 10 μL. Thereafter, 1 μl glutaraldehyde (0.2% final concentration) was added into the mixture and incubated at room temperature for 15 minutes. Samples were loaded onto 10% native polyacrylamide gels (0.5×TBE) at 4° C. for 3 h at 170 V. All gels were dried upon fixation with Methanol and Acetic Acid and exposed to X-ray film. RNA oligonucleotides are listed in Table 3.

### Quantification and Statistical Analysis

### ChIP-Sequencing Analyses

ChIP libraries' construction was performed and paired-end sequenced on NextSeq500 platform, at a reading length of 36 nucleotides. The resulting alignment files were trimmed to 150 bp and processed with trim_galore, Cutadapt and FastQC for adapter trimming and sequence quality control. ChIP-Seq reads were aligned to the hg38 human reference genome with STAR using “-outFilterMultimapNmax 1”, “-outFilterMatchNminOverLread 0.8”, “-alignIntronMax 1” “-alignEndsType EndToEnd”; the rest of the options were set to the default. AcH2AZ bam files were converted to bigWig using deepTools removing duplicated reads, normalizing by library size and transforming the values to “counts per million”. Duplicated reads were removed from H2AZ libraries using MarkDuplicates, and subsequently, analyzed using the DPOS algorithm from the DANPOS2 software. The resulting smoothed and quantile normalized wig files were converted into bigWig files using the wigToBigWig tool from UCSC. DeepTools was used to quantify and plot the heatmaps of the ChIP-Seq signal surrounding all the transcription start sites of genes annotated in the known Canonical table in UCSC. The meta-plots of the regions surrounding the transcription start sites were generated in R. The scatter plots were produced in R with the function smoothScatter using as input the arcsin transformed values of the area under the ChIP-Seq signal surrounding TSS extracted from the matrix used to generate the heatmaps. The scatter plots comparing the enrichment surrounding the TSS were generated by calculating the area under the signal in the bigWig using the ma, subsequently, the signal was transformed using. For acH2AZ ChIP-seq and IgG control mapped reads were processed with HOMER for assessing the statistical significance of ChIP-seq peaks. The peak size was set to 500 bp, and adjacent peaks within regions of 1000 bp were stitched together, and the ChIP signal vs IgG control peaks were filtered over regions of size 1000 bp, using a Poisson [symbol] -value threshold of 1·10⁻³, and a Poisson tag threshold of 32, with a peak fold change between ChIP signal vs IgG control of 4.

Comparison of the Enrichment of H2AZ and acH2AZ ChIP-Seq Signal Surrounding Gene TSS Loci (+/−2 kb)

The enrichment was calculated as the area under the curve produced by the ChIP-Seq signal around the TSS for every gene in the genome and subsequently transformed using the inverse sine function. The resulting values were used to compare the ChIP-Seq occupancy of DMSO samples against the DRB and ACTD samples. The comparison is shown as a scatterplot with a color gradient, where each TSS is a dot and regions of high concentration of dots are indicated in red while blue means no concentration.

### RIP-Sequencing Analyses

Immunoprecipitated RNA were processed for sequencing and RNAs were depleted of ribosomal RNA with Ribo-Zero™ Magnetic Gold Kit (cat. #MRZG126 Epicentre). Double stranded cDNA libraries were constructed using SCRIPTSEQ™ v2 RNA-Seq Library Preparation Kit (cat. #SSV21106 Epicentre). The libraries were subjected to final size-selection in 3% agarose gels. 250-500 bp fragments were excised and recovered using the Qiaquick Gel Extraction Kit (Qiagen). Paired-End reads generated by the sequencer (Illumina HiSeq-2000) were trimmed to 150 bp, and were further processed with trim_galore, Cutadapt and FastQC for adapter trimming and sequence quality control, and the pre-processed sequencing files were mapped to the GRCh38 human reference genome (release 88) using STAR with a 150 bp overhang length for the fragments used to construct the splice junction database. The RIP and IgG control mapped reads were processed with HOMER (PMID: 20513432) for assessing the statistical significance of RIP peaks. In this process, individual peaks spreading in bins of length 100 bp are stitched together into variable length regions, and the RIP signal vs IgG control peaks were filtered over regions of size 1000 bp, using a Poisson [symbol] -value threshold of 1·10⁻³, and a Poisson tag threshold of 32, with a peak fold change between RIP signal vs IgG control of 4. The statistically significant peaks were annotated based on their distance to the closer coding region using HOMER, which corresponded to 10403 gene loci overlapping with at least 1 significant RIPseq peak.

### RNA-Sequencing

RNA was extracted with TRI Reagent® (MRC). RNA samples were treated with DNase I (10 U of DNase I (Roche) per 3 microgram of total RNA; 37° C. for one hour; in the presence of RNase A inhibitor). RNAs were depleted of ribosomal RNA with Ribo-Zero™ Magnetic Gold Kit (cat. #MRZG126 Epicentre). Double stranded cDNA libraries were constructed using SCRIPTSEQ™ v2 RNA-Seq Library Preparation Kit (cat. #SSV21106 Epicentre). The libraries were subjected to final size-selection in 3% agarose gel. 250-500 bp fragments were excised and recovered using the Qiaquick Gel Extraction Kit (Qiagen). Libraries were sequenced on a Hi-Seq-2000 Illumina. The paired-end reads were pre-processed with trim_galore, Cutadapt and FastQC for adapter trimming and sequence quality control and aligned to the GRCh38 human reference genome (release 88) using STAR with a 150 bp overhang length for the fragments used to construct the splice junction database. The alignment files were analyzed with RSEM to estimate the expression levels of genes. To confirm the transcription of the significant RIP-Seq peaks, we overlapped the peak intervals with the RIP-Seq assemblies using the bedtools intersect Bed utility and the overlapping of SPEARs and late gene expression in the region of the transcription start site and coding regions were generated using NGSPLOT. All the statistical analyses and related plots were generated using ad-hoc R scripts (on the World Wide Web (www) at r-project.org).

### Motif Discovery

RNA binding motifs were identified according to the following steps:
1) Filtering on SPEARs expression
2) Prediction of 5′ and 3′ SPEARs boundaries from RNA-Seq
3) Search for a common motif in selected SPEARs
   In total, 13891 predicted promoter loci in HL60 (Broad ChromHMM) were subjected to coverage calculation. SPEARs were further selected according to their expression and the upper quartile subset (75^{th} percentile) was chosen for further analysis. Prior to Motif Discovery, 5′ and 3′ SPEARs boundaries were inferred from RNA-Seq. In particular, coverage tracks were scanned and 5′ and 3′ SPEARs boundaries were identified as a local drop in the level of coverage via the Friedman's SuperSmoother method (R, supsmu). In total, 1942 SPEARs were scanned for common motifs using the findGenomeMotif in RNA mode (Homer suite: on the world wide web at homer.ucsd.edu/homer/ngs) with option “-len 10,20,30” using Human promoters as a background (except those scanned for motifs). Motifs were filtered according to both significance (p<=e⁻¹⁰) and fold enrichment (observed vs expected >=200), identifying 3 enriched motifs (RM 3, 5 and 9, respectively).

The presence of discovered motifs in acH2A.Z and TIP60 RIP-Seq overlapping peaks was assessed by running findGenomeMotif with the -mknown option using not overlapping peaks as background. The same analysis was carried out for SINE, LINE and rRNA transcripts. Repetitive elements were retrieved from UCSC (RepeatMasker) and transcripts expressed >=2 fpkm were selected for motifs scanning. rRNA genomic region were retrieved from the UCSC Table Browser (Table: rmsk, repClass: rRNA).

### Statistical Analysis:

All the statistical analyses were performed using the R suite (on the World Wide Web (www) at r-project.org/). The statistical comparison of the distributions corresponding to ChIP-seq, RIP-seq and RNA-seq cumulative read count signals were performed using the Mann Whitney Wilcoxon signed rank test, with a confidence interval of ( [symbol] <0.05).

### Data Availability

Data are available on the gene omnibus database under the accession ID number: GSE 117663 (on the World Wide Web (www) at ncbi.nlm.nih.gov/geo/query/acc.cgi?acc=GSE117663

Enter token: azatswcwlzcndez

### Files in Database Submission:

SM3305784 ChIPseq HL-60 siMyc H2A.Z
GSM3305785 ChIPseq HL-60 siControl H2A.Z
GSM3305786 ChIPseq HL-60 siMyc acH2A.Z
GSM3305787 ChIPseq HL-60 siControl acH2A.Z
GSM3305788 ChIPseq HL-60 DMSO-treated H2A.Z
GSM3305789 ChIPseq HL-60 DRB-treated H2A.Z
GSM3305790 ChIPseq HL-60 ActD-treated H2A.Z
GSM3305791 ChIPseq HL-60 DMSO-treated acH2A.Z
GSM3305792 ChIPseq HL-60 DRB-treated acH2A.Z
GSM3305793 ChIPseq HL-60 ActD-treated acH2A.Z
GSM3305794 ChIPseq HL-60 DMSO-treated IgG
GSM3305795 ChIPseq HL-60 DRB-treated IgG
GSM3305796 ChIPseq HL-60 ActD-treated IgG
GSM3305797 ChIPseq HL-60 DMSO-treated input
GSM3305798 ChIPseq HL-60 DRB-treated input
GSM3305799 ChIPseq HL-60 ActD-treated input
GSM3305800 RIPseq HL-60 H2A.Z
GSM3305801 RIPseq HL-60a acH2A.Z
GSM3305802 RIPseq HL-60 Tip60
GSM3305803 RIPseq HL-60 IgG rabbit
GSM3305804 RIPseq HL-60 IgG sheep
GSM3305805 RIPseq HL-60 IgG goat
GSM3305806 RNAseq Hl-60
GSM3305807 nasRNAseq Hl-60
GSM3305808 nasRNAseq Hl-60 DMSO-treated
GSM3305809 nasRNAseq Hl-60 DRB-treated
GSM3305810 nasRNAseq Hl-60 ActD-treated
GSM3305784 ChIPseq HL-60 siMyc H2A.Z
GSM3305785 ChIPseq HL-60 siControl H2A.Z
GSM3305786 ChIPseq HL-60 siMyc acH2A.Z
GSM3305787 ChIPseq HL-60 siControl acH2A.Z
GSM3305788 ChIPseq HL-60 DMSO-treated H2A.Z
GSM3305789 ChIPseq HL-60 DRB-treated H2A.Z
GSM3305790 ChIPseq HL-60 ActD-treated H2A.Z
GSM3305791 ChIPseq HL-60 DMSO-treated acH2A.Z
GSM3305792 ChIPseq HL-60 DRB-treated acH2A.Z
GSM3305793 ChIPseq HL-60 ActD-treated acH2A.Z
GSM3305794 ChIPseq HL-60 DMSO-treated IgG
GSM3305795 ChIPseq HL-60 DRB-treated IgG
GSM3305796 ChIPseq HL-60 ActD-treated IgG
GSM3305797 ChIPseq HL-60 DMSO-treated input
GSM3305798 ChIPseq HL-60 DRB-treated input
GSM3305799 ChIPseq HL-60 ActD-treated input
GSM3305800 RIPseq HL-60 H2A.Z
GSM3305801 RIPseq HL-60a acH2A.Z
GSM3305802 RIPseq HL-60 Tip60
GSM3305803 RIPseq HL-60 IgG rabbit
GSM3305804 RIPseq HL-60 IgG sheep
GSM3305805 RIPseq HL-60 IgG goat
GSM3305806 RNAseq Hl-60
GSM3305807 nasRNAseq Hl-60
GSM3305808 nasRNAseq Hl-60 DMSO-treated
GSM3305809 nasRNAseq Hl-60 DRB-treated
GSM3305810 nasRNAseq Hl-60 ActD-treated

TABLE 1
short-hairpin RNAs Sequences

| c-MYC SPEARs shRNAs sequences | |
|---|---|
| (sense strand): | SEQ ID NO. |
| 5′-GCGGAGGGAAAGACGCUUU-3′ | 7 |
| 5′-CCAUCUUGAACAGCGUACA-3′ | 8 |
| 5′-CGGCAAAGGCCUGGAGGCA-3′ | 9 |
| 5′-GGGUAAUAACCCAUCUUGA-3′ | 10 |
| 5′-GCUGAAUUGUGCAGUGCAU-3′ | 11 |
| 5′-GGAUUUGGAAGCUACUAUA-3′ | 12 |
| 5′-GGAAACCUUGCACCUCGGA-3′ | 13 |
| 5′-GACAUCCAGGCGCGAUGAU-3′ | 14 |
| 5′-CCAUUACCGGUUCUCCAUA-3′ | 15 |
| 5′-GAAGCUACUAUAUUCACUU-3′ | 16 |
| 5′-GAAAGACGCUUUGCAGCAA-3′ | 17 |
| 5′-GGCCGUUUUAGGGUUUGUU-3′ | 18 |
| 5′-GGCACACUUACUUUACUUU-3′ | 19 |
| 5′-CGCUGAGCUGCAAACUCAA-3′ | 20 |
| 5′-CCAACCUGAAAGAAUAACA-3′ | 21 |
| 5′-GCGAUGAUCUCUGCUGCCA-3′ | 22 |
| 5′-GUAAUUUGCAAUCCUUAAA-3′ | 23 |
| 5′-GAGUAAUUUGCAAUCCUUA-3′ | 24 |
| 5′-GUCUAUGUACUUGUGAAUU-3′ | 25 |
| 5′-GCAAACUCAACGGGUAAUA-3′ | 26 |
| 5′-GCAAAAUCCAGCAUAGCGA-3′ | 27 |
| 5′-CAGUGCAUCGGAUUUGGAA-3′ | 28 |
| 5′-GCAAUCCUUAAAGCUGAAU-3′ | 29 |
| 5′-GGCUGGAAACUUGUUUUAA-3′ | 30 |
| 5′-CUAUGUACUUGUGAAUUAU-3′ | 31 |
| 5′-GCGUUUGCGGCAAAGGCCU-3′ | 32 |
| 5′-GAACAGCGUACAUGCUAUA-3′ | 33 |
| Negative Control siPool (siControl) | |
| Sequences (sense strand): | |
| 5′-UGUACGCGUCUCGCGAUUU-3′ | 34 |
| 5′-UAUACGCGGUACGAUCGUU-3′ | 35 |
| 5′-UUCGCGUAAUAGCGAUCGU-3′ | 36 |
| 5′-UCGGCGUAGUUUCGACGAU-3′ | 37 |
| 5′-UCGCGUAAGGUUCGCGUAU-3′ | 38 |
| 5′-UCGCGAUUUUAGCGCGUAU-3′ | 39 |
| 5′-UCGCGUAUAUACGCUACGU-3′ | 40 |
| 5′-UUUCGCGAACGCGCGUAAU-3′ | 41 |
| 5′-UCGUAUCGUAUCGUACCGU-3′ | 42 |
| 5′-UUAUCGCGCGUUAUCGCGU-3′ | 43 |
| 5′-UCUCGUAGGUACGCGAUCU-3′ | 44 |
| 5′-UCGUACUCGAUAGCGCAAU-3′ | 45 |
| 5′-UUUGCGAUACCGUAACGCU-3′ | 46 |
| 5′-UGCGUAAGGCAUGUCGUAU-3′ | 47 |
| 5′-UUAUCGGCAGUUCGCCGUU-3′ | 48 |
| 5′-UAGCGCGACAUCUAUCGCU-3′ | 49 |
| 5′-UCGUCGUAUCAGCGCGUUU-3′ | 50 |
| 5′-UACGCGAAACUGCGUUCGU-3′ | 51 |
| 5′-UCGACGAUAGCUAUCGCGU-3′ | 52 |
| 5′-UCGCGUAAUACGCGAUCGU-3′ | 53 |
| 5′-UCGCGAUAAUGUUACGCGU-3′ | 54 |
| 5′-UUAACGCGCUACGCGUAUU-3′ | 55 |
| 5′-UCGCGUAUAGGUAACGCGU-3′ | 56 |
| 5′-UUACGCGAUCACGUAACGU-3′ | 57 |
| 5′-UUAUCGCGCGUCGCGUAAU-3′ | 58 |
| 5′-UUACGUACUAGUGCGUACU-3′ | 59 |
| 5′-UAUACGCCGGUUGCGUAGU-3′ | 60 |
| 5′-UUCGCGUGCAUAGCGUAAU-3′ | 61 |
| 5′-UACGCGACCUAAUCGCGAU-3′ | 62 |
| 5′-UCGUACGCUGAACGCGUAU-3′ | 63 |

TABLE 2
Primer-Sequences used for Chromatin Immunoprecipitation qPCR for c-MYC and
PU.1 genes

| | | SEQ ID NO. |
|---|---|---|
| Forward mychip1 | 5′-GGC TAA TCC TCT ATG GGA GTC TGT C-3′ | 64 |
| Reverse mychip2 | 5′-TTT CTG AAT ACT AGT GAA AGT GCA-3′ | 65 |
| Forward mychip3 | 5′-TCA GAA AAA ATT GTG AGT CAG TGA-3′ | 66 |
| Reverse mychip4 | 5′-TTG TGG ACC GAG CCG GGG GAG TCA-3′ | 67 |
| Forward mychip5 | 5′-CCG GCT CGG TCC ACA AGC TCT CCA-3′ | 68 |
| Reverse mychip6 | 5′-TCT GCC TGT TCC AGA GCT GGG CTA-3′ | 69 |
| Forward mychip7 | 5′-ACA GGC AGA CAC ATC TCA GGG CTA-3′ | 2 |
| Reverse mychip8 | 5′-ATA GGG AGG AAT GAT AGA GGC ATA-3′ | 3 |
| Forward mychip9 | 5′-CTA CAC TAA CAT CCC ACG CTC TGA-3′ | 70 |
| Reverse mychip10 | 5′-AAC CGC ATC CTT GTC CTG TGA GTA-3′ | 1 |
| Forward mychip11 | 5′-AAG GAT GCG GTT TGT CAA ACA GTA-3′ | 71 |
| Reverse mychip12 | 5′-TCC TCA GCC GTC CAG ACC CTC GCA-3′ | 72 |
| Forward mychip13 | 5′-TAG AGT GCT CGG CTG CCC GGC TGA-3′ | 73 |
| Reverse mychip14 | 5′-TCT GAG AAG CCC TGC CCT TCT CGA-3′ | 74 |
| Forward mychip15 | 5′-GAA CGG AGG GAG GGA TCG CGC TGA-3′ | 75 |
| Reverse mychip16 | 5′-GTG CAA AGT GCC CGC CCG CTG CTA-3′ | 76 |
| Forward mychip17 | 5′-GAC TCT CCC GAC GCG GGG AGG CTA-3′ | 77 |
| Reverse mychip18 | 5′-CCC CAG TTA CCA TAA CTA CTC TGA-3′ | 78 |
| Forward mychip19 | 5′-GGA TCG GGG TAA AGT GAC TTG TCA-3′ | 79 |
| Reverse mychip20 | 5′-GCG GCT GCG GAG CGA TCT GGC TCA-3′ | 80 |
| Forward mychip21 | 5′-GCC AGA TCG CTC CGC AGC CGC TGA-3′ | 81 |
| Reverse mychip22 | 5′-ACA CCA CGT CCT AAC ACC TCT AGA-3′ | 82 |
| Forward PU.1ChIP | 5′-AAA GTC ATC CCT CTC AGT CCC AGC-3′ | 83 |
| Reverse PU.1ChIP | 5′-GAA GGG CCT GCC GCT GGG AGA TAG-3′ | 84 |

TABLE 3
RNA/DNA Oligonucleotides and peptides used for REMSA:

| | | SEQ ID NO. |
|---|---|---|
| RM9A | 5′-GGC GUG GCG GUG GGC GCG CAG U-3′ | 85 |
| Mut_RM9A | 5′-UUA UGU UAU UGU UUA UAU ACG U-3′ | 86 |
| Unrelated 1 (UR1) | 5′-GCG CCC UGC AGC CUG GUA CGC G-3′ | 87 |
| Unrelated 2 (UR2) | 5′-CUU UCC UCC ACU CUC CCU GGG A-3′ | 88 |
| Unrelated 3 (UR3) | 5′-GCC CUU UCC CCA GCC UUA GCG A-3′ | 89 |
| Mut1_UR2 | 5′-CUU UCA GAA CAG AGA CCU GGG A-3′ | 90 |
| Mut2_UR2 | 5′-CUU UCU CUU GUC UCU CCU GGG A-3′ | 91 |
| Mut_UR3 | 5′-GCC CUU UAA AAC UAA GGA GCG A-3′ | 92 |
| DM9F | 5′-GCC CTT TCC CCA GCC TTA GCG A-3′ | 93 |
| DM9R | 5′-TCG CTA AGG CTG GGG AAA GGG C-3′ | 94 |
| DM2F | 5′-CTT TCC TCC ACT CTC CCT GGG A-3′ | 95 |
| DM2R | 5′-TCC CAG GGA GAG TGG AGG AAA G-3′ | 96 |
| DM3F | 5′-GGC GTG GCG GTG GGC GCG CAG T-3′ | 97 |
| DM3R | 5′-ACT GCG CGC CCA CCG CCA CGC C-3′ | 98 |
| H2A.Z peptide | H-AGGKAGKDSGKAKTKAVSRS-OH | 99 |
| acH2A.Z peptide | H-AGGKAGK(Ac)DSGKAKTKAVSRS-OH | 100 |

All of the features disclosed herein may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A method for treating cancer in a subject in need thereof, comprising administering:
(i) an effective amount of an inhibitor of one or more S Phase Early RNAs (SPEARs) to the subject or
(ii) an effective amount of a cell derived from the subject, the cell having been contacted with an effective amount of an inhibitor of one or more SPEARs,
wherein the cancer is characterized by epigenetic dysregulation.

2. A method for preventing an onset or progression of cancer in a subject in need thereof, comprising administering:
(i) an effective amount of an inhibitor of one or more SPEARs to the subject or
(ii) an effective amount of a cell derived from the subject, the cell having been contacted with an effective amount of an inhibitor of one or more SPEARs,
wherein the subject is characterized by a pre-cancerous state comprising epigenetic dysregulation.

3. A method for treating a genetic disease or disorder associated with epigenetic dysregulation in a subject in need thereof, comprising administering:
(i) an effective amount of an inhibitor of one or more SPEARs to the subject or
(ii) an effective amount of a cell derived from the subject, the cell having been contacted with an effective amount of an inhibitor of one or more SPEARs.

4. A method for preventing an onset or progression of a genetic disease or disorder associated with epigenetic dysregulation in a subject in need thereof, comprising administering:
(i) an effective amount of an inhibitor of one or more SPEARs to the subject or
(ii) an effective amount of a cell derived from the subject, the cell having been contacted with an effective amount of an inhibitor of one or more SPEARs.

5. A method for resetting the formation of an active histone mark in a cancerous or pre-cancerous cell, comprising administering:
(i) an effective amount of one or more SPEARs or an inhibitor of one or more SPEARs to a subject or
(ii) contacting a cell derived from the subject with an effective amount of one or more SPEARs or an inhibitor of one or more SPEARs.

6. A method for restoring a replication origin complex associated with an undiseased state in a cell characterized by a genetic disease or disorder, comprising administering:
(i) an effective amount of one or more SPEARs or an inhibitor of one or more SPEARs to a subject or
(ii) contacting a cell derived from the subject with an effective amount of one or more SPEARs or an inhibitor of one or more SPEARs.

7. The method of any one of claims 1 to 6, wherein the inhibitor causes modulation of levels of expression of one or more genes controlled by the SPEAR.

8. The method of claim 7, wherein the modulation of levels of expression of one or more genes controlled by the SPEAR is upregulation of the genes.

9. The method of claim 7 or 8, wherein the modulation of levels of expression of one or more genes controlled by the SPEAR is downregulation of the genes.

10. The method of any one of claims 7 to 9, wherein the modulation of levels of expression of one or more genes controlled by the SPEAR is a restoration of levels of the one or more genes as compared to an untreated state.

11. The method of any one of claims 7 to 10, wherein the one or more genes is an oncogene or proto-oncogene.

12. The method of claim 10 or 11, wherein the gene is a myc gene.

13. The method of claim 12, wherein the myc gene is selected from c-myc (MYC), 1-myc (MYCL), and n-myc (MYCN).

14. The method of claim 11, wherein the gene is a tumor suppressor gene.

15. The method of any one of claims 6-14, wherein one or more SPEARs is overexpressed to generate an artificial replication origin complex.

16. The method of any one of claims 6-15, wherein one or more SPEARs is overexpressed to regulate the direction of replication.

17. The method of any one of claims 6-16, wherein one or more SPEARs is overexpressed and one or more SPEARs' inhibitors (i) slow the progression or prevent the deleterious direction of replication and activates the opposite direction of replication, and/or (ii) modulates the site of a trinucleotide repeat, optionally reducing the size of or reversing the expression of the trinucleotide repeat.

18. The method of any one of claims 6-17, wherein one or more SPEARs is overexpressed and one or more SPEARs' inhibitors treat or prevent a trinucleotide repeat disorder (“TRD”).

19. The method of claim 18, wherein the TRD is fragile X syndrome, fragile X-E syndrome, Huntington's disease (HD), spinocerebellar ataxias, a movement disorder, Dentatorubropallidoluysian atrophy, or autism.

20. The method of claim 18, wherein the TRD is a polyglutamine (PolyQ) disease and/or a non-polyglutamine disease.

21. The method of claim 20, wherein the polyglutamine disease is DRPLA (Dentatorubro-pallidoluysian atrophy), HD (Huntington's disease), SBMA (Spinobulbar muscular atrophy or Kennedy disease), SCA1 (Spinocerebellar ataxia Type 1), SCA2 (Spinocerebellar ataxia Type 2), SCA3 (Spinocerebellar ataxia Type 3 or Machado-Joseph disease), SCA6 (Spinocerebellar ataxia Type 6), SCA7 (Spinocerebellar ataxia Type 7), or SCA17 (Spinocerebellar ataxia Type 17).

22. The method of claim 20, wherein the non-polyglutamine disease is FXS (Fragile X syndrome), FXTAS (Fragile X-associated tremor ataxia syndrome), FRAXE (Fragile XE mental retardation), FRDA (Friedreich's ataxia), DM (Myotonic dystrophy), SCA8 (Spinocerebellar ataxia Type 8), SCA12 (Spinocerebellar ataxia Type 12) and premature ovarian failure (POF).

23. The method of any one of claims 18-22, wherein one or more SPEARs is overexpressed and one or more SPEARs' inhibitors treat or prevent a TRD by reversing the expansion of a trinucleotide repeat.

24. The method of claim 23, wherein the trinucleotide repeat is selected from CAG, CTG, CGG, and GAA.

25. The method of any one of claims 1 to 24, wherein the inhibitor reduces or substantially eliminates epigenetic mark activity associated with the SPEARs.

26. The method of any one of claims 1 to 25, wherein the inhibitor reduces or substantially eliminates formation and/or recycling of epigenetic marks.

27. The method of any one of claims 1 to 26, wherein the inhibitor reduces or substantially eliminates activation of genes.

28. The method of any one of claims 1 to 26, wherein the inhibitor causes the activation of genes.

29. The method of any one of claims 1 to 26, wherein the inhibitor reduces or substantially eliminates one or more of DNA methylation, histone modifications, and nucleosome remodeling.

30. The method of claim 29, wherein the histone modification is selected from one or more of histone acetylation, phosphorylation, methylation, ubiquitination, and proteolysis, and alterations in chromatin remodeling.

31. The method of claim 29 or 30, wherein the histone modification is histone acetylation.

32. The method of any one of claims 29-31, wherein the inhibitor causes modulation of disease-causing nucleotide expansions controlled by the SPEAR.

33. The method of any one of claims 1-32, wherein the inhibitor reduces or substantially eliminates interaction between the SPEAR and one or more histones or histone-associated proteins.

34. The method of claim 33, wherein the histone or histone-associated protein is one or more of H1, H2A, H2B, H3, and H4 protein, or a variant thereof.

35. The method of claim 33 or 34, wherein the histone or histone-associated protein is one or more of H2A.Z, or a variant thereof and H3.3, or a variant thereof.

36. The method of claim 33, wherein the histone or histone-associated protein is a histone acetyltransferase.

37. The method of claim 36, wherein the histone acetyltransferase is TIP60, or a variant thereof.

38. The method of any one of claims 1-37, wherein the inhibitor reduces or substantially eliminates interaction between the SPEAR and one or more components of ORC.

39. The method of claim 38, wherein the one or more components of ORC is selected from one or more of ORC1, ORC2, ORC3, ORC4, and ORC5, or a variant thereof.

40. The method of any one of claims 1-4 and 7-39, wherein the epigenetic dysregulation is dysregulation of one or more epigenetic marks.

41. The method of claim 40, wherein the epigenetic dysregulation of one or more epigenetic marks comprises the activation of additional epigenetic marks as compared to undiseased state and/or deactivation of epigenetic marks as compared to undiseased state.

42. The method of claim 40 or 41, wherein the epigenetic dysregulation is altered replication origin.

43. The method of claim 42, wherein the altered replication origin comprises the activation of additional replication origins as compared to undiseased state and/or deactivation of replication origins as compared to undiseased state.

44. The method of any one of claims 1 to 43, wherein the subject is afflicted with a cancer associated with epigenetic dysregulation.

45. The method of any one of claims 1, 2, 5 or 44, wherein the cancer is one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema, and Meigs' syndrome.

46. The method of any one of claims 1 to 45, wherein the SPEAR is a non-coding RNA.

47. The method of claim 46, wherein the SPEAR is a long noncoding RNA (lncRNA).

48. The method of claim 46 or 47, wherein the SPEAR is about 200 nucleotides or longer.

49. The method of any one of claims 46 to 48, wherein the SPEAR is encoded in a region adjacent to a promoter of an active gene.

50. The method of any one of claims 46 to 49, wherein the SPEAR is induced in early S phase of the cell cycle.

51. The method of any one of claims 46 to 50, wherein the SPEAR comprises one or more motifs selected from 3, 5, and 9.

52. The method of any one of claims 46 to 51, wherein the SPEAR comprises one or more RM9A motifs.

53. The method of any one of claims 46 to 52, wherein the SPEAR comprises one or more stem-loop-like structures.

54. The method of any one of claims 1 to 53, wherein the inhibitor is a small molecule.

55. The method of claim 54, wherein the small molecule directly or indirectly modulates interaction of the SPEAR with a histone or histone-associated protein or ORC.

56. The method of any one of claims 1 to 53, wherein the inhibitor is a nucleic acid.

57. The method of claim 56, wherein the nucleic acid is an RNA or DNA.

58. The method of claim 56 or 57, wherein the nucleic acid directly or indirectly modulates interaction of the SPEAR with a histone or histone-associated protein or ORC.

59. The method of any one of claims 56 to 58, wherein the nucleic acid comprising a sequence that is at least partially complementary to a portion of the SPEAR.

60. The method of any one of claims 56 to 59, wherein one or more nucleotides of the inhibitor are chemically modified.

61. The method of claim 60, wherein the chemical modification is selected from a locked nucleic acid (LNA), phosphorothioate, 2′-O-Methyl, 2′-O-Methoxyethyl, a 2′-O-alkyl-RNA unit, a 2′-OMe-RNA unit, a 2′-amino-DNA unit, a 2′-fluoro-DNA unit, a peptide nucleic acid (PNA) unit, a hexitol nucleic acids (HNA) unit, an INA unit, and a 2′-O-(2-Methoxyethyl)-RNA (2′ MOE RNA) unit.

62. The method of any one of claims 56 to 59, wherein the nucleic acid is an antisense oligonucleotide, or a small interfering RNA (siRNA).

63. The method of any one of claims 1 to 62, wherein the inhibitor modulates the expression and/or activity of the SPEAR.

64. The method of any one of claims 1 to 63, wherein the cell derived from the subject is derived from a biological sample.

65. The method of claim 64, wherein the biological sample comprises a biopsy, tissue or bodily fluid.

66. The method of claim 64 or 65, wherein the biological sample comprises one or more of tumor cells, cultured cells, stem cells, and differentiated cells.

67. The method of any one of claims 1 to 66, further comprising administering or contacting the cell with one or more epigenetic drugs.

68. The method of claim 67, wherein the epigenetic drug is a DNA methyltransferase inhibitor, optionally selected from azacytidine, ecitabine, zebularine, panobinostat, belinostat, dacinostat, quisinostat, tefinostat, acedinaline, entinostat, mocetinostat, chidamide, butyric acid, pivanex, phenylbutyric acid, and valproic acid.

69. The method of claim 67, wherein the epigenetic drug is a histone deacetylase inhibitor, optionally selected from vorinostat, romidepsin, trichostatin A and trapoxin A.

70. A method of making an epigenetic modulating agent, comprising:
(a) identifying an epigenetic modulating agent by:
(i) determining whether the agent binds to or interacts with one or more SPEARs;
(ii) classifying the agent as epigenetic modulating based on an ability to bind to or interact with one or more SPEARs; and
(b) formulating the agent for use in therapy, the therapy being selected from treatment or prevention of a cancer associated with epigenetic dysregulation or a genetic disease or disorder associated with epigenetic dysregulation.

71. The method of claim 70, wherein the agent reduces or substantially eliminates epigenetic mark activity associated with the SPEARs.

72. The method of any one of claim 70 or 71, wherein the agent reduces or substantially eliminates formation and/or recycling of epigenetic marks.

73. The method of any one of claims 70 to 72, wherein the agent reduces or substantially eliminates activation of genes.

74. The method of any one of claims 70 to 72, wherein the agent causes the activation of genes.

75. The method of any one of claims 70 to 73, wherein the agent reduces or substantially eliminates one or more of DNA methylation, histone modifications, and nucleosome remodeling.

76. The method of claim 75, wherein the histone modification is selected from one or more of histone acetylation, phosphorylation, methylation, ubiquitination, and proteolysis, and alterations in chromatin remodeling.

77. The method of claim 75 or 76, wherein the histone modification is histone acetylation.

78. The method of any one of claims 75-77, wherein the agent causes modulation of disease-causing nucleotide expansions controlled by the SPEAR.

79. The method of any one of claims 70-78, wherein the agent reduces or substantially eliminates interaction between the SPEAR and one or more histones or histone-associated proteins.

80. The method of claim 79, wherein the histone or histone-associated protein is one or more of H1, H2A, H2B, H3, and H4 protein, or a variant thereof.

81. The method of claim 79 or 80, wherein the histone or histone-associated protein is one or more of H2A.Z, or a variant thereof and H3.3, or a variant thereof.

82. The method of claim 79, wherein the histone or histone-associated protein is a histone acetyltransferase.

83. The method of claim 82, wherein the histone acetyltransferase is TIP60, or a variant thereof.

84. The method of any one of claims 70 to 83, wherein the epigenetic dysregulation is dysregulation of one or more epigenetic marks.

85. The method of claim 84, wherein the epigenetic dysregulation of one or more epigenetic marks comprises the activation of additional epigenetic marks as compared to undiseased state and/or deactivation of epigenetic marks as compared to undiseased state.

86. The method of claim 84 or 85, wherein the epigenetic dysregulation is altered replication origin.

87. The method of any one of claims 70 to 86, wherein the SPEAR is a non-coding RNA.

88. The method of claim 87, wherein the SPEAR is a long noncoding RNA (lncRNA).

89. The method of claim 87 or 88, wherein the SPEAR is about 200 nucleotides or longer.

90. The method of any one of claims 87 to 89, wherein the SPEAR is encoded in a region adjacent to a promoter of an active gene.

91. The method of any one of claims 87 to 90, wherein the SPEAR is induced in early S phase of the cell cycle.

92. The method of any one of claims 87 to 91, wherein the SPEAR comprises one or more motifs selected from 3, 5, and 9.

93. The method of any one of claims 87 to 92, wherein the SPEAR comprises one or more RM9A motifs.

94. The method of any one of claims 87 to 93, wherein the SPEAR comprises one or more stem-loop-like structures.

95. The method of any one of claims 70 to 94, wherein the agent comprises a small molecule.

96. The method of claim 95, wherein the small molecule directly or indirectly modulates interaction of the SPEAR with a histone or histone-associated protein or ORC.

97. The method of any one of claims 70 to 96, wherein the agent comprises a nucleic acid.

98. The method of claim 97, wherein the nucleic acid is an RNA or DNA.

99. The method of claim 97 or 98, wherein the nucleic acid directly or indirectly modulates interaction of the SPEAR with a histone or histone-associated protein or ORC.

100. The method of any one of claims 97 to 99, wherein the nucleic acid comprising a sequence that is at least partially complementary to a portion of the SPEAR.

101. The method of any one of claims 97 to 100, wherein one or more nucleotides of the agent is chemically modified.

102. The method of claim 101, wherein the chemical modification is selected from a locked nucleic acid (LNA), phosphorothioate, 2′-O-Methyl, 2′-O-Methoxyethyl, a 2′-O-alkyl-RNA unit, a 2′-OMe-RNA unit, a 2′-amino-DNA unit, a 2′-fluoro-DNA unit, a peptide nucleic acid (PNA) unit, a hexitol nucleic acids (HNA) unit, an INA unit, and a 2′-O-(2-Methoxyethyl)-RNA (2′ MOE RNA) unit.

103. The method of any one of claims 97 to 100, wherein the nucleic acid is an antisense oligonucleotide, or a small interfering RNA (siRNA).

104. The method of any one of claims 70 to 103, wherein the agent is capable of modulating the expression and/or activity of the SPEAR.

105. A method for evaluating a subject's response to an epigenetic modulating therapy, comprising evaluating a level of one or more of SPEARs in a biological sample from the subject, wherein:
(i) a reduced level of one or more of SPEARs compared to a pretreatment state is indicative of a response to therapy, and/or
(ii) an increased or substantially unchanged level of one or more of SPEARs compared to a pretreatment state is indicative of a lack of or poor response to therapy.

106. A method for predicting a subject's likelihood of response to an epigenetic modulating therapy, comprising evaluating a level of one or more of SPEARs in a biological sample from the subject, wherein:
(i) a high level of one or more of SPEARs is indicative of a high likelihood of response to the therapy, and/or
(ii) a low level of one or more of SPEARs is indicative of a low likelihood of response to the therapy.

107. The method of any one of claim 105 or 106, wherein the SPEAR is a non-coding RNA.

108. The method of claim 107, wherein the SPEAR is a long noncoding RNA (lncRNA).

109. The method of claim 107 or 108, wherein the SPEAR is about 200 nucleotides or longer.

110. The method of any one of claims 107 to 109, wherein the SPEAR is encoded in a region adjacent to a promoter of an active gene.

111. The method of any one of claims 107 to 110, wherein the SPEAR is induced in early S phase of the cell cycle.

112. The method of any one of claims 107 to 111, wherein the SPEAR comprises one or more motifs selected from 3, 5, and 9.

113. The method of any one of claims 107 to 112, wherein the SPEAR comprises one or more RM9A motifs.

114. The method of any one of claims 107 to 112, wherein the SPEAR comprises one or more stem-loop-like structures.

115. The method of any one of claims 105 to 114, wherein the biological sample comprises a biopsy, tissue or bodily fluid.

116. The method of claim 115, wherein the biological sample comprises one or more of tumor cells, cultured cells, stem cells, and differentiated cells.


SEQUENCE LISTING (104 sequences)

SEQ ID NO: 1 - DNA, 24 bp, Artificial Sequence
  Synthetic Sequence
aaccgcatccttgtcctgtgagta

SEQ ID NO: 2 - DNA, 24 bp, Artificial Sequence
  Synthetic Sequence
acaggcagacacatctcagggcta

SEQ ID NO: 3 - DNA, 24 bp, Artificial Sequence
  Synthetic Sequence
atagggaggaatgatagaggcata

SEQ ID NO: 4 - DNA, 20 bp, Artificial Sequence
  Synthetic Sequence
ggcttttgctctaacccaac

SEQ ID NO: 5 - DNA, 21 bp, Artificial Sequence
  Synthetic Sequence
actatgctgaagaccctacac

SEQ ID NO: 6 - DNA, 20 bp, Artificial Sequence
  Synthetic Sequence
gctctaacccaacaaatgcc

SEQ ID NO: 7 - RNA, 19 nt, Artificial Sequence
  Synthetic Sequence
gcggagggaaagacgcuuu

SEQ ID NO: 8 - RNA, 19 nt, Artificial Sequence
  Synthetic Sequence
ccaucuugaacagcguaca

SEQ ID NO: 9 - RNA, 19 nt, Artificial Sequence
  Synthetic Sequence
cggcaaaggccuggaggca

SEQ ID NO: 10 - RNA, 19 nt, Artificial Sequence
  Synthetic Sequence
ggguaauaacccaucuuga

SEQ ID NO: 11 - RNA, 19 nt, Artificial Sequence
  Synthetic Sequence
gcugaauugugcagugcau

SEQ ID NO: 12 - RNA, 19 nt, Artificial Sequence
  Synthetic Sequence
ggauuuggaagcuacuaua

SEQ ID NO: 13 - RNA, 19 nt, Artificial Sequence
  Synthetic Sequence
ggaaaccuugcaccucgga

SEQ ID NO: 14 - RNA, 19 nt, Artificial Sequence
  Synthetic Sequence
gacauccaggcgcgaugau

SEQ ID NO: 15 - RNA, 19 nt, Artificial Sequence
  Synthetic Sequence
ccauuaccgguucuccaua

SEQ ID NO: 16 - RNA, 19 nt, Artificial Sequence
  Synthetic Sequence
gaagcuacuauauucacuu

SEQ ID NO: 17 - RNA, 19 nt, Artificial Sequence
  Synthetic Sequence
gaaagacgcuuugcagcaa

SEQ ID NO: 18 - RNA, 19 nt, Artificial Sequence
  Synthetic Sequence
ggccguuuuaggguuuguu

SEQ ID NO: 19 - RNA, 19 nt, Artificial Sequence
  Synthetic Sequence
ggcacacuuacuuuacuuu

SEQ ID NO: 20 - RNA, 19 nt, Artificial Sequence
  Synthetic Sequence
cgcugagcugcaaacucaa

SEQ ID NO: 21 - RNA, 19 nt, Artificial Sequence
  Synthetic Sequence
ccaaccugaaagaauaaca

SEQ ID NO: 22 - RNA, 19 nt, Artificial Sequence
  Synthetic Sequence
gcgaugaucucugcugcca

SEQ ID NO: 23 - RNA, 19 nt, Artificial Sequence
  Synthetic Sequence
guaauuugcaauccuuaaa

SEQ ID NO: 24 - RNA, 19 nt, Artificial Sequence
  Synthetic Sequence
gaguaauuugcaauccuua

SEQ ID NO: 25 - RNA, 19 nt, Artificial Sequence
  Synthetic Sequence
gucuauguacuugugaauu

SEQ ID NO: 26 - RNA, 19 nt, Artificial Sequence
  Synthetic Sequence
gcaaacucaacggguaaua

SEQ ID NO: 27 - RNA, 19 nt, Artificial Sequence
  Synthetic Sequence
gcaaaauccagcauagcga

SEQ ID NO: 28 - RNA, 19 nt, Artificial Sequence
  Synthetic Sequence
cagugcaucggauuuggaa

SEQ ID NO: 29 - RNA, 19 nt, Artificial Sequence
  Synthetic Sequence
gcaauccuuaaagcugaau

SEQ ID NO: 30 - RNA, 19 nt, Artificial Sequence
  Synthetic Sequence
ggcuggaaacuuguuuuaa

SEQ ID NO: 31 - RNA, 19 nt, Artificial Sequence
  Synthetic Sequence
cuauguacuugugaauuau

SEQ ID NO: 32 - RNA, 19 nt, Artificial Sequence
  Synthetic Sequence
gcguuugcggcaaaggccu

SEQ ID NO: 33 - RNA, 19 nt, Artificial Sequence
  Synthetic Sequence
gaacagcguacaugcuaua

SEQ ID NO: 34 - RNA, 19 nt, Artificial Sequence
  Synthetic Sequence
uguacgcgucucgcgauuu

SEQ ID NO: 35 - RNA, 19 nt, Artificial Sequence
  Synthetic Sequence
uauacgcgguacgaucguu

SEQ ID NO: 36 - RNA, 19 nt, Artificial Sequence
  Synthetic Sequence
uucgcguaauagcgaucgu

SEQ ID NO: 37 - RNA, 19 nt, Artificial Sequence
  Synthetic Sequence
ucggcguaguuucgacgau

SEQ ID NO: 38 - RNA, 19 nt, Artificial Sequence
  Synthetic Sequence
ucgcguaagguucgcguau

SEQ ID NO: 39 - RNA, 19 nt, Artificial Sequence
  Synthetic Sequence
ucgcgauuuuagcgcguau

SEQ ID NO: 40 - RNA, 19 nt, Artificial Sequence
  Synthetic Sequence
ucgcguauauacgcuacgu

SEQ ID NO: 41 - RNA, 19 nt, Artificial Sequence
  Synthetic Sequence
uuucgcgaacgcgcguaau

SEQ ID NO: 42 - RNA, 19 nt, Artificial Sequence
  Synthetic Sequence
ucguaucguaucguaccgu

SEQ ID NO: 43 - RNA, 19 nt, Artificial Sequence
  Synthetic Sequence
uuaucgcgcguuaucgcgu

SEQ ID NO: 44 - RNA, 19 nt, Artificial Sequence
  Synthetic Sequence
ucucguagguacgcgaucu

SEQ ID NO: 45 - RNA, 19 nt, Artificial Sequence
  Synthetic Sequence
ucguacucgauagcgcaau

SEQ ID NO: 46 - RNA, 19 nt, Artificial Sequence
  Synthetic Sequence
uuugcgauaccguaacgcu

SEQ ID NO: 47 - RNA, 19 nt, Artificial Sequence
  Synthetic Sequence
ugcguaaggcaugucguau

SEQ ID NO: 48 - RNA, 19 nt, Artificial Sequence
  Synthetic Sequence
uuaucggcaguucgccguu

SEQ ID NO: 49 - RNA, 19 nt, Artificial Sequence
  Synthetic Sequence
uagcgcgacaucuaucgcu

SEQ ID NO: 50 - RNA, 19 nt, Artificial Sequence
  Synthetic Sequence
ucgucguaucagcgcguuu

SEQ ID NO: 51 - RNA, 19 nt, Artificial Sequence
  Synthetic Sequence
uacgcgaaacugcguucgu

SEQ ID NO: 52 - RNA, 19 nt, Artificial Sequence
  Synthetic Sequence
ucgacgauagcuaucgcgu

SEQ ID NO: 53 - RNA, 19 nt, Artificial Sequence
  Synthetic Sequence
ucgcguaauacgcgaucgu

SEQ ID NO: 54 - RNA, 19 nt, Artificial Sequence
  Synthetic Sequence
ucgcgauaauguuacgcgu

SEQ ID NO: 55 - RNA, 19 nt, Artificial Sequence
  Synthetic Sequence
uuaacgcgcuacgcguauu

SEQ ID NO: 56 - RNA, 19 nt, Artificial Sequence
  Synthetic Sequence
ucgcguauagguaacgcgu

SEQ ID NO: 57 - RNA, 19 nt, Artificial Sequence
  Synthetic Sequence
uuacgcgaucacguaacgu

SEQ ID NO: 58 - RNA, 19 nt, Artificial Sequence
  Synthetic Sequence
uuaucgcgcgucgcguaau

SEQ ID NO: 59 - RNA, 19 nt, Artificial Sequence
  Synthetic Sequence
uuacguacuagugcguacu

SEQ ID NO: 60 - RNA, 19 nt, Artificial Sequence
  Synthetic Sequence
uauacgccgguugcguagu

SEQ ID NO: 61 - RNA, 19 nt, Artificial Sequence
  Synthetic Sequence
uucgcgugcauagcguaau

SEQ ID NO: 62 - RNA, 19 nt, Artificial Sequence
  Synthetic Sequence
uacgcgaccuaaucgcgau

SEQ ID NO: 63 - RNA, 19 nt, Artificial Sequence
  Synthetic Sequence
ucguacgcugaacgcguau

SEQ ID NO: 64 - DNA, 25 bp, Artificial Sequence
  Synthetic Sequence
ggctaatcctctatgggagtctgtc

SEQ ID NO: 65 - DNA, 24 bp, Artificial Sequence
  Synthetic Sequence
tttctgaatactagtgaaagtgca

SEQ ID NO: 66 - DNA, 24 bp, Artificial Sequence
  Synthetic Sequence
tcagaaaaaattgtgagtcagtga

SEQ ID NO: 67 - DNA, 24 bp, Artificial Sequence
  Synthetic Sequence
ttgtggaccgagccgggggagtca

SEQ ID NO: 68 - DNA, 24 bp, Artificial Sequence
  Synthetic Sequence
ccggctcggtccacaagctctcca

SEQ ID NO: 69 - DNA, 24 bp, Artificial Sequence
  Synthetic Sequence
tctgcctgttccagagctgggcta

SEQ ID NO: 70 - DNA, 24 bp, Artificial Sequence
  Synthetic Sequence
ctacactaacatcccacgctctga

SEQ ID NO: 71 - DNA, 24 bp, Artificial Sequence
  Synthetic Sequence
aaggatgcggtttgtcaaacagta

SEQ ID NO: 72 - DNA, 24 bp, Artificial Sequence
  Synthetic Sequence
tcctcagccgtccagaccctcgca

SEQ ID NO: 73 - DNA, 24 bp, Artificial Sequence
  Synthetic Sequence
tagagtgctcggctgcccggctga

SEQ ID NO: 74 - DNA, 24 bp, Artificial Sequence
  Synthetic Sequence
tctgagaagccctgcccttctcga

SEQ ID NO: 75 - DNA, 24 bp, Artificial Sequence
  Synthetic Sequence
gaacggagggagggatcgcgctga

SEQ ID NO: 76 - DNA, 24 bp, Artificial Sequence
  Synthetic Sequence
gtgcaaagtgcccgcccgctgcta

SEQ ID NO: 77 - DNA, 24 bp, Artificial Sequence
  Synthetic Sequence
gactctcccgacgcggggaggcta

SEQ ID NO: 78 - DNA, 24 bp, Artificial Sequence
  Synthetic Sequence
ccccagttaccataactactctga

SEQ ID NO: 79 - DNA, 24 bp, Artificial Sequence
  Synthetic Sequence
ggatcggggtaaagtgacttgtca

SEQ ID NO: 80 - DNA, 24 bp, Artificial Sequence
  Synthetic Sequence
gcggctgcggagcgatctggctca

SEQ ID NO: 81 - DNA, 24 bp, Artificial Sequence
  Synthetic Sequence
gccagatcgctccgcagccgctga

SEQ ID NO: 82 - DNA, 24 bp, Artificial Sequence
  Synthetic Sequence
acaccacgtcctaacacctctaga

SEQ ID NO: 83 - DNA, 24 bp, Artificial Sequence
  Synthetic Sequence
aaagtcatccctctcagtcccagc

SEQ ID NO: 84 - DNA, 24 bp, Artificial Sequence
  Synthetic Sequence
gaagggcctgccgctgggagatag

SEQ ID NO: 85 - RNA, 22 nt, Artificial Sequence
  Synthetic Sequence
ggcguggcggugggcgcgcagu

SEQ ID NO: 86 - RNA, 22 nt, Artificial Sequence
  Synthetic Sequence
uuauguuauuguuuauauacgu

SEQ ID NO: 87 - RNA, 22 nt, Artificial Sequence
  Synthetic Sequence
gcgcccugcagccugguacgcg

SEQ ID NO: 88 - RNA, 22 nt, Artificial Sequence
  Synthetic Sequence
cuuuccuccacucucccuggga

SEQ ID NO: 89 - RNA, 22 nt, Artificial Sequence
  Synthetic Sequence
gcccuuuccccagccuuagcga

SEQ ID NO: 90 - RNA, 22 nt, Artificial Sequence
  Synthetic Sequence
cuuucagaacagagaccuggga

SEQ ID NO: 91 - RNA, 22 nt, Artificial Sequence
  Synthetic Sequence
cuuucucuugucucuccuggga

SEQ ID NO: 92 - RNA, 22 nt, Artificial Sequence
  Synthetic Sequence
gcccuuuaaaacuaaggagcga

SEQ ID NO: 93 - DNA, 22 bp, Artificial Sequence
  Synthetic Sequence
gccctttccccagccttagcga

SEQ ID NO: 94 - DNA, 22 bp, Artificial Sequence
  Synthetic Sequence
tcgctaaggctggggaaagggc

SEQ ID NO: 95 - DNA, 22 bp, Artificial Sequence
  Synthetic Sequence
ctttcctccactctccctggga

SEQ ID NO: 96 - DNA, 22 bp, Artificial Sequence
  Synthetic Sequence
tcccagggagagtggaggaaag

SEQ ID NO: 97 - DNA, 22 bp, Artificial Sequence
  Synthetic Sequence
ggcgtggcggtgggcgcgcagt

SEQ ID NO: 98 - DNA, 22 bp, Artificial Sequence
  Synthetic Sequence
actgcgcgcccaccgccacgcc

SEQ ID NO: 99 - protein, 20 aa, Artificial Sequence
  Synthetic Sequence
AGGKAGKDSGKAKTKAVSRS

SEQ ID NO: 100 - protein, 20 aa, Artificial Sequence
  Synthetic Sequence
AGGKAGKDSGKAKTKAVSRS

SEQ ID NO: 101 - RNA, 109 nt, Artificial Sequence
  Synthetic Sequence
cuuuccuccacucucccugggacucuugaucaaagcgcggcccuuuccccagccuuagcgaggcgcccugcagccugguacgcgcguggcguggcggugggcgcgcagu

SEQ ID NO: 102 - DNA, 109 bp, Artificial Sequence
  Synthetic Sequence
gaaaggaggtgagagggaccctgagaactagtttcgcgccgggaaaggggtcggaatcgctccgcgggacgtcggaccatgcgcgcaccgcaccgccacccgcgcgtca

SEQ ID NO: 103 - DNA, 109 bp, Artificial Sequence
  Synthetic Sequence
ctttcctccactctccctgggactcttgatcaaagcgcggccctttccccagccttagcgaggcgccctgcagcctggtacgcgcgtggcgtggcggtgggcgcgcagt

SEQ ID NO: 104 - DNA, 2590 bp, Artificial Sequence
  Synthetic Sequence
ggctgaggtaggaggtttgctttgagcccggggggatcgaggctgcagtgagctttgattgtgccactgcactccagcctgggtgacagaaggagaccctgtctcaaaaataataagaataataattaataataataggccaaaccaaatacccatcaccttctgctgtgcctcccctttccccaataaatccagtgtcttgctttcaaattttgtggttaaaaaagatgatgagtttctaagacgtgggggctaaagcttgtttggccgttttagggtttgttggaatttttttttcgtctatgtacttgtgaattatttcacgtttgccattaccggttctccatagggtgatgttcattagcagtggtgataggttaattttcaccatctcttatgcggttgaatagtcacctctgaaccactttttcctccagtaactcctctttcttcggaccttctgcagccaacctgaaagaataacaaggaggtggctggaaacttgttttaaggaaccgcctgtccttcccccgctggaaaccttgcacctcggacgctcctgctcctgcccccacctgacccccgccctcgttgacatccaggcgcgatgatctctgctgccagtagagggcacacttactttactttcgcaaacctgaacgcgggtgctgcccagagagggggcggagggaaagacgctttgcagcaaaatccagcatagcgattggttgctccccgcgtttgcggcaaaggcctggaggcaggagtaatttgcaatccttaaagctgaattgtgcagtgcatcggatttggaagctactatattcacttaacacttgaacgctgagctgcaaactcaacgggtaataacccatcttgaacagcgtacatgctatacacgcacccctttcccccgaattgttttctcttttggaggtggtggagggagagaaaagtttacttaaaatgcctttgggtgagggaccaaggatgagaagaatgttttttgtttttcatgccgtggaataacacaaaataaaaaatcccgagggaatatacattatatattaaatatagatcatttcagggagcaaacaaatcatgtgtggggctgggcaactagctaagtcgaagcgtaaataaaatgtgaatacacgtttgcgggttacatacagtgcactttcactagtattcagaaaaaattgtgagtcagtgaactaggaaattaatgcctggaaggcagccaaattttaattagctcaagactcccccccccccaaaaaaaggcacggaagtaatactcctctcctcttctttgatcagaatcgatgcattttttgtgcatgaccgcatttccaataataaaaggggaaagaggacctggaaaggaattaaacgtccggtttgtccggggaggaaagagttaacggtttttttcacaagggtctctgctgactcccccggctcggtccacaagctctccacttgccccttttaggaagtccggtcccgcggttcgggtaccccctgcccctcccatattctcccgtctagcacctttgatttctcccaaacccggcagcccgagactgttgcaaaccggcgccacagggcgcaaaggggatttgtctcttctgaaacctggctgagaaattgggaactccgtgtgggaggcgtgggggtgggacggtggggtacagactggcagagagcaggcaacctccctctcgccctagcccagctctggaacaggcagacacatctcagggctaaacagacgcctcccgcacggggccccacggaagcctgagcaggcggggcaggaggggcggtatctgctgctttggcagcaaattgggggactcagtctgggtggaaggtatccaatccagatagctgtgcatacataatgcataatacatgactccccccaacaaatgcaatgggagtttattcataacgcgctctccaagtatacgtggcaatgcgttgctgggttattttaatcattctaggcatcgttttcctccttatgcctctatcattcctccctatctacactaacatcccacgctctgaacgcgcgcccattaatacccttctttcctccactctccctgggactcttgatcaaagcgcggccctttccccagccttagcgaggcgccctgcagcctggtacgcgcgtggcgtggcggtgggcgcgcagtgcgttctcggtgtggagggcagctgttccgcctgcgatgatttatactcacaggacaaggatgcggtttgtcaaacagtactgctacggaggagcagcagagaaagggagagggtttgagagggagcaaaagaaaatggtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgag